(19) United States
(12) Patent Application Publication
(10) Pub. No.: US 2005/0244816 A1
(43) Pub. Date: Nov. 3, 2005

(54) Immunoassays, haptens, immunogens and antibodies for anti-HIV therapeutics

(75) Inventor: Johnny Valdez, Fremont, CA (US)

Correspondence Address: MORGAN, LEWIS & BOCKIUS LLP (SF), 2 PALO ALTO SQUARE, PALO ALTO, CA, 94306, US

(73) Assignee: Ark Diagnostics, Inc., Fremont, CA (US)

(21) Appl. No.: 11/019,419

(22) Filed: Dec. 20, 2004

Related U.S. Application Data
(60) Provisional application No. 60/531,552, filed on Dec. 19, 2003.

(51) Int. Cl. C12Q 1/70; A61K 31/522

(52) U.S. Cl. 435/5; 514/220; 514/263.38

(57) ABSTRACT

This invention provides compounds, methods, immunoassays, and kits relating to active, metabolically sensitive (“met-sensitive”) moieties of anti-HIV therapeutics, such as HIV protease inhibitors (PI) and HIV non-nucleoside reverse transcriptase inhibitors (NNRTI).

### CROSS-REFERENCE TO RELATED APPLICATIONS

This application claims priority to U.S. Provisional Application Ser. No. 60/531,552, filed on Dec. 19, 2003, the disclosure of which is incorporated herein by reference in its entirety for all purposes.

### BACKGROUND OF THE INVENTION

Acquired Immune Deficiency Syndrome (AIDS), the disease associated with infection from human immunodeficiency virus (HIV), is a disease that is pandemic and leaves practically no country in the world unaffected. The Joint United Nations Program on HIV/AIDS, UNAIDS, estimates that by the end of 2003, more than 40 million people will be living with HIV/AIDS. Unless the HIV lifecycle is interrupted by treatment, the virus infection spreads throughout the body and results in the destruction of the body's immune system and, ultimately, death.

While there is no cure for HIV infection, the introduction of antiretroviral drug therapy has resulted in a drastic reduction in the HIV morbidity and mortality rates. These retroviral drugs fall into four categories: non-nucleoside reverse transcriptase inhibitors (NNRTIs), such as nevirapine and efavirenz, protease inhibitors (PIs), such as indinavir and ritonavir, nucleoside reverse transcription inhibitors (NRTIs), such as emtricitabine and zidovudine, and fusion inhibitors, such as enfuvirtide. Combinations of these classes of drugs are prescribed according to the guidelines of highly active antiretroviral therapy (HAART), which seeks to reduce resistance, adverse reactions, and pill burdens, while improving efficacy. In spite of remarkable success with these new therapeutic regimens, not all patients respond optimally to the HIV combination drug therapies. This is due to multiple factors, but one of the most important is interpatient drug variability.

Levels of antiretroviral drugs in the blood may vary considerably from patient to patient for many reasons (e.g. drug-drug interactions in the body, differences in regimen adherence, differences in metabolism, differences in absorption). There is compelling scientific evidence that the concentrations of these anti-HIV therapeutics in the blood must be held in the right ranges in order to maximize their antiretroviral effect. Both variations above and below these ranges can present serious health risks to the patient. When anti-HIV therapeutic levels are low, replication of the virus is increased, which can lead to destruction of the immune system in the patient as well as development of HIV strains which are resistant to therapeutic treatment. When anti-HIV therapeutic levels are high, deleterious side effects can occur, such as renal problems with indinavir (Dieleman J P, et al., AIDS 13(4):473-478 (1999)), gastrointestinal disturbances with ritonavir (Gatti G, et al., AIDS 13(15):2083-2089 (1999)), hepatotoxicity with nevirapine (Gonzalez de Requena D, et al., AIDS 16(2):290-291 (2002), and CNS problems with efavirenz (Marzolini C, et al., AIDS 15(9):1192-1194 (2001)). While developing a ‘magic bullet’ drug without side effects remains an ideal objective, a more realistic goal is to utilize existing antiretroviral therapeutics in a more effective way. By ensuring that each patient has the appropriate levels of the anti-HIV therapeutic in his or her blood, the goal of suppressing virus replication with a minimum of side effects would be achieved. Therapeutic drug monitoring (TDM) offers a strategy for achieving this goal and thus improving antiretroviral therapy.

TDM involves measuring the amount of a particular drug in a blood sample. By frequently sampling the blood of an HIV-infected patient over time, the unique characteristics of the patient's response to anti-HIV therapeutics can be discovered. From this information, a individualized dosage schedule can be constructed which will maintain adequate drug concentrations throughout the dosing interval and avoid the overdosing or underdosing that could result in deleterious side effects.

Since TDM requires frequent testing, assays with high specificity, small sample volume requirements, reasonable cost, and rapid turnaround time are required. Currently most reports on TDM for PIs and NNRTIs have used high performance liquid chromatography (HPLC) and liquid chromatography-tandem mass spectrometry (LC/MS/MS) methods which are slow, labor-intensive, and expensive. Radioimmunoassays (RIA), while more amenable to high-throughput screening than HPLC or LC/MS/MS, suffer from regulatory, safety and waste disposal issues relating to the radioactive isotope label used in the assay. A TDM format that balances high-throughput screening with safety and environmental concerns would be ideal.

One promising candidate that combines these factors is non-isotopic immunoassays, such as those described in U.S. Pat. No. 3,817,837 (1974), the disclosure of which is incorporated herein by reference. Recently there have been several reports of non-isotopic immunoassays for PIs comprising PIs with an additional linker attached (Akeb, F. et al., J. Immunol. Methods 263(1-2):1-9 (2002); U.S. Pat. Application Publication Nos: 2003/0124518 and 2003/0100088). These assays detect not only unmetabolized, active anti-HIV therapeutics, but also detect the metabolized, inactive versions as well. Non-isotopic immunoassays for other classes of anti-HIV therapeutics do not currently exist. Their development would represent a significant advance in the art. This and other problems have been solved by the current invention.

In addition, currently no methods are available to detect only the unmetabolized, active version of the anti-HIV therapeutic and not the metabolized, inactive version. Their development would represent a significant advance in the art. This and other problems have been solved by the current invention.

### BRIEF SUMMARY OF THE INVENTION

The present invention enables the determination of the presence or the concentration of an active anti-HIV therapeutic in a sample. A variety of haptens, hapten-reactive partner conjugates, receptors, methods, and kits are useful in this determination.

Thus, in a first aspect, the invention provides a method for determining, in a sample from a host, the presence or the concentration of an anti-HIV therapeutic which inhibits HIV propagation. The anti-HIV therapeutic is selected from the group consisting of a HIV protease inhibitor (PI) and a non-nucleoside HIV reverse transcriptase inhibitor (NNRTI). The anti-HIV therapeutic comprises a metabolically-sensitive (“met-sensitive”) moiety that is transformed by the host to yield an inactivated metabolic product. The method of this first aspect comprises combining, in a solution, the sample with a receptor specific for the met-sensitive moiety where the receptor does not bind to the inactivated metabolic product, thus yielding an receptor-anti-HIV therapeutic complex. Finally, the method comprises detecting the complex.

In an exemplary embodiment, the receptor is an antibody. In an exemplary embodiment, the receptor further comprises a non-isotopic signal-generating moiety. In another exemplary embodiment, the PI is a member selected from lopinavir, saquinavir, amprenavir, indinavir, nelfinavir, tipranavir, atazanavir, and ritonavir. In yet another exemplary embodiment, the NNRTI is a member selected from efavirenz, nevirapine, delavirdine, and loviride. In still another exemplary embodiment, the method is a homogeneous immunoassay. In some exemplary embodiments, the detecting further comprises mixing the solution containing the receptor-anti-HIV therapeutic complex with a hapten-reactive partner conjugate comprising the met-sensitive moiety and a non-isotopic signal generating moiety; measuring the amount of the receptor bound to the hapten-reactive partner conjugate by monitoring a signal generated by the non-isotopic signal generating moiety; and correlating the signal with the presence or the concentration of the anti-HIV therapeutic in the sample. In other exemplary embodiments, the non-isotopic signal generating moiety is a member selected from an enzyme, a fluorogenic compound, a chemiluminescent compound, and combinations thereof. In another exemplary embodiment, the enzyme is glucose-6-phosphate dehydrogenase. In another exemplary embodiment, the met-sensitive moiety is a member selected from:

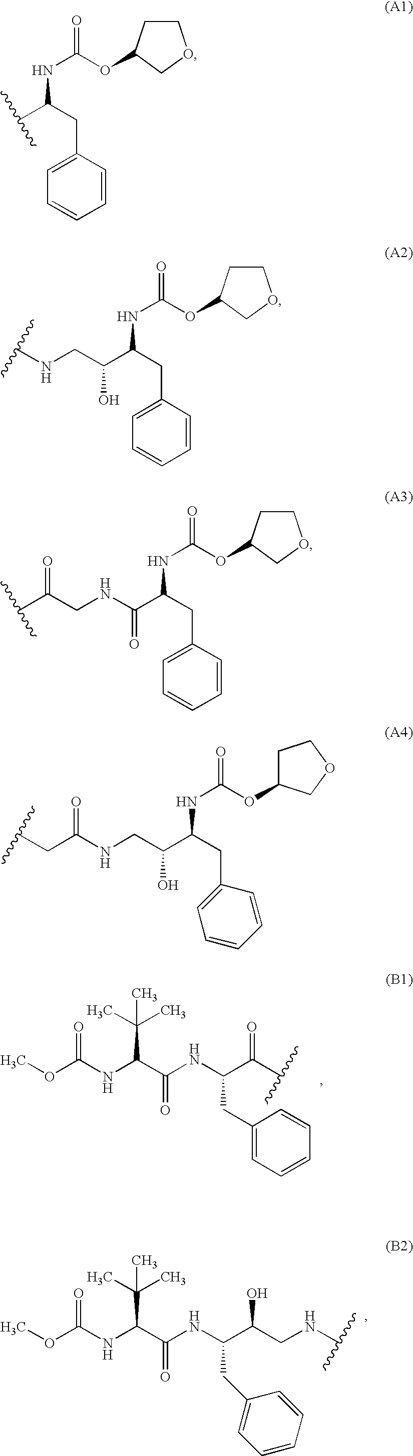

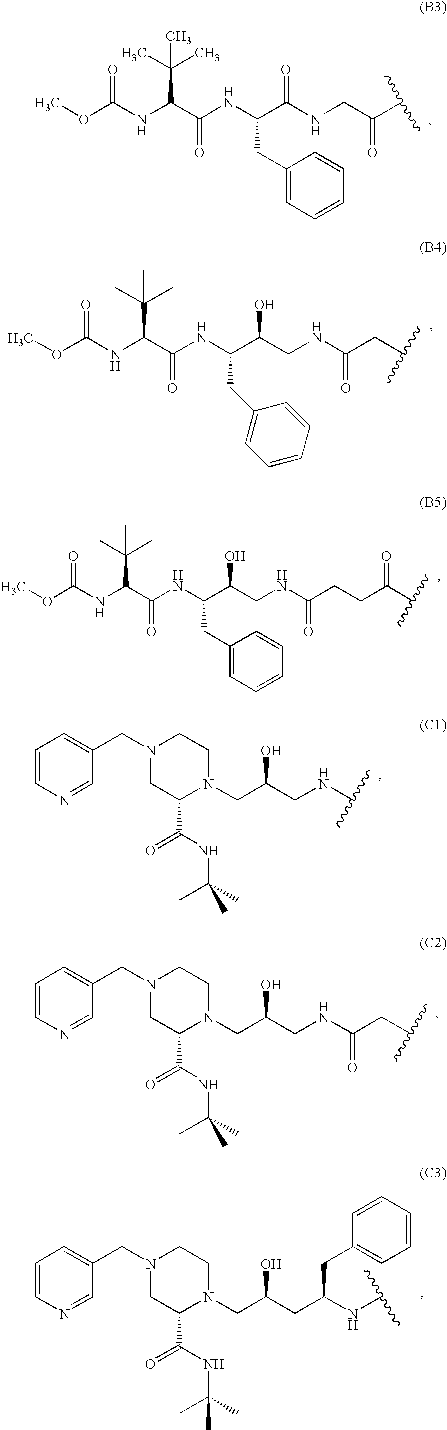

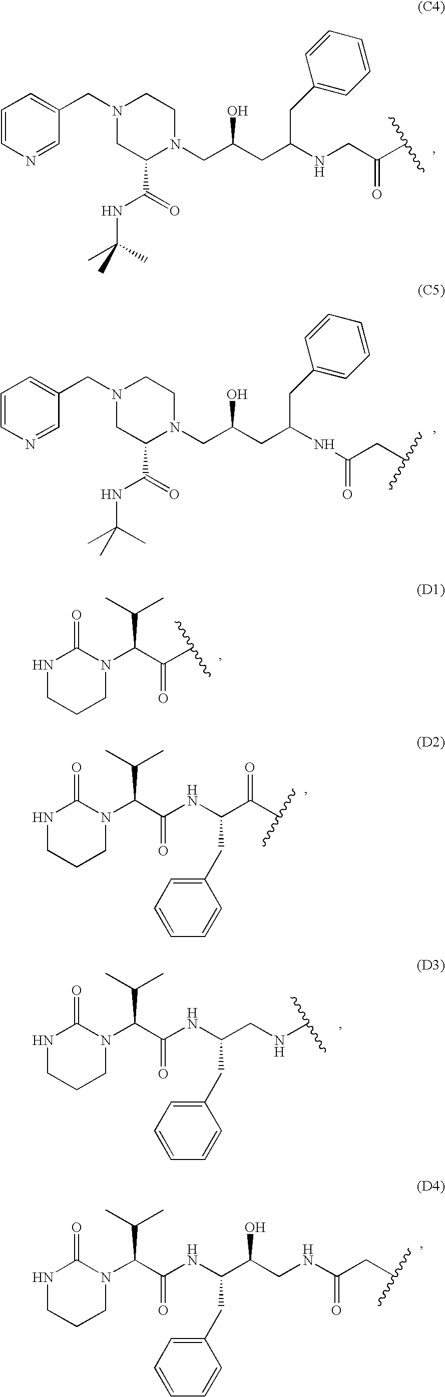

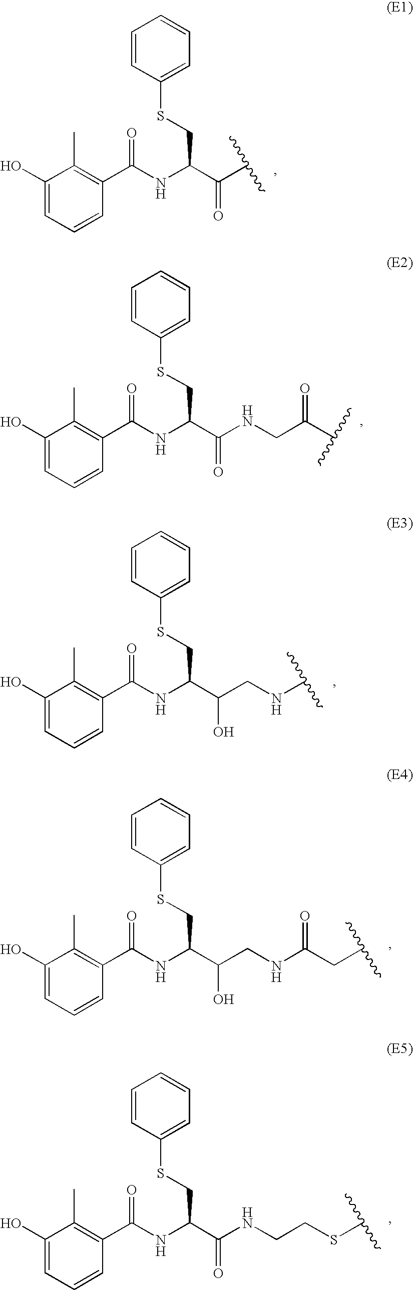

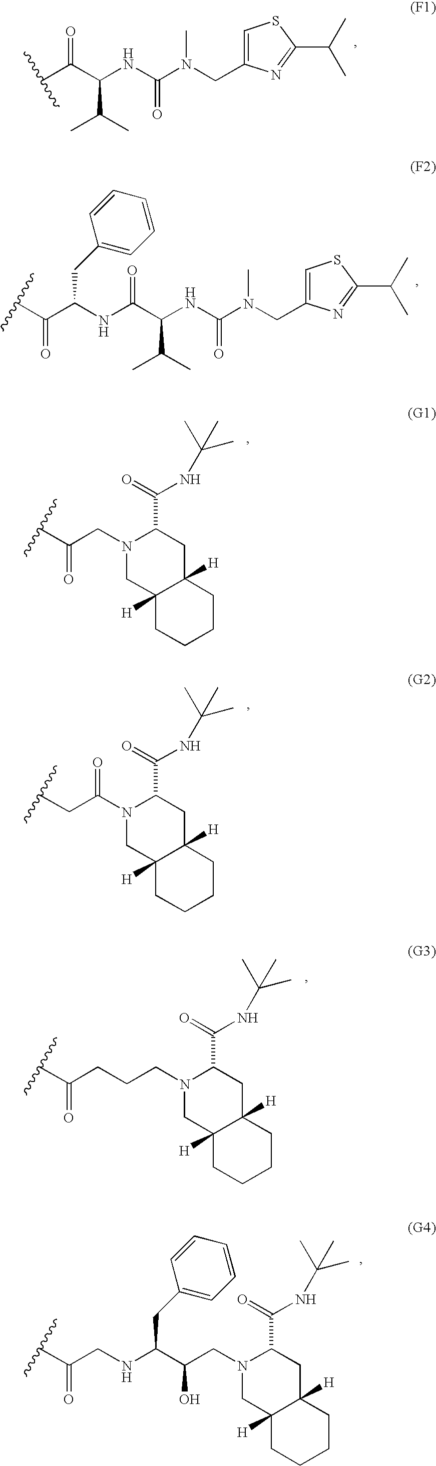

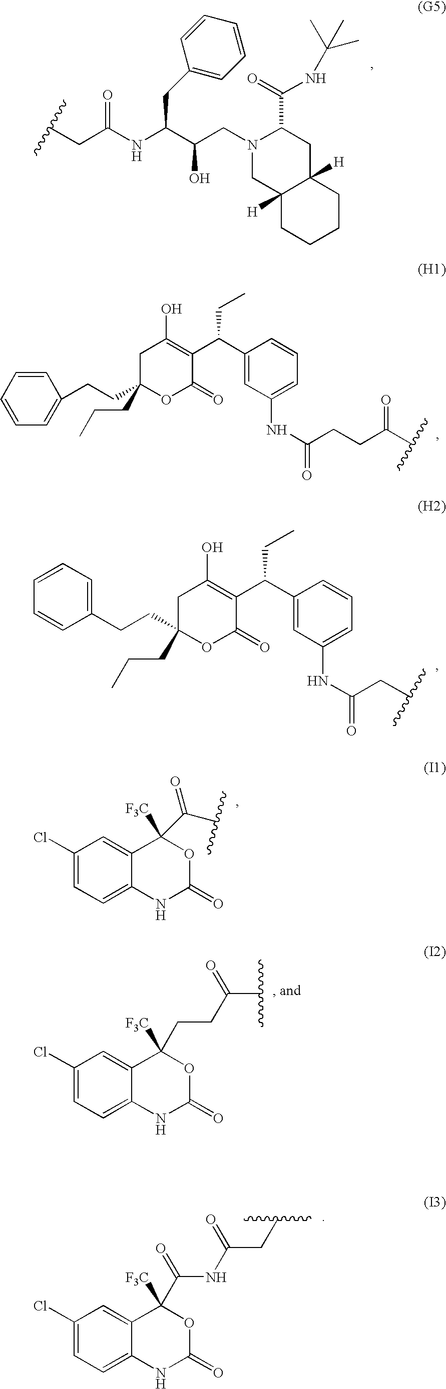

In a second aspect, the present invention provides a compound having the structure: I—(X)ₖ—(C═O)ₘ—(Y)ₙ-(L)ₚ-Q. In this structure, I is a met-sensitive moiety of an anti-HIV therapeutic, wherein the anti-HIV therapeutic is a member selected from PI and a NNRTI. X is a member selected from O, NH, S, and CH₂. Y is a member selected from O, NH, CH₂, OH, and CH₂—S. The symbols k, m, n, and p represent integers independently selected from 0 and 1. L is a linker consisting of from 1 to 40 carbon atoms arranged in a straight chain or a branched chain, saturated or unsaturated, optionally comprising carbonyl or carboxy moieties and containing up to two ring structures and 0-20 heteroatoms, with the provision that not more than two heteroatoms may be linked in sequence. Q, along with the atoms to which it is attached, forms a reactive functional moiety selected from the group consisting of amines, acids, esters, halogens, isocyanates, isothiocyanates, thiols, imidoesters, anhydrides, maleimides, thiolactones, diazonium groups and aldehydes. In another exemplary embodiment, PI is a member selected from amprenavir, atazanavir, indinavir, lopinavir, nelfinavir, ritonavir, saquinavir, and tipranavir. In another exemplary embodiment, NNRTI is a member selected from efavirenz, nevirapine, delavirdine, and loviride. In yet another exemplary embodiment, I is a member selected from (A1) to (I3) as described above. In still another exemplary embodiment, the symbol k represents 1, X is O, the symbol m represents 0, the symbol n represents 0, the symbol p represents 0, Q is succinimide, and I is a member selected from (A1) to (I3) as described above. In still another exemplary embodiment, the symbol k represents 1, X is O, the symbol m represents 0, the symbol n represents 0, the symbol p represents 0, Q is α haloacetyl, and I is a member selected from (A1) to (I3) as described above. In an exemplary embodiment, the invention provides a receptor that specifically binds to the compound having the structure: I—(X)ₖ—(C═O)ₘ—(Y)ₙ-(L)ₚ-Q. In an exemplary embodiment, the receptor is an antibody.

In a third aspect, the invention provides a compound having the structure: [1-(X)ₖ—(C═O)ₘ—(Y)ₙ-(L)ₚ-Z]ᵣ-P. In this structure, I, X, Y, L, k, m, n, and p are as described above. Z, along with the atoms to which it is attached, forms a moiety selected from the group consisting of —CONH—, —NHCO—, —NHCONH—, —NHCSNH—, —OCONH—, —NHOCO—, —S—, —NH(C═NH)—, —N═N—, and —NH—, —CH₂CO—, and maleimides. P is a member selected from an immunogenic carrier, a non-isotopic signal generating moiety, solid support, a polypeptide, polysaccharide, a synthetic polymer, and combinations thereof. The symbol r represents a number from 1 to the number of hapten binding sites in P. In an exemplary embodiment, PI is a member selected from amprenavir, atazanavir, indinavir, lopinavir, nelfinavir, ritonavir, saquinavir, and tipranavir. In another exemplary embodiment, NNRTI is a member selected from efavirenz, nevirapine, delavirdine, and loviride. In yet another exemplary embodiment, I is a member selected from (A1) to (I3) as described above. In an exemplary embodiment, the invention provides an receptor that specifically binds to the compound having the structure: [I—(X)ₖ—(C═O)ₘ—(Y)ₙ-(L)ₚ-Z]ᵣ-P.

In a fourth aspect, the invention provides an antigen for generating a receptor specific for a met-sensitive moiety of an anti-HIV therapeutic. In an exemplary embodiment, the receptor is an antibody. In another exemplary embodiment, the receptor specifically binds to a hapten comprising a met-sensitive moiety. In another exemplary embodiment, the receptor is selected from a Fab, Fab′, F(ab′)2, Fv fragment, and a single-chain antibody. In another exemplary embodiment, the receptor is specific for a met-sensitive moiety of amprenavir and has 10% or less cross-reactivity with atazanavir, indinavir, lopinavir, nelfinavir, ritonavir, saquinavir, and tipranavir. In another exemplary embodiment, the receptor is specific for a met-sensitive moiety of atazanavir and has 10% or less cross-reactivity with amprenavir, indinavir, lopinavir, nelfinavir, ritonavir, saquinavir, and tipranavir. In another exemplary embodiment, the receptor is specific for a met-sensitive moiety of indinavir and has 10% or less cross-reactivity with amprenavir, atazanavir, lopinavir, nelfinavir, ritonavir, saquinavir, and tipranavir. In another exemplary embodiment, the receptor is specific for a met-sensitive moiety of lopinavir and has 10% or less cross-reactivity with amprenavir, atazanavir, indinavir, nelfinavir, ritonavir, saquinavir, and tipranavir. In another exemplary embodiment, the receptor is specific for a met-sensitive moiety of nelfinavir and has 10% or less cross-reactivity with amprenavir, atazanavir, indinavir, lopinavir, ritonavir, saquinavir, and tipranavir. In another exemplary embodiment, the receptor is specific for a met-sensitive moiety of ritonavir and has 10% or less cross-reactivity with amprenavir, atazanavir, indinavir, lopinavir, nelfinavir, saquinavir, and tipranavir. In another exemplary embodiment, the receptor is specific for a met-sensitive moiety of saquinavir and has 10% or less cross-reactivity with amprenavir, atazanavir, indinavir, lopinavir, nelfinavir, ritonavir, and tipranavir. In another exemplary embodiment, the receptor is specific for a met-sensitive moiety of tipranavir and has 10% or less cross-reactivity with amprenavir, atazanavir, indinavir, lopinavir, nelfinavir, ritonavir and saquinavir. In another exemplary embodiment, the receptor is specific for a met-sensitive moiety of efavirenz and has 10% or less cross-reactivity with nevirapine, delavirdine, and loviride. In another exemplary embodiment, the receptor is specific for a met-sensitive moiety of nevirapine and has 10% or less cross-reactivity with efavirenz, delavirdine, and loviride. In another exemplary embodiment, the receptor is specific for a met-sensitive moiety of delavirdine and has 10% or less cross-reactivity with efavirenz, nevirapine, and loviride. In another exemplary embodiment, the receptor is specific for a met-sensitive moiety of loviride and has 10% or less cross-reactivity with efavirenz, nevirapine, and delavirdine. In another exemplary embodiment, the receptors have 5% or less cross-reactivity with the anti-HIV therapeutics that it was not specifically raised against. In another exemplary embodiment, the receptors have 3% or less cross-reactivity with the anti-HIV therapeutics that it was not specifically raised against. In another exemplary embodiment, the receptors have 1% or less cross-reactivity with the anti-HIV therapeutics that it was not specifically raised against. In another exemplary embodiment, I is a member selected from (A1) to (I3), and the receptor is a monoclonal antibody. In another exemplary embodiment, the invention is a receptor that substantially competes with the binding of the monoclonal antibody that specifically binds a met-sensitive moiety of the invention. This met-sensitive moiety which the receptor specifically binds can be part of a hapten or a hapten-reactive-partner conjugate. In another exemplary embodiment, the invention is a receptor that substantially competes with the binding of the monoclonal antibody that specifically binds a met-sensitive moiety of the invention. In some embodiments, the met-sensitive moiety is a member selected from (A1) to (I3). In another exemplary embodiment, the invention is a receptor that substantially competes with the binding of the monoclonal antibody that specifically binds a met-sensitive moiety of the invention. In another exemplary embodiment, the invention is a receptor that substantially competes with the binding of the receptor that specifically binds a met-sensitive moiety of the invention. In some embodiments, the receptor further comprises an antigen-binding domain.

In a fifth aspect, the invention provides a method of generating antibodies, comprising administering a compound to a mammal, the compound having the structure: [I—(X)ₖ—(C═O)ₘ—(Y)ₙ-(L)ₚ-Z]ᵣ—P. In this structure, I, X, Y, L, Z, P, k, m, n, p, and r are as described above. In an exemplary embodiment, PI is a member selected from amprenavir, atazanavir, indinavir, lopinavir, nelfinavir, ritonavir, saquinavir, and tipranavir. In another exemplary embodiment, NNRTI is a member selected from efavirenz, nevirapine, delavirdine, and loviride. In yet another exemplary embodiment, I is a member selected from (A1) to (I3) as described above.

In a sixth aspect, the invention provides a kit for determining, in a sample from a host, the presence or the concentration of an anti-HIV therapeutic which inhibits HIV propagation. The anti-HIV therapeutic is a member selected from a HIV protease inhibitor (PI) and a non-nucleoside HIV reverse transcriptase inhibitor (NNRTI) and the anti-HIV therapeutic comprises a met-sensitive moiety that is transformed by the host to yield an inactivated metabolic product. The kit comprises: (a) a receptor specific for the met-sensitive moiety where the receptor does not bind to the inactivated metabolic product, thus yielding a receptor-anti-HIV therapeutic complex; (b) a calibration standard; and (c) instructions on the use of the kit. In an exemplary embodiment, the kit further comprises (d) a hapten-reactive partner conjugate comprising the met-sensitive moiety and a non-isotopic signal generating moiety. In another exemplary embodiment, the non-isotopic signal generating moiety is a member selected from an enzyme, a fluorogenic compound, a chemiluminescent compound, and combinations thereof. In yet another exemplary embodiment, PI is a member selected from amprenavir, atazanavir, indinavir, lopinavir, nelfinavir, ritonavir, saquinavir, and tipranavir. In still another exemplary embodiment, NNRTI is a member selected from efavirenz, nevirapine, delavirdine, and loviride. In yet another exemplary embodiment, I is a member selected from (A1) to (I3) as described above. In other exemplary embodiments, the calibration standard comprises a matrix which is a member selected from human serum and buffered synthetic matrix.

The present invention also enables the determination of the presence or the concentration of NNRTIs, both active and inactive, in an sample through an “NNRTI Derivative” assay. Thus, in a seventh aspect, the invention provides a method for determining, in a sample from a host, the presence or the concentration of an NNRTI Derivative which inhibits HIV propagation. The method of this first aspect comprises combining, in a solution, the sample with a receptor specific for the NNRTI derivative, thereby generating a receptor-NNRTI complex. Finally, the method comprises detecting the complex.

In an exemplary embodiment, the receptor is an antibody. In an exemplary embodiment, the receptor further comprises a non-isotopic signal-generating moiety. In another exemplary embodiment, the NNRTI is a member selected from efavirenz, nevirapine, delavirdine, and loviride. In still another exemplary embodiment, the method is a homogeneous immunoassay. In some exemplary embodiments, the detecting further comprises mixing the solution containing the receptor-NNRTI complex with a hapten-reactive partner conjugate comprising the met-sensitive moiety and a non-isotopic signal generating moiety; measuring the amount of the receptor bound to the hapten-reactive partner conjugate by monitoring a signal generated by the non-isotopic signal generating moiety; and correlating the signal with the presence or the concentration of the receptor-NNRTI complex in the sample. In other exemplary embodiments, the non-isotopic signal generating moiety is a member selected from an enzyme, a fluorogenic compound, a chemiluminescent compound, and combinations thereof. In another exemplary embodiment, the enzyme is glucose-6-phosphate dehydrogenase. In another exemplary embodiment, the NNRTI Derivative is a member selected from (I4) to (J3).

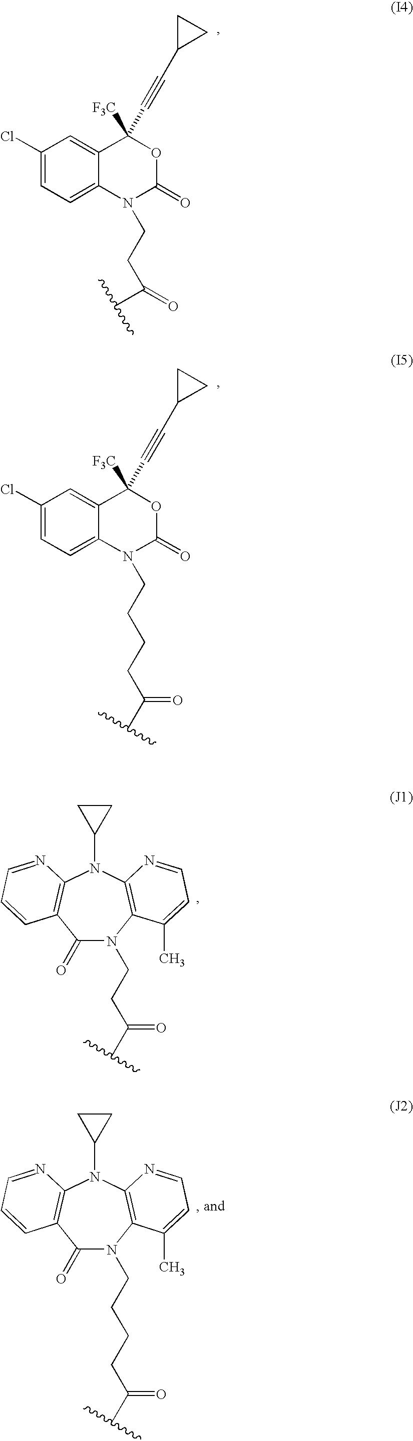

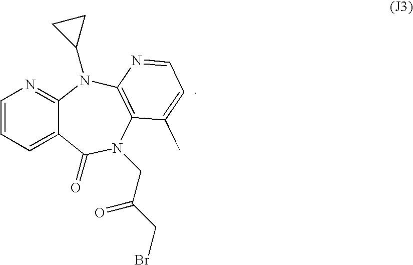

In an eighth aspect, the present invention provides a compound having the structure: I—(X)ₖ—(C═O)ₘ—(Y)ₙ-(L)ₚ-Q. In this structure, I is a NNRTI Derivative of an NNRTI. X is a member selected from O, NH, S, and CH₂. Y is a member selected from O, NH, CH₂, OH, and CH₂—S. The symbols k, m, n, and p represent integers independently selected from 0 and 1. L is a linker consisting of from 1 to 40 carbon atoms arranged in a straight chain or a branched chain, saturated or unsaturated, optionally comprising carbonyl or carboxy moieties and containing up to two ring structures and 0-20 heteroatoms, with the provision that not more than two heteroatoms may be linked in sequence. Q, along with the atoms to which it is attached, forms a reactive functional moiety selected from the group consisting of amines, acids, esters, halogens, isocyanates, isothiocyanates, thiols, imidoesters, anhydrides, maleimides, thiolactones, diazonium groups and aldehydes. In another exemplary embodiment, NNRTI is a member selected from efavirenz, nevirapine, delavirdine, and loviride. In yet another exemplary embodiment, I is a member selected from (I4) to (J3) as described above. In still another exemplary embodiment, the symbol k represents 1, X is O, the symbol m represents 0, the symbol n represents 0, the symbol p represents 0, Q is succinimide, and I is a member selected from (I4) to (J3) as described above. In still another exemplary embodiment, the symbol k represents 1, X is O, the symbol m represents 0, the symbol n represents 0, the symbol p represents 0, Q is a haloacetyl, and I is selected from (I4) to (J3) as described above. In an exemplary embodiment, the invention provides a receptor that specifically binds to the compound having the structure: I—(X)ₖ—(C═O)ₙ—(Y)ₙ-(L)ₚ-Q. In an exemplary embodiment, the receptor is an antibody.

In a ninth aspect, the invention provides a compound having the structure: [I—(X)ₖ—(C═O)ₘ—(Y)ₙ-(L)ₚ-Z]ᵣ-P. In this structure, I is a NNRTI Derivative of an NNRTI. X, Y, L, k, m, n, and p are as described above. Z, along with the atoms to which it is attached, forms a moiety selected from the group consisting of —CONH—, —NHCO—, —NHCONH—, —NHCSNH—, —OCONH—, —NHOCO—, —S—, —NH(C═NH)—, —N═N—, and —NH—, —CH₂CO—, and maleimides. P is a member selected from an immunogenic carrier, a non-isotopic signal generating moiety, solid support, a polypeptide, polysaccharide, a synthetic polymer, and combinations thereof. The symbol r represents a number from 1 to the number of hapten binding sites in P. In another exemplary embodiment, NNRTI is a member selected from efavirenz, nevirapine, delavirdine, and loviride. In yet another exemplary embodiment, I is a member selected from selected from (I4) to (J3) as described above. In an exemplary embodiment, the invention provides an receptor that specifically binds to the compound having the structure: [I—(X)ₖ—(C═O)ₘ—(Y)ₙ-(L)ₚ-Z]ᵣ-P.

In a tenth aspect, the invention provides an antigen for generating a receptor specific for a NNRTI Derivative of a NNRTI. In an exemplary embodiment, the receptor is an antibody. In another exemplary embodiment, the receptor specifically binds to a hapten comprising a NNRTI Derivative. In another exemplary embodiment, the receptor is selected from a Fab, Fab′, F(ab′)2, Fv fragment, and a single-chain antibody. In another exemplary embodiment, the receptor is specific for a NNRTI Derivative of efavirenz and has 10% or less cross-reactivity with nevirapine, delavirdine, and loviride. In another exemplary embodiment, the receptor is specific for a NNRTI Derivative of nevirapine and has 10% or less cross-reactivity with efavirenz, delavirdine, and loviride. In another exemplary embodiment, the receptor is specific for a NNRTI Derivative of delavirdine and has 10% or less cross-reactivity with efavirenz, nevirapine, and loviride. In another exemplary embodiment, the receptor is specific for a NNRTI Derivative of loviride and has 10% or less cross-reactivity with efavirenz, nevirapine, and delavirdine. In another exemplary embodiment, the receptors have 5% or less cross-reactivity with the anti-HIV therapeutics that it was not specifically raised against. In another exemplary embodiment, the receptors have 3% or less cross-reactivity with the anti-HIV therapeutics that it was not specifically raised against. In another exemplary embodiment, the receptors have 1% or less cross-reactivity with the anti-HIV therapeutics that it was not specifically raised against. In another exemplary embodiment, I is a member selected from (I4) to (J3), and the receptor is a monoclonal antibody. In another exemplary embodiment, the invention is a receptor that substantially competes with the binding of the monoclonal antibody that specifically binds a NNRTI Derivative of the invention. This NNRTI Derivative which the receptor specifically binds can be part of a hapten or a hapten-reactive-partner conjugate. In another exemplary embodiment, the invention is a receptor that substantially competes with the binding of the monoclonal antibody that specifically binds a NNRTI Derivative of the invention. In some embodiments, the NNRTI Derivative is a member selected from (I4) to (J3). In another exemplary embodiment, the invention is a receptor that substantially competes with the binding of the monoclonal antibody that specifically binds a NNRTI Derivative of the invention. In another exemplary embodiment, the invention is a receptor that substantially competes with the binding of the receptor that specifically binds a NNRTI Derivative of the invention. In some embodiments, the receptor further comprises an antigen-binding domain.

In a eleventh aspect, the invention provides a method of generating antibodies, comprising administering a compound to a mammal, the compound having the structure: [I—(X)ₖ—(C═O)ₘ—(Y)ₙ-(L)ₚ-Z]ᵣ—P. In this structure, I is a NNRTI Derivative of a NNRTI. X, Y, L, Z, P, k, m, n, p, and r are as described above. In another exemplary embodiment, NNRTI is a member selected from efavirenz, nevirapine, delavirdine, and loviride. In yet another exemplary embodiment, I is a member selected from (I4) to (J3) as described above.

In a twelvth aspect, the invention provides a kit for determining, in a sample from a host, the presence or the concentration of a NNRTI which inhibits HIV propagation. The kit comprises: (a) a receptor specific for the NNRTI Derivative. The kit can optionally comprise (b) a calibration standard; and (c) instructions on the use of the kit. In an exemplary embodiment, the kit optionally further comprises (d) a hapten-reactive partner conjugate comprising the NNRTI Derivative and a non-isotopic signal generating moiety. In another exemplary embodiment, the non-isotopic signal generating moiety is a member selected from an enzyme, a fluorogenic compound, a chemiluminescent compound, and combinations thereof. In still another exemplary embodiment, NNRTI is a member selected from efavirenz, nevirapine, delavirdine, and loviride. In yet another exemplary embodiment, I is a member selected from (I4) to (J3) as described above. In other exemplary embodiments, the calibration standard comprises a matrix which is a member selected from human serum and buffered synthetic matrix.

### BRIEF DESCRIPTION OF THE DRAWINGS

FIG. 1 is a calibration curve, alternatively known as a dose-response curve, for the anti-HIV therapeutic lopinavir. This graph is a representation of the change in optical density as a function of the concentration of lopinavir.

### DETAILED DESCRIPTION OF THE INVENTION

I. Introduction

The compounds, methods, and kits of the invention provide several new approaches to anti-HIV therapeutic drug monitoring. In a first new approach, the presence or the concentration of NNRTI in a sample can be ascertained through a non-isotopic immunoassay. This is accomplished through the attachment of a reactive functional group to an NNRTI, thus forming an “NNRTI Derivative”. This NNRTI Derivative can be utilized in TDM assays as is, or as further coupled to a reactive partner, in order to measure the amount of NNRTI, both active and inactive, in the sample.

The invention comprises compounds, methods, and kits which utilize NNRTI Derivatives. On one level, the invention comprises a hapten, which contains the NNRTI Derivative. The hapten can optionally further comprise a reactive functional group, linker, or a reactive functional group attached through a linker. The hapten can also optionally be attached to a reactive partner, such as a solid support, non-isotopic signal generating moiety, an immunogenic carrier, e.g., a carrier protein or enzyme, or combinations thereof. The hapten can be optionally linked to a reactive partner which comprises a signal-generating moiety in order to create an enzyme conjugate. Conjugation of the hapten with an immunogenic carrier can form a NNRTI Derivative Antigen, alternatively known as an immunogen. These immunogens can be used to raise antibodies against NNRTIs. The antibodies produced, or receptors based on these antibodies, can be incorporated into immunoassays, which determine the amount of the NNRTI in a subject. The materials described above can be incorporated into methods of determining the presence or the concentration of NNRTI in a sample, as well as methods of raising antibodies to these materials. Finally the materials described above can be incorporated into kits which can help assay anti-HIV therapeutic drug levels in patients.

In a second new approach, for the first time, differentiation is made between active and inactive forms of an anti-HIV therapeutic in a patient. Quantifying the active, or metabolically sensitive (“met-sensitive”), forms of PIs and NNRTIs provides several benefits to the individual and the community. First, monitoring of the active drug presence in a patient allows for the tailoring of a regimen that fits the patient's particular pharmacologic profile. This allows for more efficient dosing, better treatment, and the prolonged life of the subject. Second, more effective dosing leads to greater suppression of the virus, which in turn reduces the introduction of new HIV mutations in the community. This combination of more effective dosing and reduction in HIV mutations makes this invention a significant contribution to the art.

The invention comprises compounds, methods, and kits which utilize met-sensitive moieties of anti-HIV therapeutics. On one level, the invention comprises a hapten, which can contain the met-sensitive moiety. The hapten can optionally further comprise a reactive functional group, linker, or a reactive functional group attached through a linker. The hapten can also optionally be attached to a reactive partner, such as a solid support, non-isotopic signal generating moiety, an immunogenic carrier, e.g., a carrier protein or enzyme, or combinations thereof. The hapten can be optionally linked to a reactive partner which comprises a non-isotopic signal-generating moiety in order to create an enzyme conjugate. Conjugation of the hapten with an immunogenic carrier can form a met-sensitive antigen, alternatively known as an immunogen. These immunogens can be used to raise antibodies against the met-sensitive moieties of anti-HIV therapeutics. The antibodies produced can be incorporated into immunoassays, which determine the amount of the active anti-HIV therapeutic in a subject. The materials described above can be incorporated into methods of determining the concentration of anti-HIV therapeutics in a sample, as well as methods of raising antibodies to these materials. Finally the materials described above can be incorporated into kits which can help assay anti-HIV therapeutic drug levels in patients.

II. Definitions

Unless defined otherwise, all technical and scientific terms used herein have the same meaning as commonly understood by one of ordinary skill in the art to which this invention is related. The following terms are defined for purposes of the invention as described herein.

The symbol [symbol] , whether utilized as a bond or displayed perpendicular to a bond indicates the point at which the displayed moiety is attached to the remainder of the molecule, solid support, etc.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the invention may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al. (eds.), Vogel's Encyclopedia of Practical Organic Chemistry, 5th ed., Longman Scientific and Technical Ltd., Essex, 1991, pp. 809-816; and Heller, Acc. Chem. Res. 23: 128 (1990).

The compounds of the present invention may also contain unnatural proportions of atomic isotopes at one or more of the atoms that constitute such compounds. For example, the compounds may be radiolabeled with radioactive isotopes, such as, for example, tritium (³H), iodine-125 (¹²⁵I) or carbon-14 (¹⁴C). All isotopic variations of the compounds of the present invention, whether radioactive or not, are intended to be encompassed within the scope of the present invention.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “acyl” or “alkanoyl” by itself or in combination with another term, means, unless otherwise stated, a stable straight or branched chain, or cyclic hydrocarbon radical, or combinations thereof, consisting of the stated number of carbon atoms and an acyl radical on at least one terminus of the alkane radical. The “acyl radical” is the group derived from a carboxylic acid by removing the —OH moiety therefrom.

The term “alkyl,” by itself or as part of another substituent means, unless otherwise stated, a straight or branched chain, or cyclic hydrocarbon radical, or combination thereof, which may be fully saturated, mono- or polyunsaturated and can include divalent (“alkylene”) and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

Exemplary alkyl groups of use in the present invention contain between about one and about twenty five carbon atoms (e.g. methyl, ethyl and the like). Straight, branched or cyclic hydrocarbon chains having eight or fewer carbon atoms will also be referred to herein as “lower alkyl”. In addition, the term “alkyl” as used herein further includes one or more substitutions at one or more carbon atoms of the hydrocarbon chain fragment.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain, or cyclic carbon-containing radical, or combinations thereof, consisting of the stated number of carbon atoms and at least one heteroatom which is a member selected from the group consisting of O, N, Si, P and S, and wherein the nitrogen, phosphorous and sulfur atoms are optionally oxidized, and the nitrogen heteroatom is optionally be quaternized. The heteroatom(s) O, N, P, S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl” means, unless otherwise stated, a polyunsaturated, aromatic moiety that can be a single ring or multiple rings (preferably from 1 to 3 rings), which are fused together or linked covalently. The term “heteroaryl” refers to aryl groups (or rings) that contain from one to four heteroatoms which are members selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, tetrazolyl, benzo[b]furanyl, benzo[b]thienyl, 2,3-dihydrobenzo[1,4]dioxin-6-yl, benzo[1,3]dioxol-5-yl and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R″)═NR′″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R′″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present. In the schemes that follow, the symbol X represents “R” as described above.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” includes oxygen (O), nitrogen (N), sulfur (S), phosphorus (P) and silicon (Si).

The term “amino” or “amine group” refers to the group —NR′R″ (or N+RR′R″) where R, R′ and R″ are independently selected from the group consisting of hydrogen, alkyl, substituted alkyl, aryl, substituted aryl, aryl alkyl, substituted aryl alkyl, heteroaryl, and substituted heteroaryl. A substituted amine being an amine group wherein R′ or R″ is other than hydrogen. In a primary amino group, both R′ and R″ are hydrogen, whereas in a secondary amino group, either, but not both, R′ or R″ is hydrogen. In addition, the terms “amine” and “amino” can include protonated and quaternized versions of nitrogen, comprising the group —N⁺RR′R″ and its biologically compatible anionic counterions.

The term “aqueous solution” as used herein refers to a solution that is predominantly water and retains the solution characteristics of water. Where the aqueous solution contains solvents in addition to water, water is typically the predominant solvent.

“Antibody”, as used herein, refers to a protein functionally defined as a binding protein and structurally defined as comprising an amino acid sequence that is recognized by one of skill as being derived from the framework region of an immunoglobulin encoding gene of an animal producing antibodies. It includes whole antibody, functional fragments, modification or derivatives of the antibody. It can also be genetically manipulated product, or chimeric antibody.

“Antigen”, as used herein, refers to a compound that is capable of stimulating an immune response.

“Antibody-anti-HIV therapeutic complex”, as used herein, refers to the interaction of an antibody with an anti-HIV therapeutic. In an exemplary embodiment, the interaction is selected from hydrogen bonding, van der Waals interactions, repulsive electronic interactions, attractive electronic interactions, hydrophobic interactions, hydrophilic interactions and combinations thereof. In another exemplary embodiment, the interaction is covalent bonding or ionic bonding. Examples of antibody-anti-HIV therapeutic complexes include antigen-antibody, hapten-antibody, anti-HIV therapeutic fragment-antibody.

“Buffered synthetic matrix”, as used herein, refers to an aqueous solution comprising non-human constituents. Buffered synthetic matrices may include surface active additives, organic solvents, defoamers, buffers, surfactants, and anti-microbial agents. Surface active additives are introduced to maintain hydrophobic or low-solubility compounds in solution, and stabilize matrix components. Examples include bulking agents such as betalactoglobulin (BLG) or polyethyleneglycol (PEG); defoamers and surfactants such as Tween-20, Plurafac A38, Triton X-100, Pluronic 25R2, rabbit serum albumin (RSA), bovine serum albumin (BSA), and carbohydrates. Examples of organic solvents in buffered synthetic matrices include methanol and other alcohols. Various buffers may be used to maintain the pH of the synthetic matrix during storage. Illustrative buffers include HEPES, borate, phosphate, carbonate, tris, barbital and the like. Anti-microbial agents also extend the storage life of the matrix. An example of an anti-microbial agent used in this invention includes 2-methyl-4-isothiazolin-3-one hydrochloride.

“Immunogenic carrier”, as used herein, refers to any material which interacts with a hapten and stimulates an in vitro or in vivo immune response. Immunogenic carriers include proteins, glycoproteins, complex polysaccharides and nucleic acids that are recognized as foreign and thereby elicit an immunologic response from the host. Examples of carrier substances include keyhole limpet hemocyanin (KLH) and bovine serum albumin (BSA).

“Calibration standard”, as used herein, refers to an aqueous medium containing the anti-HIV therapeutic at a predetermined concentration. In an exemplary embodiment, a series of these calibration standards are available at a series of predetermined concentrations. In another exemplary embodiment, the calibration standard is stable at ambient temperature. In yet another exemplary embodiment, the calibration standards are in a synthetic matrix. In yet another exemplary embodiment, the calibration standards are in a non-synthetic matrix such as human serum.

“Concentration of an anti-HIV therapeutic”, as used herein, refers to the amount of anti-HIV therapeutic present in a sample. In an exemplary embodiment, the sample is synthetically produced, or taken from a mammal. The sample can be prepared in any convenient medium which does not interfere with the assay. In some exemplary embodiments, the sample is urine, blood, serum, breast milk, plasma, or saliva.

“Conjugate”, as used herein, refers to a molecule comprised of two or more moieties bound together, optionally through a linking group, to form a single structure. The binding can be made either by a direct connection (e.g. a chemical bond) between the subunits or by use of a linking group. Examples and methods of forming conjugates are further described in Hermanson, G. T., “Bioconjugate Techniques”, Academic Press: New York, 1996; and “Chemistry of Protein Conjugation and Cross-linking” by S. S. Wong, CRC Press, 1993, herein incorporated by reference.

“HIV protease inhibitor (PI)”, as used herein, refers to therapeutics that combats viral replication of HIV by blocking HIV's protease protein. This protein or enzyme is utilized by the virus to break up large viral proteins into smaller particles from which new HIV particles can be formed. PIs ensure that these new particles are immature and incapable of infecting new cells, thus inhibiting the HIV replication process.

“Homogeneous immunoassay”, as used herein, refers to an assay method where the complex is typically not separated from unreacted reaction components, but instead the presence of the complex is detected by a property which at least one of the reactants acquires or loses as a result of being incorporated into the complex. Homogeneous assays known in the art include systems involving fluorochrome and fluorochrome quenching pairs on different reagents (U.S. Pat. Nos. 3,996,345, 4,161,515, 4,256,834, and 4,264,968); enzyme and enzyme inhibitor pairs on different reagents (U.S. Pat. Nos. 4,208,479 and 4,233,401); chromophore and chromophore modifier pairs on different reagents (U.S. Pat. No. 4,208,479); and latex agglutination assays (U.S. Pat. Nos. 3,088,875, 3,551,555, 4,205,954, and 4,351,824).

“Human serum”, as used herein, refers to the aqueous portion of human blood remaining after the fibrin and suspended material (such as cells) have been removed.

“Inactivated metabolic product”, as used herein, refers to the transformation of chemical compounds within a living system which reduces or eliminates its therapeutic efficacy.

“Inhibits HIV propagation”, as used herein, refers to the viral load becoming significantly decreased or undetectable by the use of antiretroviral therapeutics, thus the risk of ultimate therapeutic failure is minimized. The presence of HIV RNA in plasma reflects viral replication, which in the presence of inadequate medications can lead to the development of resistant viral strains. If the viral load is suppressed to undetectable levels, the development of resistance is minimized, prolonging the durability of the antiretroviral response.

“Met-sensitive moiety”, as used herein, refers to a portion of an anti-HIV therapeutic to which an antibody binds. These met-sensitive portions are capable of binding specifically to corresponding antibodies, but do not themselves act as immunogens (or antigens) for preparation of the antibodies. Antibodies which recognize a met-sensitive portion can be prepared against compounds comprised of the defined portion linked to an immunogenic (or antigenic) carrier.

“Non-nucleoside HIV reverse transcriptase inhibitor (NNRTI)”, as used herein, refers to chemical compounds that prevent HIV replication by inhibiting the reverse transcriptase enzyme. This enzyme creates a deoxyribonucleic acid, or DNA, copy of HIV's genome from its ribonucleic acid, or RNA, template. Disrupting this RNA to DNA transcription event prevents HIV replication by disrupting the insertion of HIV's genome into an infected cell's genome.

“NNRTI Derivative”, as used herein, refers to chemical compounds which comprise an NNRTI molecule attached to one or more other moieties, such as linkers, reactive groups, etc. As a general rule, an NNRTI Derivative will not have a lower molecular weight than its respective NNRTI.

“Non-isotopic signal-generating moiety”, as used herein, refers to chemical compounds which do not use radioactive nuclei for detection purposes. By way of example, a non-isotopic signal-generating moiety is an enzyme, fluorescent compound, or a luminescent compound.

“Transformed”, as used herein, refers to the in vivo conversion of a chemical compound from an active form to an inactive form. In an exemplary embodiment, the chemical compound after transformation is less active or effective. In another exemplary embodiment, the molecular moiety that is transformed is metabolically sensitive.

The following abbreviations are used in the application: rt represents room temperature; ip represents interperitoneal; sc represents subcutaneous; FCA represents Freund's Complete Adjuvant; IFA represents Freund's Incomplete Adjuvant; HBSS represents Hank's Buffered Saline Solution; DMEM represents Dulbecco's Modified Eagle's Media; and HAT media is Hypoxanthine Aminopterin, Thymidine media.

III. Haptens Comprising Met-Sensitive Moieties or NNRTI Dertivatives

The essence of adaptive immunity is the ability of an organism to react to the presence of foreign substances and produce components (antibodies and cells) capable of specifically interacting with and protecting the host from their invasion. Not all foreign substances are capable of producing an immune response, however. Small molecules, although normally able to interact with the products of an immune response, often cannot cause a response on their own. These molecules are called haptens. Three examples of these haptens of use in this invention comprise met-sensitive moieties of PIs and NNRTIs, as well as NNRTI Derivatives. These compounds are alternatively known as haptens, haptens comprising met-sensitive moieties, or haptens comprising NNRTI Derivatives.

III. A. Hapten Examples

III. A. i) Haptens Comprising Met-Sensitive Moieties of PI

PIs are an important new class of drugs which have made a significant impact on the health care of AIDS patients since the first PI, saquinavir, was introduced to the marketplace in 1995. PIs combat viral replication of HIV by blocking HIV protease. This protease breaks up large viral proteins into smaller particles from which new HIV particles can be formed. PIs ensure that these new particles are immature and incapable of infecting new cells, thus inhibiting the HIV replication process. There are currently eight FDA approved protease inhibitors: amprenavir (Agenerase), atazanavir (Reyataz), fosamprenavir (Lexiva), indinavir (Crixivan), lopinavir/ritonavir (Kaletra), nelfinavir (Viracept), ritonavir (Norvir), saquinavir (Fortovase), and tipranavir.

The cytochrome P450 (CYP) enzyme 3A4 is central to the metabolism of many drugs, including PIs (Flexner C. et al. N Engl J Med 338:1281-1292 (1998)). The enzyme's activity serves to extensively metabolize and deactivate all currently known PIs, with the exception of nelfinavir, in hepatic microsomes as well as in the gastrointestinal tract. Therefore, it is important that antibodies used in an immunoassay be raised to that part of the molecule that undergoes metabolism in order to minimize cross-reactivity with deactivated metabolites. Consequently, the linkage both to the immunogenic carrier and the PI fragment must be on the opposite end of the molecule which undergoes biotransformation. Antibody cross-reactivity can be further minimized by designing haptens with a minimum of those moieties possessed by both the parent PI and its biotransformed metabolite derivative.

Descriptions of the met-sensitive moieties of PI are discussed below.

Amprenavir

Drug metabolism studies of amprenavir have been performed by several groups. One used human liver incubations and found that amprenavir metabolites arise from oxidative-reductive/oxidation ring opening (formation of diol and carboxylic acid metabolites) and oxidation of the tetrahydrofuran ring (formation of dihydrofuran metabolites. In addition, two monohydroxylated products were formed: one hydroxylation on the p-amino sulfonate aromatic ring and the other at the benzylic position (Singh, R, et al. Rapid Commun. Mass Spectrom. 10(9): 1019-1026 (1996)). Another group determined there to be two major amprenavir metabolites in humans. One metabolite resulted from dioxidation of the tetrahydrofuran ring and the second metabolite resulted from subsequent oxidation of the p-analine sufonate group (Sadler et al., J Clin Pharmacol. 41(4):386-396 (2001)).

Atazanavir

The major biotransformation pathways of atazanavir in humans consist of monooxygenation and dioxygenation. Other minor biotransformation pathways for atazanavir or its metabolites consisted of glucuronidation, N-dealkylation, hydrolysis, and oxygenation with dehydrogenation. Two minor metabolites of atazanavir in plasma have been characterized. Neither metabolite demonstrated in vitro antiviral activity. In vitro studies using human liver microsomes suggested that atazanavir is metabolized by CYP3A (information from Bristol-Myers Squibb Company atazanavir sulfate package insert; issued June 2003).

Indinavir

Disposition of [¹⁴C]indinavir was investigated in six healthy subjects after single oral administration of 400 mg (Balani et al., Drug Metabolism and Disposition 24 (12): 1389-1394 (1996)). The AUC value for the total radioactivity in plasma was 1.9 times higher than that of indinavir, indicating the presence of metabolites. The major excretory route was through feces, and the minor through urine. Mean recovery of radioactivity in the feces was 83.4%. In the urine, mean recoveries of the total radioactivity and unchanged indinavir were 18.7% and 11.0% of the dose, respectively. HPLC radioactivity and LC-MS/MS analyses of urine showed the presence of indinavir and low levels of quaternary pyridine N-glucuronide (M1), 2′,3′-transdihydroxy-indanylpyridin N-oxide (M2), 2′,3′-trans-dihydroxyindan (M3) and pyridine N-oxide (M4a) analogs, and despyridylmethyl analogs of M3 (M5) and indinavir (M6). M5 and M6 were the major metabolites in urine. The metabolic profile in plasma was similar to that in urine. Quantitatively, the metabolites in feces accounted for >47% of the dose which along with the urinary excretion of approximately 19%, suggested that the absorption of the drug was appreciable. In the feces, radioactivity was predominantly due to M3, M5, M6, and the parent compound. Thus, in urine and feces, the prominent metabolic pathways were oxidations and oxidative N-dealkylations. Excretion of the quaternary N-glucuronide metabolite in the urine is a minor metabolite in humans.

Lopinavir

The in vitro metabolism of lopinavir was determined in hepatic preparations from humans. It was shown that lopinavir was metabolized very extensively and rapidly by liver microsomes from humans (Kumar G N, et al. Drug Metab Dispos 1:86-91 (1999)). Twelve metabolites were chromatographically resolved and structurally identified. The predominate site of oxidative metabolism for lopinavir, yielding three major metabolites, is the cyclic urea moiety. Two of the metabolites are the epimeric C-4 hydroxy products of oxidation in the cyclic urea moiety and the other is the C-4 oxo product. The synthesis of the major metabolites was done to confirm the structures and to determine their antiviral activities (Sham et al. Bioorg Med Chem Lett. 11(11): ¹³⁵I-1353 (2001)). The NMR, mass spectroscopy and the HPLC retention times of the synthesized materials were identical to the isolated materials from metabolism studies. It was shown that the metabolism of lopinavir is essentially a deactivation reaction, because the major metabolites are significantly less potent inhibitors of the HIV protease than lopinavir.

Nelfinavir

Following the oral administration of nelfinavir mesylate to either healthy volunteers as a single dose or to HIV-infected patients as multiple doses, nelfinavir was the major circulating species in plasma, with several metabolites as minor components (Zheng K E, et al. Antimicrob Agents Chemother 45(4):1086-1093 (2001). Erratum in: Antimicrob Agents Chemother 45(8):2405 (2001)). The most abundant circulating metabolite involved the hydroxylation of nelfinavir on the t-butylamide group, and the less abundant metabolite presumably resulting from the 4′ hydroxylation on the benzamide moiety to form a catechol intermediate followed by methylation at the 3′ position. It was also demonstrated that the hydroxylation of nelfinavir on the t-butylamide group was not a deactivating reaction since it exhibited similar antiviral activity to nelfinavir in cell protection assays in vitro. In contrast, the 3′-methoxy-4′-hydroxynelfinavir metabolite showed EC50 fivefold higher than those of nelfinavir thus this biotransformation is a deactivating reaction.

Ritonavir

The metabolism and disposition of [¹⁴C]ritonavir, a potent, orally active HIV-1 PI, was investigated in HIV-negative male human volunteers (Denissen et al., Drug Metabolism and Disposition 25(4): 489-501 (1997)). Volunteers received a single 600 mg liquid oral dose. Ritonavir was cleared primarily via hepatobiliary elimination. Humans excreted 86.3% of the oral dose in feces and 11.3% in urine over 6 days. Radio-HPLC analysis of bile, feces, and urine indicated extensive metabolism of ritonavir to a number of oxidative metabolites involving metabolism at the terminal functional groups of the molecule. Plasma radioactivity consisted predominantly of unchanged parent drug. M2, the product of hydroxylation at the methine carbon of the terminal isopropyl moiety of ritonavir, was the only metabolite present in plasma and made up 30.4% of the total dose recovered in human excreta over 6 days. Plasma protein binding of ritonavir was high (99.3-99.5%) and was nonsaturable in humans at concentrations up to 30 μg/mL. Partitioning into the formed elements of whole blood was minimal.

Saquinavir

Research was performed to determine the potential of human hepatic and small-intestinal microsomes to metabolize saquinavir and to identify the enzyme systems involved in its biotransformation (Fitzsimmons M E, et al. Drug Metab Dispos. 25(2):256-266 (1997)). The results showed that saquinavir is oxidized by both human hepatic and small-intestine microsomes to multiple metabolites and that the CYP3A4 is the predominate enzyme involved in the biotransformation. The major metabolites of saquinavir were identified by LC/MS/MS [Liquid Chromatography Tandem Mass Spectrometry] as single hydroxylations on the octahydro-2-(1H)-isoquinolinyl and 1,1-dimethylethylamino groups, respectively.

Tipranavir

Tipranavir was shown to be metabolically stable. In preclinical pharmacokinetic studies and in in vitro rat, dog, and human primary hepatocyte incubations, tipranavir was stable (Koeplinger et al., Drug Metabolism and Disposition, 27 (9): 986-991 (1999)). Plasma metabolic profiles of tipranavir in rats or dogs showed only the parent drug. In vivo studies with tipranavir were consistent with the relative stability this compound exhibited in vitro.

III. A. ii) Haptens Comprising NNRTI Derivatives or Met-Sensitive Moieties of NNRTI

NNRTIs are another group of drugs used to treat HIV infection. These drugs stop HIV from multiplying by disrupting the function of HIV reverse transcriptase. This enzyme creates a deoxyribonucleic acid, or DNA, copy of HIV's genome from its ribonucleic acid, or RNA, template. Disrupting this RNA to DNA transcription event prevents HIV replication by disrupting the insertion of HIV's genome into an infected cell's genome. Examples of NNRTIs include efavirenz, nevirapine, loviride, and delavirdine.

The non nucleoside reverse transcriptase inhibitors (NNRTIs) are structurally and chemically dissimilar compounds that are highly potent inhibitors of HIV reverse transcriptase (RT). Unlike nucleoside analogs, the NNRTIs are not incorporated into the growing strand of HIV DNA, but directly inhibit the HIV RT by binding in a reversible and non competitive manner to the enzyme. The binding site is a hydrophobic pocket close to the polymerase catalytic site in the p66 subunit of RT, leading to a significant slowing rate of polymerization catalyzed by the enzyme. Because NNRTIs interact with a specific binding site on the enzyme, any slight variation brought about by a single point mutation can have a significant impact on the sensitivity of the virus to members of this group and high-level resistance can develop quickly (De Clercq E., et al. Medicinal Research Reviews 16: 125-157 (1996)). Other retroviral RT, such as hepatitis virus, herpes virus and mammalian enzyme systems are unaffected by these compounds.

NNRTIs are extensively metabolized in the liver through CYP, leading to pharmacokinetic interactions with compounds utilizing the same metabolic pathway, particularly PIs. PI concentrations in plasma are altered in the presence of NNRTIs (Smith P. F. et al., Clin. Pharmacokinet. 40(12):893-905 (2001); Aamoutse R E et al., Clin. Pharmacol. Ther., 71(1):57-67 (2002)).

Descriptions of the met-sensitive moieties of NNRTIs are discussed below.

Efavirenz

The metabolism profile of efavirenz was studied in humans using liquid chromatography/mass spectrometry (Mutlib A. E. et al., Drug Metab. Dispos. 27(11):1319-1333 (1999)). The metabolites were isolated, and structures were determined unequivocally by mass spectral and NMR analyses by comparing to synthetic standards. The major metabolite excreted in urine was O-glucuronide conjugate of the 8-hydroxylated metabolite. Efavirenz was also metabolized by direct conjugation with glucuronic acid, forming the N-glucuronide metabolite. Analyses of human plasma samples showed mostly unchanged efavirenz. Other metabolites present in plasma included O-glucuronide conjugate of the 8-hydroxylated metabolite, N-glucuronide metabolite, 8-OH efavirenz, 7-OH efavirnez, and the sulfate conjugate at the 7 carbon position.

Nevirapine

The pharmacokinetics and biotransformation of the antiretroviral agent nevirapine (NVP) after autoinduction were characterized in eight healthy male volunteers (Riska P et al., Drug Metab. Dispos. 27(8):895-901 (1999)). The pharmacokinetics and biotransformation of nevirapine was studied in subjects receiving 200 mg NVP tablets once daily for 2 weeks, followed by 200 mg twice daily for 2 weeks. Subsequently they received a single oral dose (solution) of 50 mg containing 100 μCi of [¹⁴C]NVP. Biological fluids were analyzed for total radioactivity, parent compound (HPLC/UV), and metabolites (electrospray liquid chromatography/mass spectroscopy and liquid chromatography/tandem mass spectroscopy). Mean recovery of radioactivity was 91.4%, with 81.3% excreted in urine and 10.1% recovered in the feces over a period of 10 days. Circulating radioactivity was evenly distributed between whole blood and plasma. At maximum plasma concentration, parent compound accounted for ˜75% of the circulating radioactivity. Mean plasma elimination half-lives for total radioactivity and NVP were 21.3 and 20.0 h, respectively. Several metabolites were identified in urine including 2-hydroxynevirapine glucuronide (18.6%), 3-hydroxynevirapine glucuronide (25.7%), 12-hydroxynevirapine glucuronide (23.7%), 8-hydroxynevirapine glucuronide (1.3%), 3-hydroxynevirapine (1.2%), 12-hydroxynevirapine (0.6%), and 4-carboxy-nevirapine (2.4%). Greater than 80% of the radioactivity in urine was made up of glucuronidated conjugates of hydroxylated metabolites of NVP. Thus, cytochrome P-450 metabolism, glucuronide conjugation, and urinary excretion of glucuronidated metabolites represent the primary route of NVP biotransformation and elimination in humans. Only a small fraction of the dose (2.7%) was excreted in urine as parent compound.

III. B. Methods of Making the Haptens

In addition to the met-sensitive or NNRTI Derivative moieties, the haptens of the invention can further comprise reactive functional groups, linkers, or both. Reactive functional groups and/or linkers can be used in order to create covalent linkages between the hapten and other compounds, such as reactive partners.

III. B. i) Reactive Functional Groups

Reactive functional groups can be represented by either Q, which represents a reactive functional group, or (-L-Q), which represents a reactive functional group Q that is attached to the met-sensitive moiety, NNRTI Derivative, or the reactive partner by a covalent linkage L. In an exemplary embodiment, Q, along with the atoms to which it is attached, forms a reactive functional group which is a member selected from amines, carboxylic acids, esters, halogens, isocyanates, isothiocyanates, thiols, imidoesters, anhydrides, maleimides, thiolactones, diazonium groups, aldehydes, acrylamide, an acyl azide, an acyl nitrile, an alkyl halide, an aniline, an aryl halide, an azide, an aziridine, a boronate, a carboxylic acid, a diazoalkane, a haloacetamide, a halotriazine, a hydrazine, a hydrazide, an imido ester, a phosphoramidite, a reactive platinum complex, a sulfonyl halide, and a photoactivatable group. In another exemplary embodiment, the point of attachment of the reactive group to the met-sensitive moiety is designated by “ [symbol] ”.

III. B. ii) Linkers

In some embodiments, the reactive functional group further comprises a linker, L. The linker is used to covalently attach a reactive functional group to the met-sensitive moiety or NNRTI Derivative of the invention. When present, the linker is a single covalent bond or a series of stable bonds. Thus, the reactive functional group may be directly attached (where the linker is a single bond) to the met-sensitive moiety or NNRTI Derivative or attached through a series of stable bonds. When the linker is a series of stable covalent bonds the linker typically incorporates 1-20 nonhydrogen atoms selected from the group consisting of C, N, O, S, and P. In addition, the covalent linkage can incorporate a platinum atom, such as described in U.S. Pat. No. 5,714,327. When the linker is not a single covalent bond, the linker may be any combination of stable chemical bonds, optionally including, single, double, triple or aromatic carbon-carbon bonds, as well as carbon-nitrogen bonds, nitrogen-nitrogen bonds, carbon-oxygen bonds, sulfur-sulfur bonds, carbon-sulfur bonds, phosphorus-oxygen bonds, phosphorus-nitrogen bonds, and nitrogen-platinum bonds. In an exemplary embodiment, the linker incorporates less than 15 nonhydrogen atoms and are composed of a combination of ether, thioether, thiourea, amine, ester, carboxamide, sulfonamide, hydrazide bonds and aromatic or heteroaromatic bonds. Typically the linker is a single covalent bond or a combination of single carbon-carbon bonds and carboxamide, sulfonamide or thioether bonds. The following moieties can be found in the linker: ether, thioether, carboxamide, thiourea, sulfonamide, urea, urethane, hydrazine, alkyl, aryl, heteroaryl, alkoxy, cycloalkyl and amine moieties. Examples of L include substituted or unsubstituted polymethylene, arylene, alkylarylene, arylenealkyl, or arylthio.

Any combination of linkers may be used to attach the reactive functional groups and the haptens together, typically a compound of the present invention when attached to more than one reactive functional group will have one or two linkers attached that may be the same or different. The linker may also be substituted to alter the physical properties of the present compounds, such as solubility and spectral properties of the compound.

III B. iii) Methods of Making the PI Met-Sensitive Moieties

The compounds of the invention are synthesized by an appropriate combination of generally well known synthetic methods. Techniques useful in synthesizing the compounds of the invention are both readily apparent and accessible to those of skill in the relevant art. The discussion below is offered to illustrate certain of the diverse methods available for use in assembling the compounds of the invention; it is not intended to define the scope of reactions or reaction sequences that are useful in preparing the compounds of the present invention.

In Schemes 1-5, preparatory schemes for haptens comprising met-sensitive moieties of amprenavir are presented.

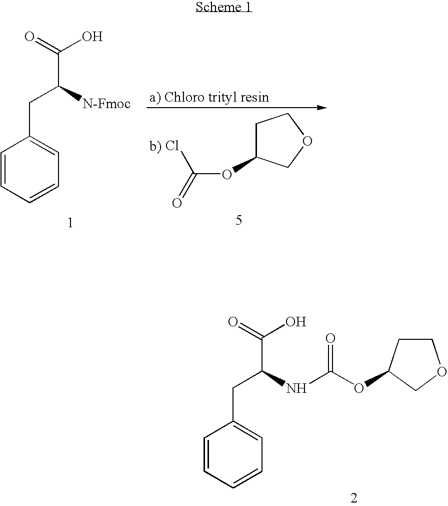

In Scheme 1, 1 is reacted with DIEA and chlorotrityl resin, and then 5, in order to form 2, which is a hapten comprising Met-Sensitive Moiety (A1).

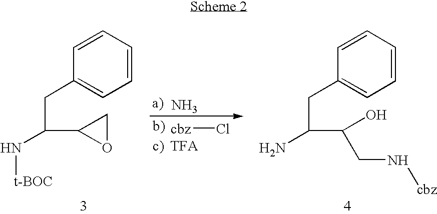

In Scheme 2, 3 is reacted with ammonia, then DIEA and benzylchloroformate, and finally trifluoroacetic acid in order to form 4.

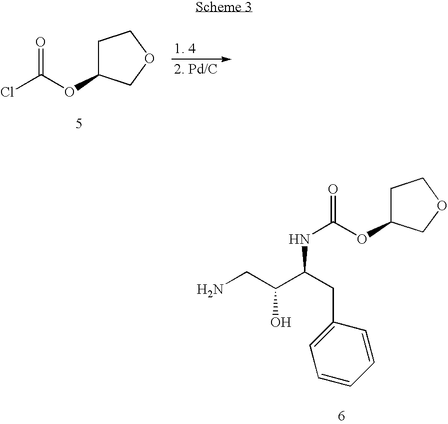

In Scheme 3, 5 is reacted with 4, then hydrogenated in order to form 6, which is a hapten comprising Met-Sensitive Moiety (A2).

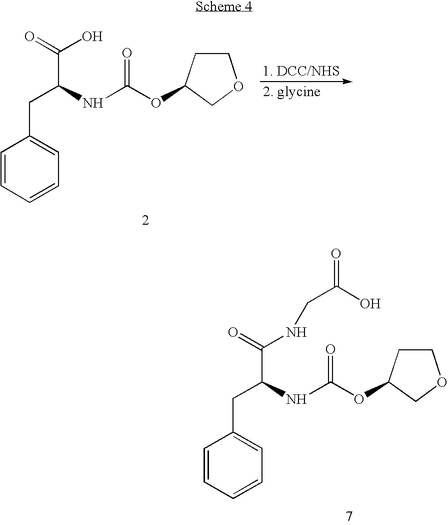

In Scheme 4, 2 is reacted with DCC, and then glycine, in order to form 7, which is a hapten comprising Met-Sensitive Moiety (A3).

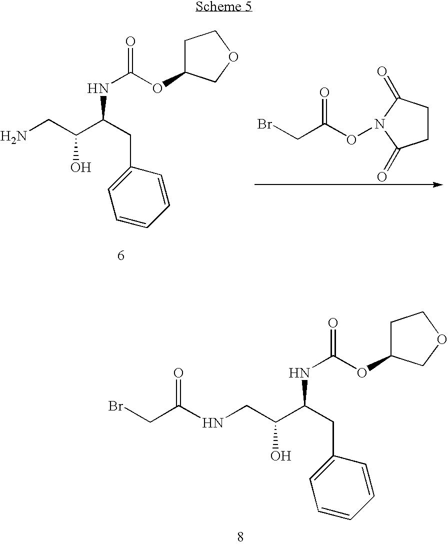

In Scheme 5, 6 is reacted with a bromoacetylated derivative, in order to form 8, which is a hapten comprising Met-Sensitive Moiety (A4).

In Schemes 6-10, preparatory schemes for haptens comprising met-sensitive moieties of atazanavir are presented.

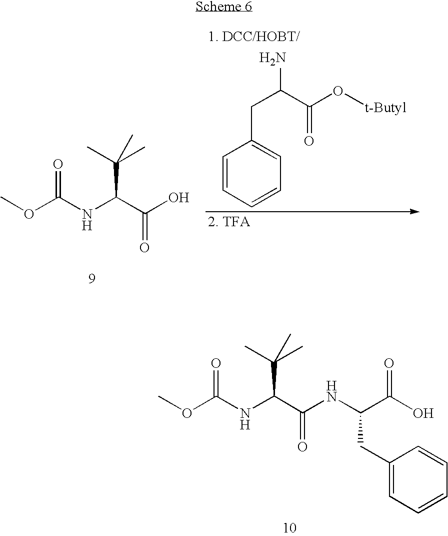

In Scheme 6, 9 is reacted with DCC and a phenylalanine derivative, and subsequently deprotected, in order to form 10, which is a hapten comprising Met-Sensitive Moiety (B1).

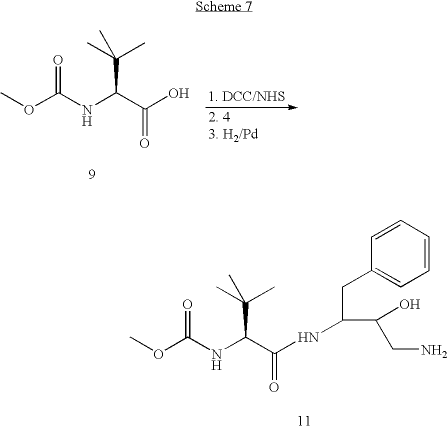

In Scheme 7, 9 is reacted with DCC and NHS, followed by 4, and subsequently hydrogenated to form 11, which is a hapten comprising Met-Sensitive Moiety (B2).

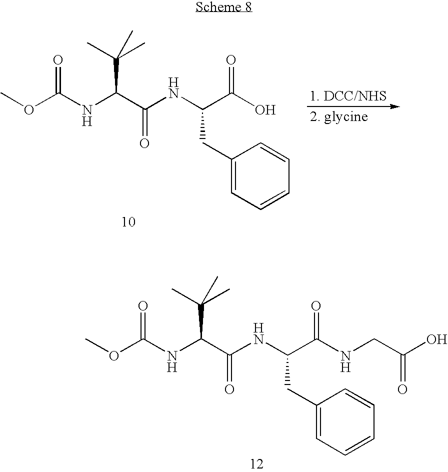

In Scheme 8, 10 is reacted with DCC and NHS, followed by glycine, in order to form 12, which is a hapten comprising Met-Sensitive Moiety (B3).

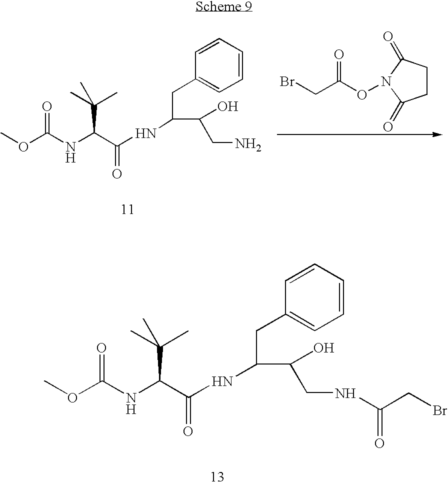

In Scheme 9, 11 is reacted with a bromoacetylated derivative, in order to form 13, which is a hapten comprising Met-Sensitive Moiety (B4).

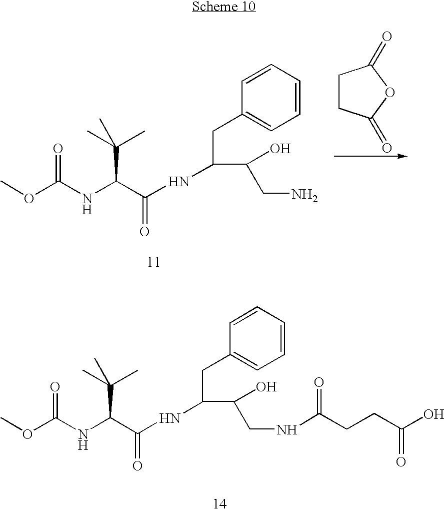

In Scheme 10, 11 is reacted with a succinyl anhydride in order to form 14, which is a hapten comprising Met-Sensitive Moiety (B5).

In Schemes 11-16, preparatory schemes for haptens comprising met-sensitive moieties of indinavir are presented.

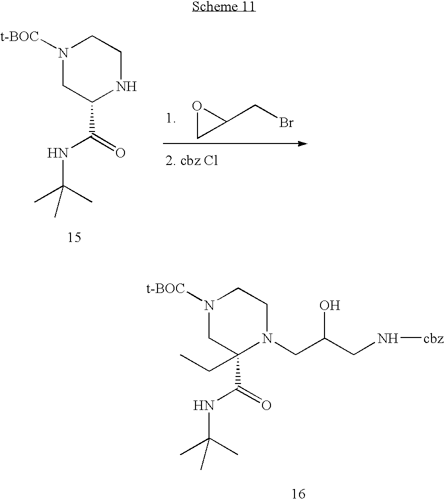

In Scheme 11, 15 is reacted with an oxirane, followed by ammonia and benzylchloroformate in order to form 16.

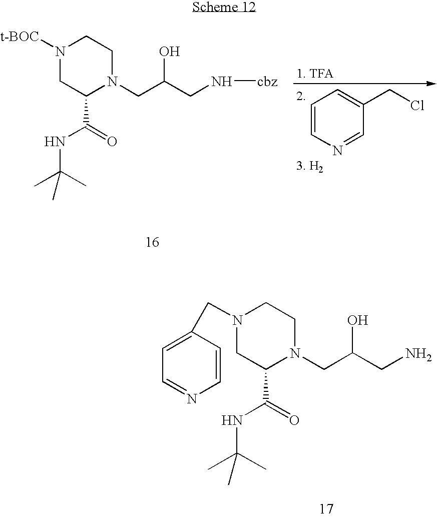

In Scheme 12, 16 is reacted with acid, chloromethylpyridine, and finally hydrogenated in order to form 17, which is a hapten comprising Met-Sensitive Moiety (C1).

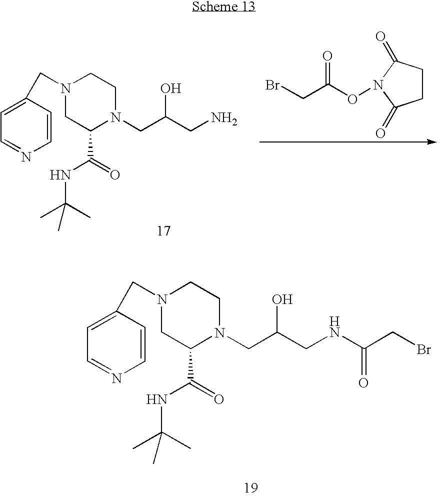

In Scheme 13, 17 is reacted with a bromoacetylated derivative, in order to form 19, which is a hapten comprising Met-Sensitive Moiety (C2).

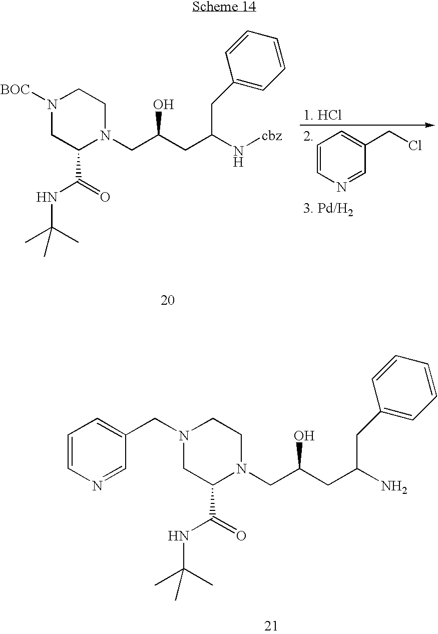

In Scheme 14, 20 is reacted with acid and chloromethylpyridine followed by Pd/H₂ in order to form 21, which is a hapten comprising Met-Sensitive Moiety (C3).

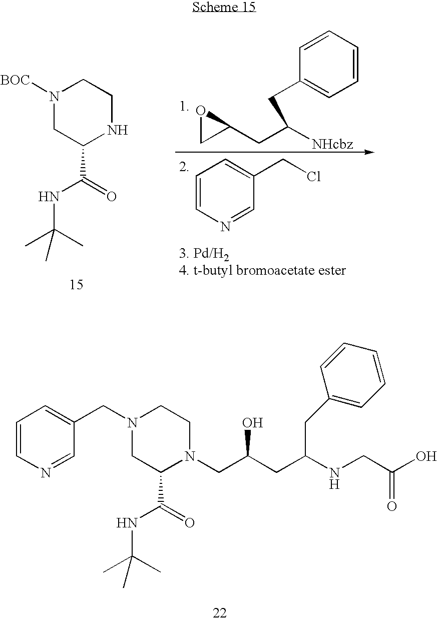

In Scheme 15, 15 is reacted with an oxirane acid and chloromethylpyridine followed by hydrogenation and reaction with bromoacetic acid derivative in order to form 22, which is a hapten comprising Met-Sensitive Moiety (C4).

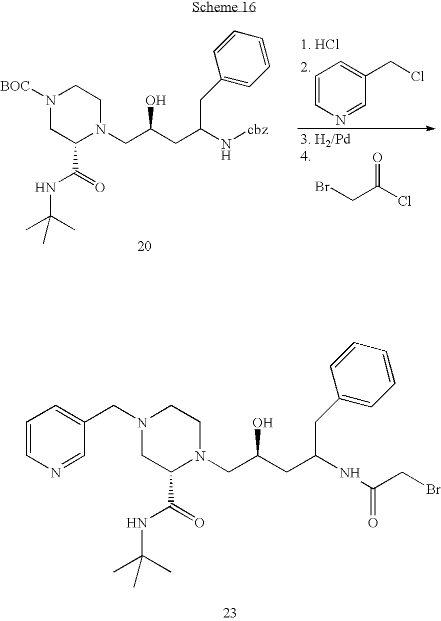

In Scheme 16, 20 is reacted with acid, chloromethylpyridine, followed by catalytic hydrogenation, and finally bromoacetic acid chloride in order to form 23, which is a hapten comprising Met-Sensitive Moiety (C5).

In Schemes 17-20, preparatory schemes for haptens comprising met-sensitive moieties of lopinavir are presented.

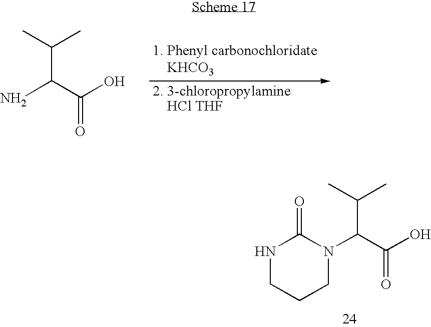

In Scheme 17, valine is reacted with phenyl carbonochloridate, and subsequently 3-chloropropylamine HCl in order to form 24, which is a hapten comprising Met-Sensitive Moiety (D1).

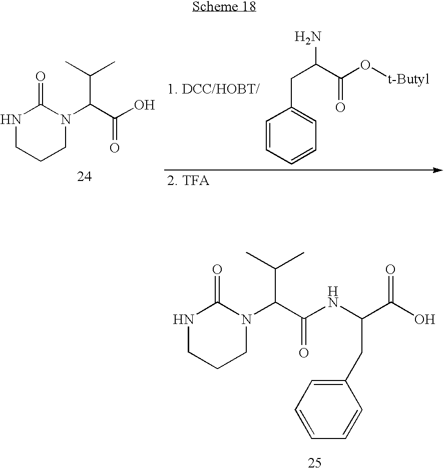

In Scheme 18, 24 is reacted with DCC and a phenylalanine derivative, and subsequently deprotected, in order to form 25, which is a hapten comprising Met-Sensitive Moiety (D2).

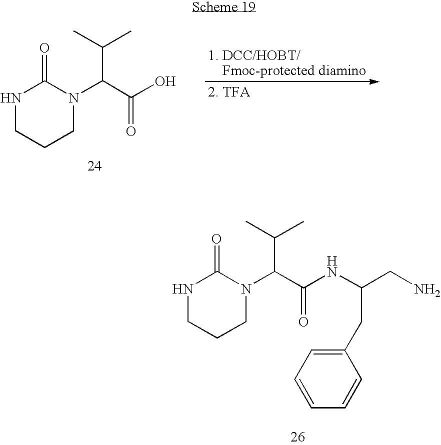

In Scheme 19, 24 is reacted with DCC and a Fmoc-protected diamino derivative, and subsequently deprotected, in order to form 26, which is a hapten comprising Met-Sensitive Moiety (D3).

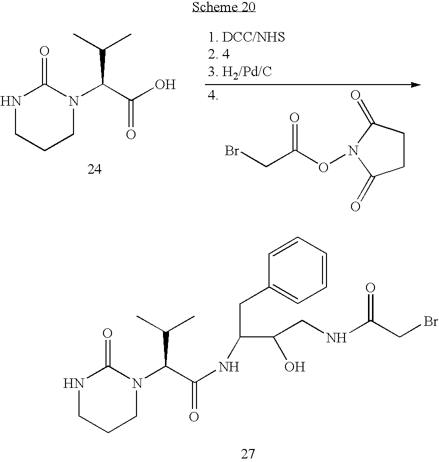

In Scheme 20, 24 is reacted with DCC and NHS, followed by 4, then hydrogenated, and finally reacted with a bromoacetylated derivative in order to form 27, which is a hapten comprising Met-Sensitive Moiety (D4).

In Schemes 21-27, preparatory schemes for haptens comprising met-sensitive moieties of nelfinavir are presented.

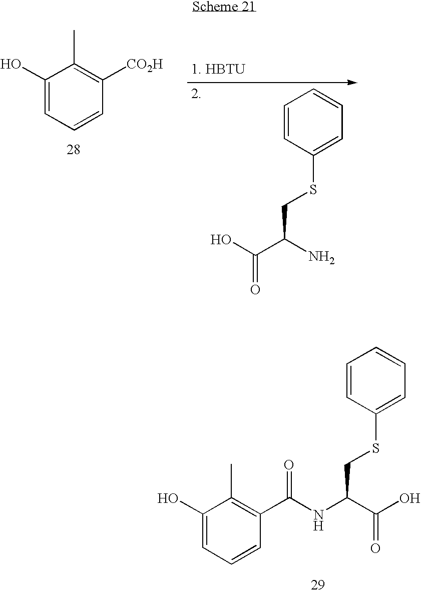

In Scheme 21, 28 is reacted with HBTU, followed by phenylthioylated amino acid derivative in order to form 29, which is a hapten comprising Met-Sensitive Moiety (E1).

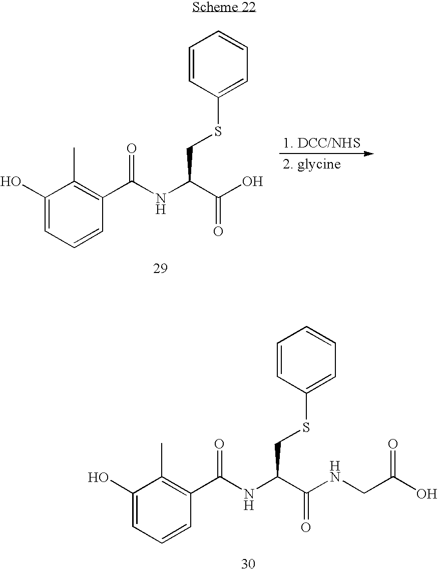

In Scheme 22, 29 is reacted with DCC and NHS, followed by glycine, in order to form 30, which is a hapten comprising Met-Sensitive Moiety (E2).

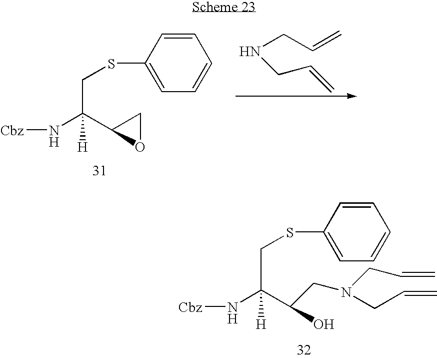

In Scheme 23, 31 is reacted with an amine in order to form 32.

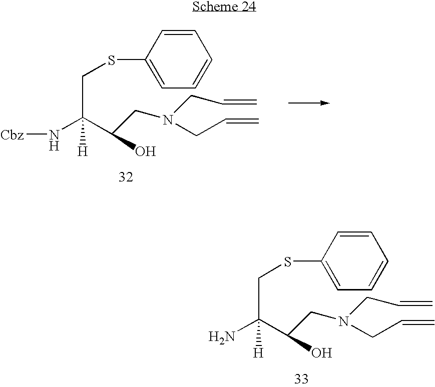

In Scheme 24, 32 is deprotected in order to form 33.

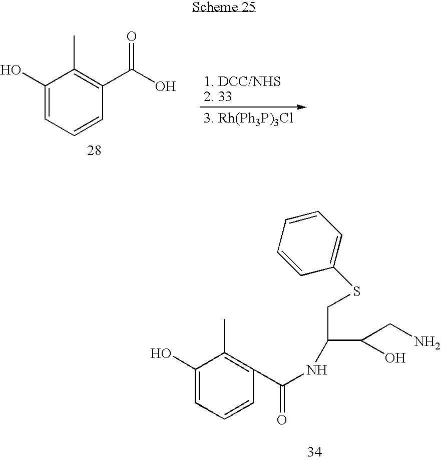

In Scheme 25, 28 is reacted with DCC and NHS, followed by 33 and deprotection in order to form 34, which is a hapten comprising Met-Sensitive Moiety (E3).

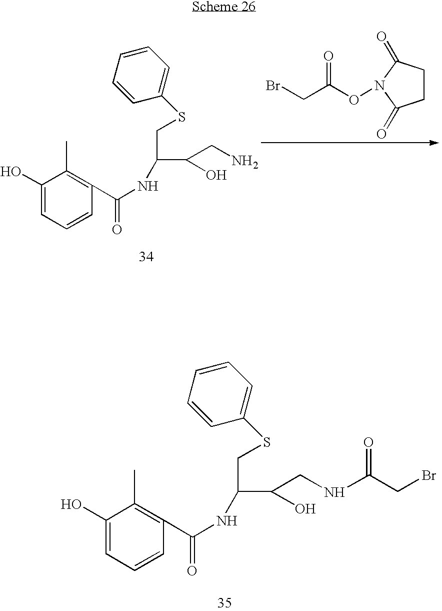

In Scheme 26, 34 is reacted with a bromoacetylated derivative in order to form 35, which is a hapten comprising Met-Sensitive Moiety (E4).

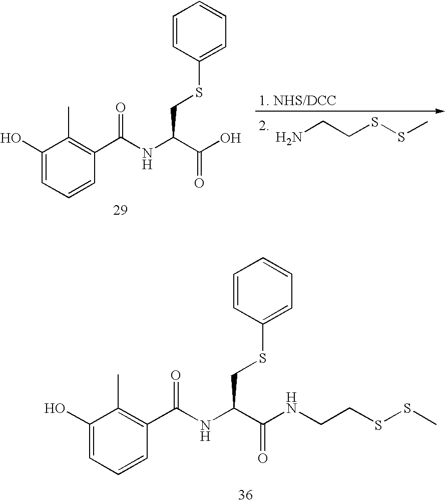

In Scheme 27, 29 is reacted with DCC and NHS, followed by a disulfidealkylamine derivative in order to form 36, which is a hapten comprising Met-Sensitive Moiety (E5).

Scheme 28 is a preparatory scheme for a hapten comprising met-sensitive moieties of ritonavir.

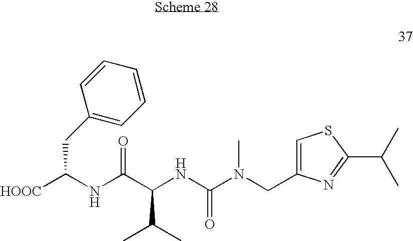

In Scheme 28, a valine derivative is reacted with DCC and NHS, followed by a reaction with phenylalanine derivative in order to form 37, which is a hapten comprising Met-Sensitive Moiety (F2).

In Schemes 29-33, preparatory schemes for haptens comprising met-sensitive moieties of saquinavir are presented.

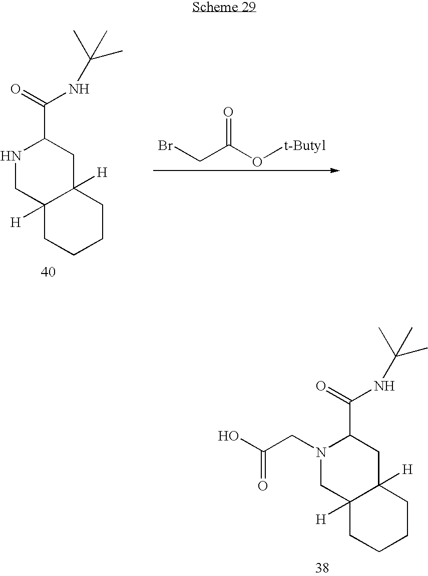

In Scheme 29, an isoquinoline derivative is reacted with bromoacetic acid bromine in order to form 38, which is a hapten comprising Met-Sensitive Moiety (G1).

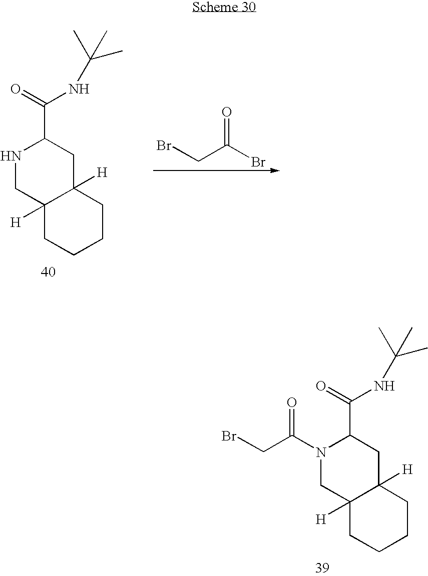

In Scheme 30, an isoquinoline derivative is reacted with bromoacetic acid bromine in order to form 39, which is a hapten comprising Met-Sensitive Moiety (G2).

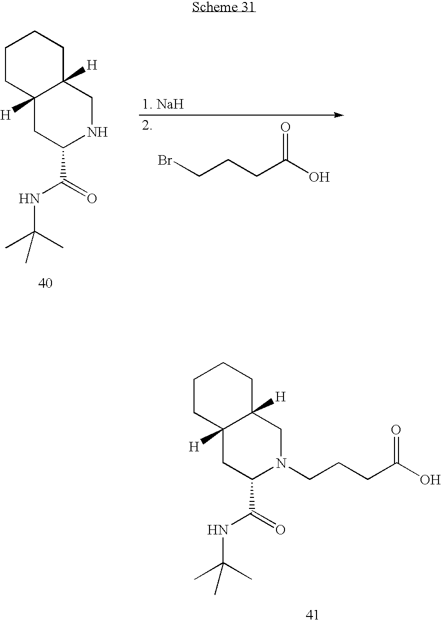

In Scheme 31, 40 is reacted with sodium hydride, followed by bromobutyric acid in order to form 41, which is a hapten comprising Met-Sensitive Moiety (G3).

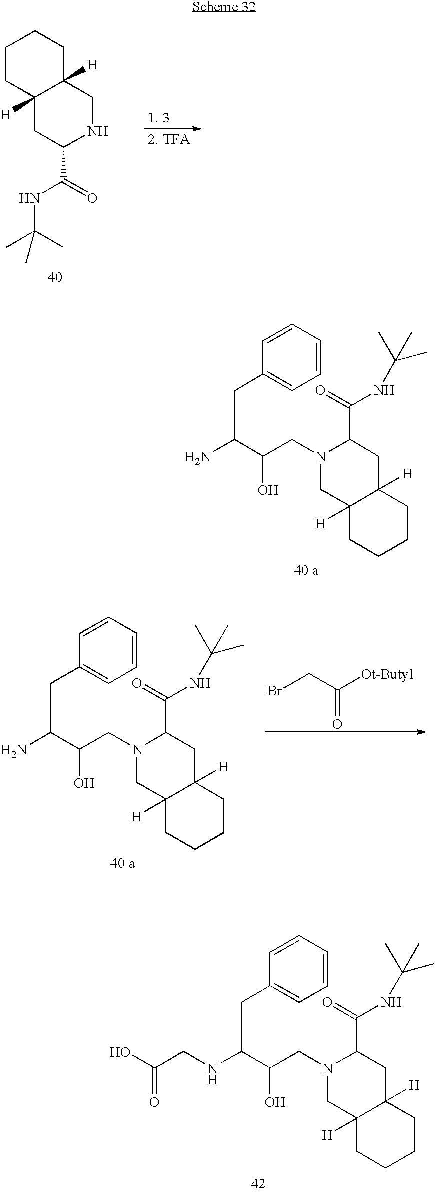

In Scheme 32, an isoquinoline derivative is reacted with 3 followed by acid and bromoacetyl derivative in order to form 42, which is a hapten comprising Met-Sensitive Moiety (G4).

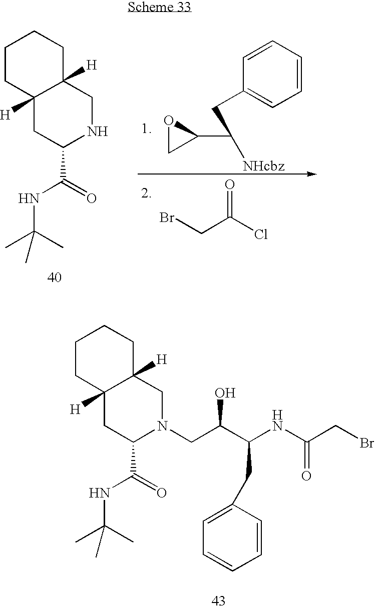

In Scheme 33, an isoquinoline derivative is reacted with an oxirane, followed by bromoacetic acid chloride in order to form 43, which is a hapten comprising Met-Sensitive Moiety (G5).

In Schemes 34-35, preparatory schemes for haptens comprising met-sensitive moieties of tipranavir are presented.

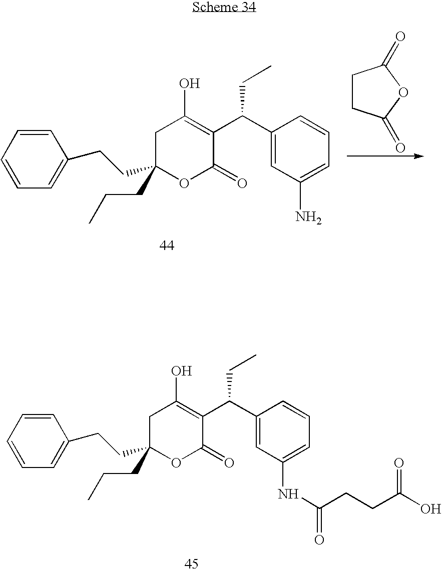

In Scheme 34, 44 is reacted with a succinyl anhydride in order to form 45, which is a hapten comprising Met-Sensitive Moiety (H1).

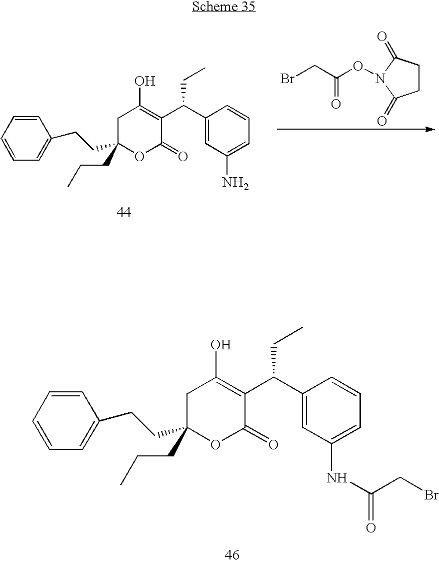

In Scheme 35, 44 is reacted with a bromoacetylated derivative in order to form 46, which is a hapten comprising Met-Sensitive Moiety (H2).

III. B. iv) Methods of Making the NNRTI Met-Sensitive Moieties

The compounds of the invention are synthesized by an appropriate combination of generally well known synthetic methods. Techniques useful in synthesizing the compounds of the invention are both readily apparent and accessible to those of skill in the relevant art. The discussion below is offered to illustrate certain of the diverse methods available for use in assembling the compounds of the invention; it is not intended to define the scope of reactions or reaction sequences that are useful in preparing the compounds of the present invention.

In Schemes 36-41, preparatory schemes for haptens comprising met-sensitive moieties of efavirenz are presented.

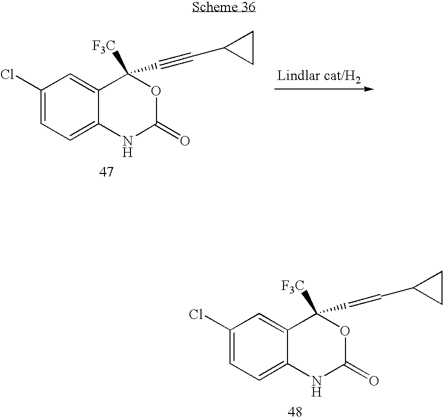

In Scheme 36, 47 is converted to a olefin derivative 48.

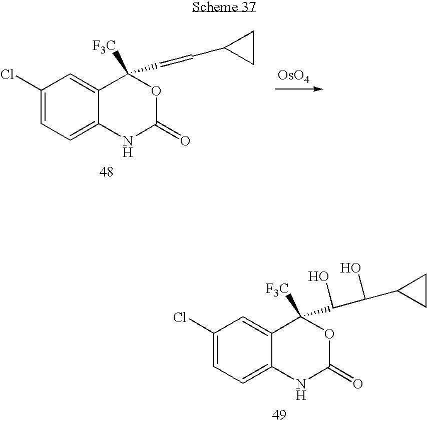

In Scheme 37, 48 is subjected to an oxidizing agent in order to form 49.

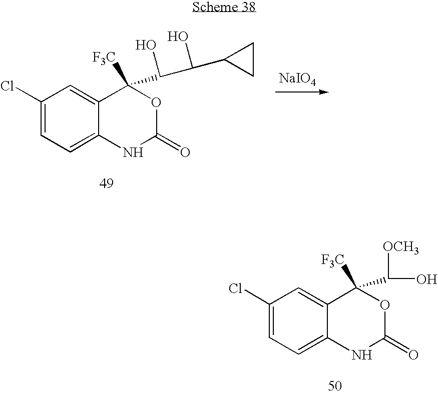

In Scheme 38, 49 is subjected to sodium periodate in order to form 50.

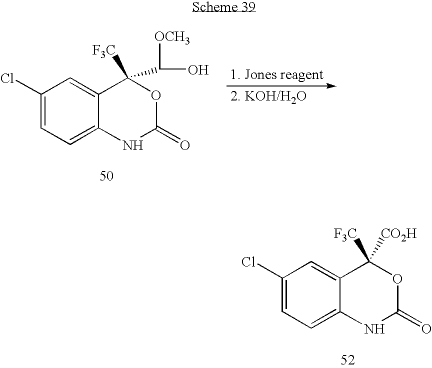

In Scheme 39, 50 is oxidized in order to form 52, which is a hapten comprising Met-Sensitive Moiety (11).

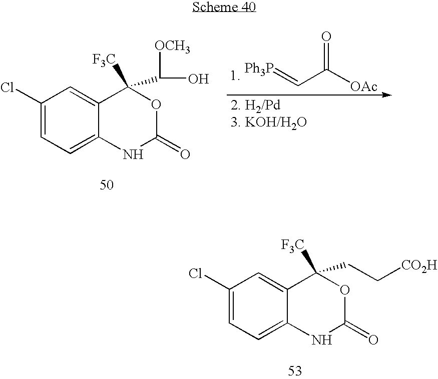

In Scheme 40, 50 is reacted with a triphenylphosphine derivative, then hydrogenated, and finally potassium hydroxide in order to form 53, which is a hapten comprising Met-Sensitive Moiety (I2).

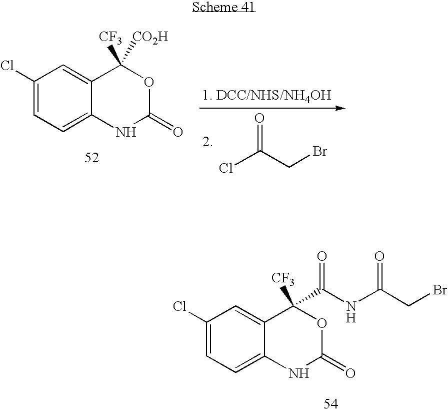

In Scheme 41, 52 is reacted with DCC, NHS and ammonium hydroxide, followed by a bromoacetylated derivative in order to form 54, which is a hapten comprising Met-Sensitive Moiety (I3).

III. B. v) Methods of Making the NNRTI Derivatives

The compounds of the invention are synthesized by an appropriate combination of generally well known synthetic methods. Techniques useful in synthesizing the compounds of the invention are both readily apparent and accessible to those of skill in the relevant art. The discussion below is offered to illustrate certain of the diverse methods available for use in assembling the compounds of the invention; it is not intended to define the scope of reactions or reaction sequences that are useful in preparing the compounds of the present invention.

In Schemes 42-43, preparatory schemes for haptens comprising NNRTI Derivatives of efavirenz are presented.

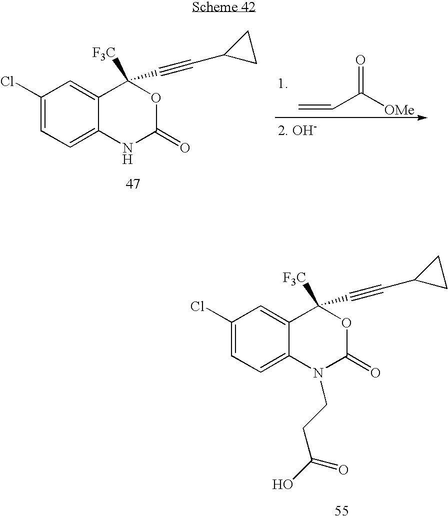

In Scheme 42, 47 is reacted with methyl 2-propenate, followed by a base in order to form 55, which is a hapten comprising NNRTI Derivative (I4).

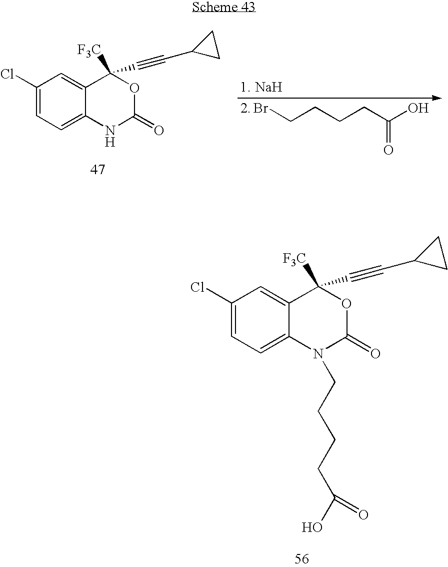

In Scheme 43, 47 is reacted with sodium hydride, followed by bromopentanoic acid in order to form 56, which is a hapten comprising NNRTI Derivative (I5).

In Schemes 44-45, preparatory schemes for haptens comprising NNRTI Derivatives of nevirapine are presented.

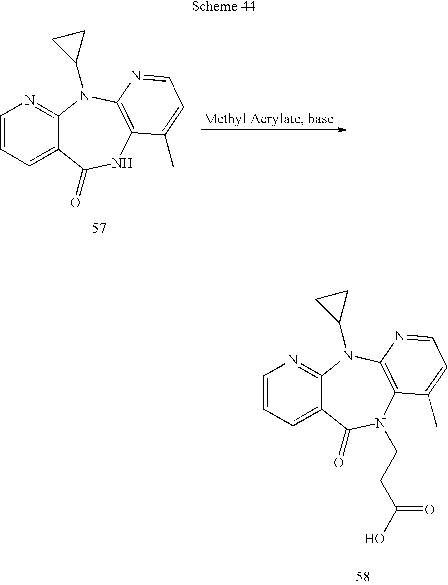

In Scheme 44, 57 is reacted with methyl acrylate and a base in order to form 58, which is a hapten comprising NNRTI Derivative (J1).

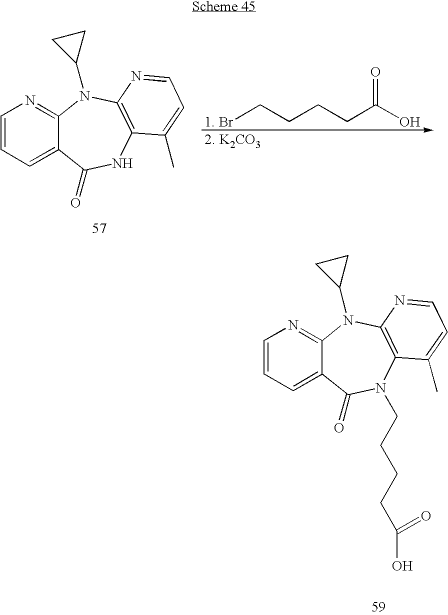

In Scheme 45, 57 is reacted with sodium hydride, followed by bromopentanoic acid in order to form 59, which is a hapten comprising NNRTI Derivative (J2).

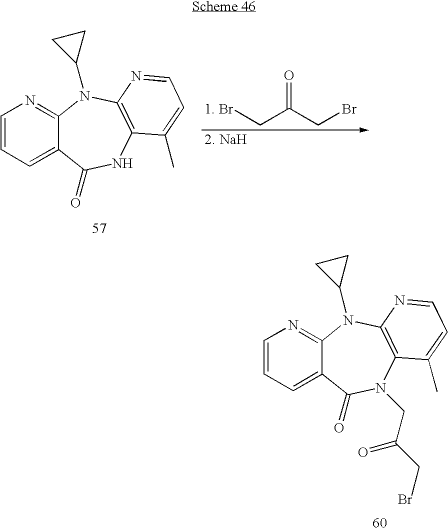

In Scheme 46, 57 is reacted with dibromoacetone and sodium hydride in order to form 60, which is a hapten comprising NNRTI Derivative (J3).

IV. Reactive Partners

The haptens comprising met-sensitive moieties or NNRTI Derivatives can be attached to one or more of a series of compounds known as reactive partners. The reactive partner can be an immunogenic carrier, a non-isotopic signal generating moiety, a solid support, one of a few miscellaneous types, or combinations thereof. It is possible for a compound to be a member of more than one reactive partner category. For example, an enzyme may be both a non-isotopic signal generating moiety, as well as an immunogenic carrier.

IV. A. Immunogenic Carriers: Creation of Immunogens or Met-Sensitive Antigens or NNRTI Derivative Antigens

The haptens comprising met-sensitive moieties or NNRTI Derivatives can be made immunogenic by coupling them to a suitable immunogenic carrier. This coupling produces a compound alternatively known as an immunogen, an antigen, a Met-Sensitive Antigen, or a NNRTI Derivative Antigen.

The immunogenic carrier may be attached to the compounds of the invention either directly through the met-sensitive moiety or NNRTI Derivative, or through a reactive functional group, if present, or through a non-isotpoic signal generating moiety, if present.

An immunogenic carrier is a group which, when conjugated to a met-sensitive moiety or NNRTI Derivative and injected into a mammal, will induce an immune response and elicit the production of antibodies that bind to the corresponding PI or NNRTI. Immunogenic carriers are also referred to as antigenic carriers and by other synonyms common in the art.

The molecular weight of immunogenic carriers typically range from about 2,000 to 10⁷, usually from about 20,060 to 600,000, and more usually from about 25,000 to 250,000 molecular weight. There will usually be at least about one met-sensitive moiety or NNRTI Derivative per 150,000 molecular weight, more usually at least one group per 50,000 molecular weight, preferably at least one group per 25,000 molecular weight.

Various protein types may be employed as the poly(amino acid) immunogenic carrier. These types include albumins, serum proteins, e.g., globulins, ocular lens proteins, lipoproteins, etc. Illustrative proteins include bovine serum albumin (BSA), keyhole limpet hemocyanin (KLH), egg ovalbumin, bovine gamma-globulin (BGG), etc. Alternatively, synthetic poly(amino acids) may be utilized.

The immunogenic carrier can also be a polysaccharide, which is a high molecular weight polymer built up by repeated condensations of monosaccharides. Examples of polysaccharides are starches, glycogen, cellulose, carbohydrate gums, such as gum arabic, agar, and so forth. The polysaccharide can also contain polyamino acid residues and/or lipid residues.

The immunogenic carrier can also be a poly(nucleic acid) either alone or conjugated to one of the above mentioned poly(amino acids) or polysaccharides.

The immunogenic carrier can also be a particle. The particles are generally at least about 0.02 microns and not more than about 100 microns, usually at least about 0.05 microns and less than about 20 microns, preferably from about 0.3 to 10 microns diameter. The particle may be organic or inorganic, swellable or non-swellable, porous or non-porous, preferably of a density approximating water, generally from about 0.7 to 1.5 g/mL, and composed of material that can be transparent, partially transparent, or opaque. The particles can be biological materials such as cells and microorganisms, e.g., erythrocytes, leukocytes, lymphocytes, hybridomas, Streptococcus, Staphylococcus aureus, E. coli, viruses, and the like. The particles can also comprise organic and inorganic polymers, liposomes, latex particles, phospholipid vesicles, chylomicrons, lipoproteins, and the like.

The polymers can be either addition or condensation polymers. Particles derived therefrom will be readily dispersible in an aqueous medium and may be adsorptive or functionalizable so as to bind (conjugate) to a met-sensitive moiety or NNRTI Derivative of the invention.

The particles can be derived from naturally occurring materials, naturally occurring materials which are synthetically modified, and synthetic materials. Among organic polymers of particular interest are polysaccharides, particularly cross-linked polysaccharides, such a agarose, which is available as Sepharose, dextran, available as Sephadex and Sephacryl, cellulose, starch, and the like; addition polymers, such as polystyrene, polyvinyl alcohol, homopolymers and copolymers of derivatives of acrylate and methacrylate, particularly esters and amides having free hydroxyl functionalities, and the like.

The particles will usually be polyfunctional and will be bound to or be capable of binding (being conjugated) to a met-sensitive moiety or NNRTI Derivative. Descriptions of the binding of the particles to the met-sensitive moieties or NNRTI Derivatives are provided in Section III.

IV B. Non-Isotopic Signal Generating Moiety

In the methods and compositions of this invention, a variety of signal-generating moieties can be employed. Among these moieties are fluorophores, chemiluminescent compounds, enzymes, inorganic particles, magnetic beads, and colloidal gold. The non-isotopic signal generating moieties discussed herein can be attached to the haptens comprising the met-sensitive moieties or NNRTI Derivatives according to the methods described in Section III and Example 40-43. One of skill in the art would appreciate that non-isotopic signal generating moieties appropriate for the invention but not explicitly referenced in this document can be found in a textbooks or catalogs, such as Handbook of Fluorescent Probes and Research Products, 9th ed., Richard Haugland, ed. (Molecular Probes, 2003), which is herein incorporated by reference. Chapter 7 of the Handbook is especially useful for selecting non-isotopic signal generating moieties that are appropriate for use in the invention.

The non-isotopic signal-generating moiety may be attached to the compounds of the invention either directly through the met-sensitive moiety or NNRTI Derivative, or through a reactive functional group, if present, or through an immunogenic carrier, if present. Non-isotopic signal generating moieties may also be attached to receptors of the invention, as described elsewhere herein. Finally, the non-isotopic signal generating moieties discussed herein can be utilized in the immunoassays and kits of the invention.

IV. B. i) Fluorophores

For the purposes of the invention a fluorophore can be a substance which itself fluoresces, can be made to fluoresce, or can be a fluorescent analogue of an analyte.

In principle, any fluorophore can be used in the assays of this invention. Preferred fluorophores, however, have the following characteristics:
a. A fluorescence lifetime of greater than about 15 nsec;
b. An excitation wavelength of greater than about 350 nm;
c. A Stokes shift (a shift to lower wave-length of the emission relative to absorption) of greater than about 20 nm;
d. For homogeneous assays, fluorescence lifetime should vary with binding status; and
e. The absorptivity and quantum yield of the fluorophore should be high.

The longer lifetime is advantageous because it is easier to measure and more easily distinguishable from the Raleigh scattering (background). Excitation wavelengths greater than 350 nm reduce background interference because most fluorescent substances responsible for background fluorescence in biological samples are excited below 350 nm. A greater Stokes shift also allows for less background interference.

The fluorophore should have a functional group available for conjugation either directly or indirectly to the Met-Sensitive antigen, NNRTI Derivative antigen, or receptor. An additional criterion in selecting the fluorophore is the stability of the fluorophore: it should not be photophysically unstable, and it should be relatively insensitive to the assay conditions, e.g., pH, polarity, temperature and ionic strength.

Preferably (though not necessarily), fluorophores for use in heterogenous assays are relatively insensitive to binding status. In contrast, fluorophores for use in homogeneous assay must be sensitive to binding status, i.e., the fluorescence lifetime must be alterable by binding so that bound and free forms can be distinguished.

Examples of fluorophores useful in the invention are naphthalene derivatives (e.g. dansyl chloride), anthracene derivatives (e.g. N-hydroxysuccinimide ester of anthracene propionate), pyrene derivatives (e.g. N-hydroxysuccinimide ester of pyrene butyrate), fluorescein derivatives (e.g. fluorescein isothiocyanate), rhodamine derivatives (e.g. rhodamine isothiocyanate), phycoerythin, and Texas Red.

IV. B ii) Enzymes

In an exemplary embodiment, the non-isotopic signal generating moiety is an enzyme. From the standpoint of operability, a very wide variety of enzymes can be used. But, as a practical matter, some enzymes have characteristics which make them preferred over others. The enzyme should be stable when stored for a period of at least three months, and preferably at least six months at temperatures which are convenient to store in the laboratory, normally −20° C. or above. The enzyme should also have a satisfactory turnover rate at or near the pH optimum for binding to the receptor, this is normally at about pH 6-10, usually 6.0 to 8.0. A product should be either formed or destroyed as a result of the enzyme reaction which absorbs light in the ultraviolet region or the visible region, that is the range of about 250-750 nm., preferably 300-600 nm. The enzyme also should have a substrate (including cofactors) which has a molecular weight in excess of 300, preferably in excess of 500, there being no upper limit. The enzyme which is employed or other enzymes, with like activity, will not be present in the sample to be measured, or can be easily removed or deactivated prior to the addition of the assay reagents. Also, there should not be naturally occurring inhibitors for the enzyme present in fluids to be assayed.

Also, although enzymes of up to 600,000 molecular weight can be employed, usually relatively low molecular weight enzymes will be employed of from 10,000 to 300,000 molecular weight, more usually from about 10,000 to 150,000 molecular weight, and frequently from 10,000 to 100,000 molecular weight. Where an enzyme has a plurality of subunits the molecular weight limitations refer to the enzyme and not to the subunits.

For synthetic convenience, it is preferable that there be a reasonable number of groups to which the met-sensitive antigen, NNRTI Derivative antigen, or receptor may be bonded, particularly amino groups. However, other groups to which the met-sensitive antigen, NNRTI Derivative antigen or antibody may be bonded include hydroxyl groups, thiols, and activated aromatic rings, e.g., phenolic.

Finally, for the purposes of this invention, the enzymes should be capable of specific labeling so as to be useful in the subject assays. Specific labeling means attachment at a site related to the active site of the enzyme, so that upon binding of the receptor (met-sensitive antigen, NNRTI Derivative antigen or receptor, depending on the specific immunoassay) to the ligand (again, either the met-sensitive antigen, NNRTI Derivative antigen, or receptors), the enzyme is satisfactorily enhanced or inhibited.

Based on these criteria, the following enzymes can be used in the invention: alkaline phosphatase, horseradish peroxidase, lysozyme, glucose-6-phosphate dehydrogenase, lactate dehydrogenase, β-galactosidase, and urease. Also, a genetically engineered fragment of an enzyme may be used, such as the donor and acceptor fragment of β-galactosidase utilized in CEDIA immunoassays (see Henderson D R et al. Clin Chem. 32(9): 1637-1641 (1986)); U.S. Pat. No. 4,708,929. These and other enzymes which can be used have been discussed in detail by Eva Engvall in Enzyme Immunoassay ELISA and EMIT in Methods in Enzymology, 70:419-439 (1980) and in U.S. Pat. No. 4,857,453.

Enzymes, enzyme fragments, enzyme inhibitors, enzyme substrates, and other components of enzyme reaction systems can be attached to the haptens and receptors, and employed in the immunoassays of the invention. Where any of these components is used as a non-isotopic signal generating moiety, a chemical reaction involving one of the components is part of the signal producing system.

Coupled catalysts can also involve an enzyme with a non-enzymatic catalyst. The enzyme can produce a reactant, which undergoes a reaction catalyzed by the non-enzymatic catalyst or the non-enzymatic catalyst may produce a substrate (includes coenzymes) for the enzyme. A wide variety of non-enzymatic catalysts, which may be employed are found in U.S. Pat. No. 4,160,645 (1979), the appropriate portions of which are incorporated herein by reference.

The enzyme or coenzyme employed provides the desired amplification by producing a product which absorbs light, e.g., a dye, or emits light upon irradiation, e.g., a fluorescer. Alternatively, the catalytic reaction can lead to direct light emission, e.g., chemiluminescence. A large number of enzymes and coenzymes for providing such products are indicated in U.S. Pat. No. 4,275,149, columns 19 to 23, and U.S. Pat. No. 4,318,980, columns 10 to 14, which disclosures are incorporated herein by reference.

A number of enzyme combinations are set forth in U.S. Pat. No. 4,275,149, columns 23 to 28, which combinations can find use in the subject invention. This disclosure is incorporated herein by reference.

When a single enzyme is used as a label, such enzymes that may find use are hydrolases, transferases, lyases, isomerases, ligases or synthetases and oxidoreductases. In an exemplary embodiment, the enzyme is a hydrolase. Alternatively, luciferases may be used such as firefly luciferase and bacterial luciferase. Illustrative dehydrogenases include malate dehydrogenase, glucose-6-phosphate dehydrogenase, and lactate dehydrogenase. Illustrative oxidases include glucose oxidase. Of the peroxidases, horse radish peroxidase is illustrative. Of the hydrolases, alkaline phosphatase, β-glucosidase and lysozyme are illustrative.

Of particular interest are enzymes which involve the production of hydrogen peroxide and the use of the hydrogen peroxide to oxidize a dye precursor to a dye. Particular combinations include saccharide oxidases, e.g., glucose and galactose oxidase, or heterocyclic oxidases, such as uricase and xanthine oxidase, coupled with an enzyme which employs the hydrogen peroxide to oxidize a dye precursor, that is, a peroxidase such as horse radish peroxidase, lactoperoxidase, or microperoxidase. Additional enzyme combinations may be found in the subject matter incorporated by reference.

Those enzymes, which employ nicotinamide adenine dinucleotide (NAD) or its phosphate (NADP) as a cofactor, particularly the former, can be used. One preferred enzyme is glucose-6-phosphate dehydrogenase, preferably, NAD-dependent glucose-6-phosphate dehydrogenase.

IV. B. iii) Colloidal Gold

In an exemplary embodiment, the hapten-reactive partner conjugates, as well as the receptors of the invention can comprise a colloidal gold moiety. The immunoassays of the invention can also comprise a colloidal gold moiety. A colloidal gold moiety may possess any chosen size from 1-250 nm. This gold probe detection system, when incubated with a specific target, such as in an immunoassay, will reveal the target through the visibility of the gold particles themselves. The gold particles can be detected by a variety of methods, such as by microscope or eye. Visibility can be enhanced through a short and simple silver enhancing procedure. For detection by eye, gold particles will also reveal immobilized protein on a solid phase such as a blotting membrane through the accumulated red color of the gold. Silver enhancement of this gold precipitate also gives further sensitivity of detection. Further information about colloidal gold can be found in Handbook of Fluorescent Probes and Research Products, 9^{th} ed., Richard Haugland, ed. (Molecular Probes, 2003), specifically in chapter 7, p. 251-254.

IV. C. Solid Support

In an exemplary embodiment, a reactive partner for the compounds of the invention is a solid support. The solid support may be attached to the compound either directly through the met-sensitive moiety or NNRTI Derivative, or through the reactive functional group, if present, or through an immunogenic carrier molecule, if present. Even if a reactive functional group and/or an immunogenic carrier are present, the solid support may be attached through the met-sensitive moiety or NNRTI Derivative.

A solid support suitable for use in the present invention is typically substantially insoluble in liquid phases. Solid supports of the current invention are not limited to a specific type of support. Rather, a large number of supports are available and are known to one of ordinary skill in the art. Thus, useful solid supports include semi-solids, such as aerogels and hydrogels, resins, beads, biochips (including thin film coated biochips), multi-well plates (also referred to as microtiter plates), membranes, conducting and nonconducting metals and magnetic supports. More specific examples of useful solid supports include silica gels, polymeric membranes, particles, derivatized plastic films, glass beads, cotton, plastic beads, alumina gels, polysaccharides such as Sepharose, poly(acrylate), polystyrene, poly(acrylamide), polyol, agarose, agar, cellulose, dextran, starch, FICOLL, heparin, glycogen, amylopectin, mannan, inulin, nitrocellulose, diazocellulose, polyvinylchloride, polypropylene, polyethylene (including poly(ethylene glycol)), nylon, latex bead, magnetic bead, paramagnetic bead, superparamagnetic bead, starch and the like.

In some embodiments, the solid support may include a solid support reactive functional group, including, but not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, etc., for attaching the compounds of the invention. Useful reactive groups are disclosed below and are equally applicable to the solid support reactive functional groups herein.

A suitable solid phase support can be selected on the basis of desired end use and suitability for various synthetic protocols. For example, where amide bond formation is desirable to attach the compounds of the invention to the solid support, resins generally useful in peptide synthesis may be employed, such as polystyrene (e.g., PAM-resin obtained from Bachem Inc., Peninsula Laboratories, etc.), POLYHIPE™ resin (obtained from Aminotech, Canada), polyamide resin (obtained from Peninsula Laboratories), polystyrene resin grafted with polyethylene glycol (TentaGel™, Rapp Polymere, Tubingen, Germany), polydimethyl-acrylamide resin (available from Milligen/Biosearch, California), or PEGA beads (obtained from Polymer Laboratories).

IV. D. Miscellaneous

Miscellanoues reactive partners of the invention include a polypeptide, polysaccharide, a synthetic polymer, and combinations thereof.

IV. E. Methods of Attaching a Hapten to a Reactive Partner

There are many options available for the conjugation of a hapten comprising a met-sensitive moiety or a NNRTI Derivative with a reactive partner. In an exemplary embodiment, the hapten comprises a reactive functional group, and is conjugated to the reactive partner. An illustration of this strategy is provided in Example 40 and 43. In another exemplary embodiment, the reactive partner is activated, and then conjugated to the compound comprising the met-sensitive moiety. Illustrations of this strategy are provided in Examples 41 and 42. These conjugations produce a hapten-reactive partner conjugate

The methods of attaching are dependent upon the reactive groups present at the site of activation. In an exemplary embodiment, the reactive functional group of the haptens of the invention and the functional group of the reactive part comprise electrophiles and nucleophiles that can generate a covalent linkage between them. Alternatively, the reactive functional group comprises a photoactivatable group, which becomes chemically reactive only after illumination with light of an appropriate wavelength. Typically, the conjugation reaction between the reactive functional group and the reactive partner results in one or more atoms of the reactive functional group or the reactive partner being incorporated into a new linkage attaching the hapten to the reactive partner. Selected examples of functional groups and linkages are shown in Table 1, where the reaction of an electrophilic group and a nucleophilic group yields a covalent linkage.

TABLE 1
Examples of some routes to useful covalent linkages
with electrophile and nucleophile reactive groups

| Electrophilic Group | Nucleophilic Group | Resulting Covalent Linkage |
|---|---|---|
| activated esters* | amines/anilines | carboxamides |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carboxylic acids | amines/anilines | carboxamides |
| carboxylic acids | alcohols | esters |
| carboxylic acids | hydrazines | hydrazides |
| carbodiimides | carboxylic acids | N-acylureas or anhydrides |
| diazoalkanes | carboxylic acids | esters |
| epoxides | thiols | thioethers |
| haloacetamides | thiols | thioethers |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |
| *Activated esters, as understood in the art, generally have the formula —COΩ, where Ω is a good leaving group (e.g. oxysuccinimidyl(—OC₄H₄O₂)oxysulfosuccinimidyl(—OC₄H₃O₂—SO₃H),-1-oxybenzotriazolyl(—OC₆H₄N₃); or an aryloxy group or aryloxy substituted one or more times by electron withdrawing substituents such as nitro, | | |
| # fluoro, chloro, cyano, or trifluoromethyl, or combinations thereof, used to form activated aryl esters; or a carboxylic acid activated by a carbodiimide to form an anhydride or mixed anhydride —OCOR^{a} or —OCNR^{a}NHR^{b}, where R^{a} and R^{b}, which may be the same or different, are C₁-C₆ alkyl, C₁-C₆ perfluoroalkyl, or C₁-C₆ alkoxy; or cyclohexyl, 3-dimethylaminopropyl, or N-morpholinoethyl). | | |

**Acyl azides can also rearrange to isocyanates

Where the reactive functional group is an activated ester of a carboxylic acid, such as a succinimidyl ester of a carboxylic acid, the resulting compound is particularly useful for preparing conjugates of carrier molecules such as proteins, nucleotides, oligonucleotides, or haptens. Where the reactive group is a maleimide or haloacetamide the resulting compound is particularly useful for conjugation to thiol-containing substances. Where the reactive group is a hydrazide, the resulting compound is particularly useful for conjugation to periodate-oxidized carbohydrates and glycoproteins, and in addition is an aldehyde-fixable polar tracer for cell microinjection. Where the reactive group is a silyl halide, the resulting compound is particularly useful for conjugation to silica surfaces, particularly where the silica surface is incorporated into a fiber optic probe subsequently used for remote ion detection or quantitation.

In order to conjugate haptens comprising met-sensitive moieties or NNRTI Derivatives to a reactive partner, the haptens comprising the met-sensitive moieties and NNRTI Derivatives are typically first dissolved in water or a water-miscible such as a lower alcohol, dimethylformamide (DMF), dimethylsulfoxide (DMSO), acetone, acetonitrile, tetrahydrofuran (THF), dioxane or acetonitrile. These methods are been described in detail in Hermanson Greg T., Bioconjugate Techniques, Chapter 9, p. 419-455, Academic Press, Inc., 1996, which is incorporated herein by reference. Conjugates typically result from mixing appropriate reactive compounds and the component to be conjugated in a suitable solvent in which both are soluble, using methods well known in the art, followed by separation of the conjugate from any unreacted component and by-products. These present compounds are typically combined with the component under conditions of concentration, stoichiometry, pH, temperature and other factors that affect chemical reactions that are determined by both the reactive groups on the compound and the expected site of modification on the component to be modified. These factors are generally well known in the art of forming bioconjugates (Haugland et al., “Coupling of Antibodies with Biotin”, The Protein Protocols Handbook, J. M. Walker, ed., Humana Press, (1996); Haugland “Coupling of Monoclonal Antibodies with Fluorophores”, Methods in Molecular Biology, Vol. 45: Monoclonal Antibody Protocols, W. C. Davis, ed. (1995)). For those reactive compounds that are photoactivated, conjugation requires illumination of the reaction mixture to activate the reactive compound. The labeled component is used in solution or lyophilized and stored for later use.

IV. E. i) Methods of Attaching a Colloidal Gold Moiety

The conjugation of selected proteins to gold particles depends upon at least three physical phenomena. The first is the charge attraction of the negative gold particle to positively charged protein, receptor, solid support, or hapten. The second is the hydrophobic absorption of the protein, receptor, solid support, or hapten to the gold particle surface. The third is the binding of the gold to sulphur (dative binding) where this may exist within the structure of the protein, receptor, solid support, or hapten.

V. Receptors

V. A. Introduction

Included within the invention are receptors specific for the Met-Sensitive Moieties or NNRTI Derivatives described within. Also included within the invention are receptors that substantially compete with the binding of the receptors specific for the Met-Sensitive Moieties or NNRTI Derivatives described within. In an exemplary embodiment, the receptor is an antibody. In another exemplary embodiment, the receptor comprises the antigen-binding residues of an antibody. In another exemplary embodiment, the receptor can further comprise a non-isotopic signal generating moiety as discussed herein. The methods of attaching the non-isotopic signal generating moieties to the haptens of the invention are applicable to the methods of attaching the non-isotopic signal generating moieties to the receptors of the invention.

B. Antibodies

Antibodies, or immunoglobulins, are molecules produced by organs of the immune system to defend against antigens. The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Cellular and Molecular Immunology Ch. 3 (Abbas and Lichtman, ed., 5th ed. Saunders (2003)) (incorporated by reference in its entirety for all purposes). The variable regions of each light/heavy chain pair form the antibody binding site. Thus, an intact IgG antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

Antibodies exist as intact immunoglobulins or as a number of well-characterized fragments. Basic antibody fragments include Fab, which consists of portions of a heavy chain (above the hinge region) and a light chain, and Fab′, which is essentially Fab with part of the hinge region attached. Peptidases digest the antibody in different ways to produce fragments with combinations of these basic antibody fragments. Thus, for example, pepsin digests an antibody below the disulfide linkages in the hinge region to produce F(ab)′_{2,} a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into a Fab′ monomer. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments.

V. B. i) Production of Antibodies

Antibodies specific for the antigens of the invention may be produced by in vitro or in vivo techniques. In vitro techniques involve exposure of lymphocytes to the met-sensitive antigens or NNRTI Derivative antigens, while in vivo techniques, such as the production of polyclonal and monoclonal antibodies, require the injection of the met-sensitive antigens or NNRTI Derivative antigens into a suitable vertebrate host.

Polyclonal antibody production methods are known to those of skill in the art and can be conducted on suitable vertebrate hosts, including mice, rats, rabbits, sheep, goats, and the like. In an exemplary embodiment, an inbred strain of mice (e.g., BALB/C mice) or rabbits is injected with the met-sensitive antigen or NNRTI Derivative antigen using a standard adjuvant, such as Freund's adjuvant, according to a standard immunization protocol. The injections may be made intramuscularly, intraperitoneally, subcutaneously, or the like. The animal's immune response to the met-sensitive antigen or NNRTI Derivative antigen preparation is monitored by taking test bleeds and determining the titer of reactivity to the met-sensitive antigen. When appropriately high titers of antibody to the met-sensitive antigen or NNRTI Derivative antigen are obtained, blood is collected from the animal and antisera are prepared. Further fractionation of the antisera to enrich for antibodies reactive to the met-sensitive antigen or NNRTI Derivative antigen or anti-HIV therapeutic can be done if desired (see, Harlow & Lane, supra).

Monoclonal antibodies may be obtained by various techniques familiar to those skilled in the art. Briefly, spleen cells from an animal injected with a met-sensitive antigen or NNRTI Derivative antigen are immortalized, commonly by fusion with a myeloma cell (see, Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the met-sensitive antigen or NNRTI Derivative antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246:1275-1281 (1989).

V. B. ii) Screening for Antibodies

Monoclonal antibodies and polyclonal sera are collected and titered against the met-sensitive antigens or NNRTI Derivative antigens of the invention in an immunoassay, which is described in Section VI below. Specifically, for monoclonal antibodies the selection methods are divided into a primary and secondary screening method. In the case of polyclonal sera only the secondary screening method is used.

The primary screening method is a reverse ELISA procedure which was set up such that the monoclonal antibody is bound on the Enzyme Immunoassay (EIA) plate by rabbit anti-mouse Ig serum, and positive wells are selected by their ability to bind hapten-reactive partner conjugates comprising the met-sensitive moiety or NNRTI Derivative of interest. Positives from these primary screens were transferred to 24-well plates, allowed to grow for several days, then were screened by a competition reverse ELISA, wherein the hapten-reactive partner conjugates must compete with free drug i.e., lopinavir, for antibody binding sites. If the activity from the non-isotopic signal generating moiety measured when free drug was present was less than that seen when only hapten-reactive partner conjugates is present, then the antibody preferentially binds the free drug over the hapten-reactive partner conjugates form. Antibodies from these wells were cloned by serial dilution, with cloning plates screened by reverse ELISA.

The secondary screening procedure is used for both polyclonal and monoclonal antibody testing which involved taking selected antibodies and further testing them on a Cobas Bio Analyzer for inhibition of hapten-reactive partner conjugates, dose-response and cross-reactivity with various free drug solutions in the homogeneous enzyme immunoassay configuration. In the case of monoclonal antibodies, wells that produced a positive response in the assay comprising the non-isotopic signal generating moiety plus a negative response when tested in the presence of anti-HIV therapeutic were selected for further testing. The secondary screening method involves testing the degree of antibody inhibition of hapten-reactive partner conjugate, parent drug binding and cross-reactivity properties in a homogeneous assay format which simulates an assay protocol that may be used in the final kitted product. For example, instrument parameters, reagent preparation, and nonlinear data handling analysis is used. If adequate inhibition is obtained the antibody modulation property is measured in the presence of varying concentrations of anti-HIV therapeutic. Anti-HIV therapeutic standards and controls are prepared by adding known amounts of anti-HIV therapeutic to a buffered synthetic matrix. Cross-reactivity testing is performed by adding known amounts of cross reactant into human serum. The instrument used for this evaluation is the Roche Cobas Mira Chemistry Analyzer. A homogeneous enzyme immunoassay technique which can be used for the analysis is based on competition between a drug in the sample and drug labeled with the enzyme glucose-6-phosphate dehydrogenase (G6PDH) for receptor binding sites. Enzyme activity decreases upon binding to the antibody, so the drug concentration in the sample can be measured in terms of enzyme activity. Active enzyme converts nicotinamide adenine dinucleotide (NAD) to NADH, resulting in an absorbance change that is measured spectrophotometrically. Endogenous serum G6PDH does not interfere because the coenzyme functions only with the bacterial (Leuconostoc mesenteroides) enzyme employed in the assay. The quantitative analysis of drugs can be performed using human urine, serum, plasma, whole blood, or ultra filtrate.

V. C. Other Receptors

Receptors can comprise the antigen-binding domains or amino acids critical for antigen binding, e.g. antigen-binding residues, of an antibody that specifically binds the Met-Sensitive Moieties or NNRTI Derivatives. Such antigen-binding domains or residues can comprise the Complementarity-Determining Region (CDR) of an antibody. The receptors can also structurally mimic the structure represented by the antigen-binding domains or residues of a CDR. For example, if there are four amino acids within the CDR of an antibody that are critical for binding the antigen to the antibody, e.g. antigen-binding residues, then a receptor of the invention need only possess those four critical amino acids structurally arranged so as to substantially mimic their structural arrangement within the CDR of the antibody. The linkages between the critical amino acids are only important to the extent that they structurally mimic the CDR of the antibody. In this example, substitution of isosteres of the critical amino acids, such as aspartic acid for glutamic acid, are allowed.

Once the specific receptors against the met-sensitive moiety or NNRTI Derivative are available, the following immunoassay methods can be employed.

VI. Immunoassays

VI. A. Introduction

In the TDM field there are several categories of methods available for determining the presence or the concentration of met-sensitive moieties and NNRTI Derivatives in a sample. One such category is immunoassays, which are currently used to determine the presence or concentration of various analytes in biological samples, both conveniently and reliably (The Immunoassay Handbook, edited by David Wild, M Stockton Press, 1994). Generally speaking, immunoassays utilize specific receptors to target analytes in fluids, where at least one such receptor is generally labeled with one of a variety of non-isotopic signal-generating moieties.

Immunoassays usually are classified in one of several ways. One method is according to the mode of detection used, i.e., enzyme immunoassays, radio immunoassays, fluorescence polarization immunoassays, chemiluminescence immunoassays, turbidimetric assays, etc. Another grouping method is according to the assay procedure used, i.e., competitive assay formats, sandwich-type assay formats as well as assays based on precipitation or agglutination principles. In the instant application, a further distinction is made depending on whether washing steps are included in the procedure (so-called heterogeneous assays) or whether reaction and detection are performed without a washing step (so-called homogeneous assays). All the essential terms, procedures and devices are known to the skilled artisan from text books in the field, e.g., “Manual of Immunological Methods”, eds. P. Brousseau and M. Beaudet, CRC Press, 1998, and “Practice and Theory of Enzyme Immunoassays”, eds. P. Tijssen and R. H. Burdon, Elsevier Health Sciences, 1985, are herewith included by reference.

VI. B. Homogeneous and Heterogeneous Immunoassays

As mentioned above, immunoassays may be heterogeneous or homogeneous. Heterogeneous immunoassays have been applied to both small and large molecular weight analytes and require separation of bound materials (to be detected or determined) from free materials (which may interfere with that determination). Heterogeneous immunoassays may comprise a receptor or an antigen immobilized on solid surfaces such as plastic microtiter plates, beads, tubes, or the like or on membrane sheets, chips and pieces of glass, nylon, cellulose or the like (“Immobilized Enzymes, Antigens, Antibodies, and Peptides”, ed. Howard H. Weetall, Marcel Dekker, Inc., 1975). In heterogeneous immunoassays, antigen-receptor complexes bound to the solid phase are separated from unreacted and non-specific analyte in solution, generally by centrifugation, filtration, precipitation, magnetic separation or aspiration of fluids from solid phases, followed by repeated washing of the solid phase bound antigen-receptor complex.

Homogeneous assays are, in general, liquid phase procedures that do not utilize antigens or receptors that are immobilized on solid materials. Separation and washing steps are not required. In an exemplary embodiment, the antigens or receptors comprise a fluorophore signal-generating moiety, which upon binding of the antigen or receptor with a target analyte undergoes an excitation or quenching of fluorescence emissions, due to the close steric proximity of the binding pair. In another exemplary embodiment, the antigens or receptors comprise an enzyme signal-generating moiety, which upon binding of the antigen or receptor with a target analyte undergoes an enhancement or a reduction in enzyme product formation, due to a conformational change which occurs in the enzyme upon analyte binding. Homogeneous methods have typically been developed for the detection of haptens and small molecules, such as drugs, hormones and peptides.

VI. C Non-Isotopic Signal-Generating Moieties used in Immunoassays

In the methods and compositions of this application, a variety of signal-generating moieties can be employed. Among these moieties are fluorophores and enzymes. The fluorophores and enzymes discussed herein can be attached to the haptens comprising the met-sensitive moieties or NNRTI Derivatives according to the methods described elsewhere in this document.

VI. C i) Fluorophores

For the purposes of the invention a fluorophore can be a substance which itself fluoresces, can be made to fluoresce, or can be a fluorescent analogue of an analyte.

In principle, any fluorophore can be used in the assays of this invention. Preferred fluorophores, however, have the following characteristics:
a. A fluorescence lifetime of greater than about 15 nsec;
b. An excitation wavelength of greater than about 350 nm;
c. A Stokes shift (a shift to lower wave-length of the emission relative to absorption) of greater than about 20 nm;
d. For homogeneous assays, fluorescence lifetime should vary with binding status; and
e. The absorptivity and quantum yield of the fluorophore should be high.

The longer lifetime is advantageous because it is easier to measure and more easily distinguishable from the Raleigh scattering (background). Excitation wavelengths greater than 350 nm reduce background interference because most fluorescent substances responsible for background fluorescence in biological samples are excited below 350 nm. A greater Stokes shift also allows for less background interference.

The fluorophore should have a functional group available for conjugation either directly or indirectly to the Met-Sensitive antigen, NNRTI Derivative antigen, or receptor. An additional criterion in selecting the fluorophore is the stability of the fluorophore: it should not be photophysically unstable, and it should be relatively insensitive to the assay conditions, e.g., pH, polarity, temperature and ionic strength.

Preferably (though not necessarily), fluorophores for use in heterogenous assays are relatively insensitive to binding status. In contrast, fluorophores for use in homogeneous assay must be sensitive to binding status, i.e., the fluorescence lifetime must be alterable by binding so that bound and free forms can be distinguished.

Examples of fluorophores useful in the invention are naphthalene derivatives (e.g. dansyl chloride), anthracene derivatives (e.g. N-hydroxysuccinimide ester of anthracene propionate), pyrene derivatives (e.g. N-hydroxysuccinimide ester of pyrene butyrate), fluorescein derivatives (e.g. fluorescein isothiocyanate), rhodamine derivatives (e.g. rhodamine isothiocyanate), phycoerythin, and Texas Red.

VI. C ii) Enzymes

In an exemplary embodiment, the signal-generating moiety is an enzyme. From the standpoint of operability, a very wide variety of enzymes can be used. But, as a practical matter, some enzymes have characteristics which make them preferred over others. The enzyme should be stable when stored for a period of at least three months, and preferably at least six months at temperatures which are convenient to store in the laboratory, normally −20° C. or above. The enzyme should also have a satisfactory turnover rate at or near the pH optimum for binding to the receptor, this is normally at about pH 6-10, usually 6.0 to 8.0. A product should be either formed or destroyed as a result of the enzyme reaction which absorbs light in the ultraviolet region or the visible region, that is the range of about 250-750 nm., preferably 300-600 nm. The enzyme also should have a substrate (including cofactors) which has a molecular weight in excess of 300, preferably in excess of 500, there being no upper limit. The enzyme which is employed or other enzymes, with like activity, will not be present in the sample to be measured, or can be easily removed or deactivated prior to the addition of the assay reagents. Also, there should not be naturally occurring inhibitors for the enzyme present in fluids to be assayed.

Also, although enzymes of up to 600,000 molecular weight can be employed, usually relatively low molecular weight enzymes will be employed of from 10,000 to 300,000 molecular weight, more usually from about 10,000 to 150,000 molecular weight, and frequently from 10,000 to 100,000 molecular weight. Where an enzyme has a plurality of subunits the molecular weight limitations refer to the enzyme and not to the subunits.

For synthetic convenience, it is preferable that there be a reasonable number of groups to which the met-sensitive antigen, NNRTI Derivative antigen, or receptor may be bonded, particularly amino groups. However, other groups to which the met-sensitive antigen, NNRTI Derivative antigen or antibody may be bonded include hydroxyl groups, thiols, and activated aromatic rings, e.g., phenolic.

Finally, for the purposes of this invention, the enzymes should be capable of specific labeling so as to be useful in the subject assays. Specific labeling means attachment at a site related to the active site of the enzyme, so that upon binding of the receptor (met-sensitive antigen, NNRTI Derivative antigen or receptor, depending on the specific immunoassay) to the ligand (again, either the met-sensitive antigen, NNRTI Derivative antigen, or receptors), the enzyme is satisfactorily enhanced or inhibited.

Based on these criteria, the following enzymes can be used in the invention: alkaline phosphatase, horseradish peroxidase, lysozyme, glucose-6-phosphate dehydrogenase, lactate dehydrogenase, β-galactosidase, and urease. Also, a genetically engineered fragment of an enzyme may be used, such as the donor and acceptor fragment of β-galactosidase utilized in CEDIA immunoassays (see Henderson D R et al. Clin Chem. 32(9):1637-1641 (1986)); U.S. Pat. No. 4,708,929. These and other enzymes which can be used have been discussed in detail by Eva Engvall in Enzyme Immunoassay ELISA and EMIT in Methods in Enzymology, 70:419-439 (1980) and in U.S. Pat. No. 4,857,453.

In an exemplary embodiment, the enzyme is glucose-6-phosphate dehydrogenase (G6PDH) and it is attached to a hapten comprising a met-sensitive moiety or an NNRTI derivative, thus forming a hapten-reactive partner conjugate. In order to select the receptor (such as polyclonal antibodies or monoclonal antibodies) which would best interact in a homogeneous enzyme immunoassay with the hapten comprising a met-sensitive moiety or an NNRTI derivative, a variety of interrelated factors must be considered. First, the receptor must recognize and affect the activity of the hapten-reactive partner conjugate. Second, in the case of met-sensitive immunoassays, the receptor must be able to differentiate between both metabolized and unmetabolized versions of anti-HIV therapeutic. As several anti-HIV therapetics are often employed in combination, the receptor should also be selective for one anti-HIV therapeutic over the others.

The selection procedure will be demonstrated using a hapten-reactive partner conjugate comprising G6PDH as the reactive partner and a met-sensitive moiety of lopinavir as the hapten. The first step in selecting a receptor involves testing the magnitude of receptor inhibition of an hapten-reactive partner conjugate. In this step, the goal is to determine and select for those receptors which significantly inhibit the enzyme activity of G6PDH. Example 46 presents an illustration of this methodology. Receptors which perform well in the first test are then subjected to a second test. Here, the receptor is first incubated with lopinavir. Next the hapten-reactive partner conjugate is added. An exemplary receptor would preferentially bind to lopinavir instead of the hapten-reactive partner conjugate. The reduction in binding to the hapten-reactive partner conjugate would be visible as an increase G6PDH activity. Example 47 presents an illustration of this methodology.

VI. D. Detection

VI. D. i) Via Fluorescence

When a fluorescently labeled analyte (either a met-sensitive antigen, NNRTI Derivative antigen, or receptor) is employed, the fluorescence emitted is proportional (either directly or inversely) to the amount of analyte. The amount of fluorescence is determined by the amplitude of the fluorescence decay curve for the fluorescent species. This amplitude parameter is directly proportional to the amount of fluorescent species and accordingly to the analyte.

In general spectroscopic measurement of fluorescence is accomplished by:
a. exciting the fluorophore with a pulse of light;
b. detecting and storing an image of the excitation pulse and an image of all the fluorescence (the fluorescent transient) induced by the excitation pulse;
c. digitizing the image;
d. calculating the true fluorescent transient from the digitized data;
e. determining the amplitude of the fluorescent transient as an indication of the amount of fluorescent species.

According to the method, substantially all of the fluorescence emitted by the fluorescent species reaching the detector as a function of time from the instant of excitation is measured. As a consequence, the signal being detected is a superimposition of several component signals (for example, background and one analyte specific signal). As mentioned, the individual contributions to the overall fluorescence reaching the detector are distinguished based on the different fluorescence decay rates (lifetimes) of signal components. In order to quantitate the magnitude of each contribution, the detected signal data is processed to obtain the amplitude of each component. The amplitude of each component signal is proportional to the concentration of the fluorescent species.

VI. D. ii) Via Enzyme

Detection of the amount of product produced by the hapten-reactive partner conjugate of the invention can be accomplished by several methods which are known to those of skill in the art. Among these methods are colorimetry, fluorescence, and spectrophotometry. These methods of detection are discussed in “Analytical Biochemistry” by David Holme, Addison-Wesley, 1998, which is incorporated herein by reference.

VI. E. Lateral Flow Chromatography

The compounds and methods of the invention also encompass the use of these materials in lateral flow chromatography technologies. The essence of lateral flow chromatography involves a membrane strip which comprises a detection device, such as a non-isotopic signal generating moiety, for the anti-HIV therapeutic of interest. A sample from a patient is then applied to the membrane strip. The sample interacts with the detection device, producing a result. The results can signify several things, including the absence of the anti-HIV therapeutic in the sample, the presence of the anti-HIV therapeutic in the sample, and even the concentration of the anti-HIV therapeutic in the sample.

In one embodiment, the invention provides a method of qualitatively determining the presence or absence of an anti-HIV therapeutic in a sample, through the use of lateral flow chromatography. The basic design of the qualitative lateral flow device is as follows: 1) The sample pad is where the sample is applied. The sample pad is treated with chemicals such as buffers or salts, which, when redissolved, optimize the chemistry of the sample for reaction with the conjugate, test, and control reagents. 2) Conjugate release pad is typically a polyester or glass fiber material that is treated with a conjugate reagent such as an antibody colloidal gold conjugate. A typical process for treating a conjugate pad is to use impregnation followed by drying. In use, the liquid sample added to the test will redissolve the conjugate so that it will flow into the membrane. 3) The membrane substrate is usually made of nitrocellulose or a similar material whereby antibody capture components are immobilized. 4) A wicking pad is used in tests where blood plasma must be separated from whole blood. An impregnation process is usually used to treat this pad with reagents intended to condition the sample and promote cell separation. 5) The absorbent pad acts as a reservoir for collecting fluids that have flowed through the device. 6) The above layers and membrane system are laminated onto a plastic backing with adhesive material which serves as a structural member.

In another embodiment, the invention provides a method of qualitatively determining the presence of an anti-HIV therapeutic in a sample, through the use of lateral flow chromatography. In this embodiment, the membrane strip comprises a sample pad, which is a conjugate release pad (CRP) which comprises a receptor that is specific for the anti-HIV therapeutic of interest. This receptor is conjugated to a non-isotopic signal-generating moiety, such as a colloidal gold particle. Other detection moieties useful in a lateral flow chromatography environment include dyes, colored latex particles, fluorescently labeled latex particles, non-isotopic signal generating moieties, etc. The membrane strip further comprises a capture line, in which the met-sensitive moiety or NNRTI Derivative is immobilized on the strip. In some embodiments, this immobilization is through covalent attachment to the membrane strip, optionally through a linker. In other embodiments, the immobilization is through non-covalent attachment to the membrane strip. In still other embodiments, the immobile met-sensitive moiety or NNRTI Derivative in the capture line is attached to a reactive partner, such as an immunogenic carrier like BSA.

Sample from a patient is applied to the sample pad, where it can combine with the receptor in the CRP, thus forming a solution. This solution is then allowed to migrate chromatographically by capillary action across the membrane. When the anti-HIV therapeutic of interest is present in the sample, an anti-HIV therapeutic-receptor complex is formed, which migrates across the membrane by capillary action. When the solution reaches the capture line, the anti-HIV therapeutic-receptor complex will compete with the immobile anti-HIV therapeutic for the limited binding sites of the receptor. When a sufficient concentration of anti-HIV therapeutic is present in the sample, it will fill the limited receptor binding sites. This will prevent the formation of a colored receptor-immobile anti-HIV therapeutic complex in the capture line. Therefore, absence of color in the capture line indicates the presence of anti-HIV therapeutic in the sample.

In the absence of anti-HIV therapeutic in the sample, a colored receptor-immobile anti-HIV therapeutic complex will form once the solution reaches the capture line of the membrane strip. The formation of this complex in the capture line is evidence of the absence of anti-HIV therapeutic in the sample.

In another embodiment, the invention provides a method of quantitatively determining the amount of an anti-HIV therapeutic in a sample, through the use of lateral flow chromatography. This technology is further described in U.S. Pat. Nos. 4,391,904; 4,435,504; 4,959,324; 5,264,180; 5,340,539; and 5,416,000, among others, which are herein incorporated by reference. In one embodiment, the receptor is immobilized along the entire length of the membrane strip. In general, if the membrane strip is made from paper, the receptor is covalently bound to the membrane strip. If the membrane strip is made from nitrocellulose, then the receptor can be non-covalently attached to the membrane strip through, for example, hydrophobic and electrostatic interactions.

The membrane strip comprises a CRP which comprises the anti-HIV therapeutic of interest attached to a detector moiety. In an exemplary embodiment, the detector moiety is an enzyme, such as horseradish peroxidase (HRP).

Sample from a patient is applied to the membrane strip, where it can combine with the anti-HIV/detector molecule in the CRP, thus forming a solution. This solution is then allowed to migrate chromatographically by capillary action across the membrane. When the anti-HIV therapeutic of interest is present in the sample, both the sample anti-HIV therapeutic and the anti-HIV/detector molecule compete for the limited binding sites of the receptor. When a sufficient concentration of anti-HIV therapeutic is present in the sample, it will fill the limited receptor binding sites. This will force the anti-HIV/detector molecule to continue to migrate in the membrane strip. The shorter the distance of migration of the anti-HIV/detector molecule in the membrane strip, the lower the concentration of anti-HIV therapeutic in the sample, and vice versa. When the anti-HIV/detector molecule comprises an enzyme, the length of migration of the anti-HIV/detector molecule can be detected by applying an enzyme substrate to the membrane strip. Detection of the product of the enzyme reaction is then utilized to determine the concentration of the anti-HIV therapeutic in the sample. In another exemplary embodiment, the enzyme's color producing substrate such as a modified N,N-dimethylaniline is immobilized to the membrane strip and 3-methyl-2-benzothiazolinone hydrazone is passively applied to the membrane, thus alleviating the need for a separate reagent to visualize the color producing reaction.

VII. Kits

Another aspect of the present invention relates to kits useful for conveniently determining the presence or the concentration of active anti-HIV therapeutic in a sample. The invention also encompasses kits useful for conveniently determining the presence or the concentration of a NNRTI, both active and inactive, in a sample. The kits of the present invention can comprise a receptor specific for a met-sensitive moiety of an anti-HIV therapeutic or a NNRTI. In an exemplary embodiment, the receptor is an antibody. In another exemplary embodiment, the receptor comprises the antigen-binding domain or antigen-binding residues that specifically bind to the met-sensitive moiety of an anti-HIV therapeutic or a NNRTI Derivative. The kits can optionally further comprise calibration and control standards useful in performing the assay; and instructions on the use of the kit. The kits can also optionally comprise a hapten-reactive partner conjugate. To enhance kit versatility, the kit components can be in a liquid reagent form, a lyophilized form, or attached to a solid support. The reagents may each be in separate containers, or various reagents can be combined in one or more containers depending on cross-reactivity and stability of the reagents.

Any sample that is reasonably suspected of containing the analyte, i.e., a met-sensitive moiety of a PI or NNRTI, or a NNRTI, can be analyzed by the kits of the present invention. The sample is typically an aqueous solution such as a body fluid from a host, for example, urine, whole blood, plasma, serum, saliva, semen, stool, sputum, cerebral spinal fluid, tears, mucus, breast milk or the like. In an exemplary embodiment, the sample is plasma or serum. The sample can be pretreated if desired and can be prepared in any convenient medium that does not interfere with the assay. For example, the sample can be provided in a buffered synthetic matrix.

The sample, suspected of containing anti-HIV therapeutic, and a calibration material, containing a known concentration of the anti-HIV therapeutic, are assayed under similar conditions. Anti-HIV therapeutic concentration is then calculated by comparing the results obtained for the unknown specimen with results obtained for the standard. This is commonly done by constructing a calibration or dose response curve.

Various ancillary materials will frequently be employed in an assay in accordance with the present invention. In an exemplary embodiment, buffers and/or stabilizers are present in the kit components. In another exemplary embodiment, the kits comprise indicator solutions or indicator “dipsticks”, blotters, culture media, cuvettes, and the like. In yet another exemplary embodiment, the kits comprise indicator cartridges (where a kit component is bound to a solid support) for use in an automated detector. In still another exemplary embodiment, additional proteins, such as albumin, or surfactants, particularly non-ionic surfactants, may be included. In another exemplary embodiment, the kits comprise an instruction manual that teaches a method of the invention and/or describes the use of the components of the kit.

### EXAMPLES

The following examples are offered by way of illustration and not by way of limitation. Chemicals were purchased from Aldrich Chemical Co. (Milwaukee, Wis.), and used as received. Amino acids derivatives and resins were purchased from AnaSpect (San Jose, Calif.) or Advanced Chem Tech (ACT) (Louisville, Ky.). Silica gel plates were obtained from Analtech (Newark, Del.). NMR spectra were recorded on a 300 MHz Brucker instrument. Chemical shifts are in ppm downfield from TMS and were recorded in the solvents listed. Splitting patterns are designated as follows: s, singlet; d, doublet; t, triplet; m, multiplet; br, broad. The chemical synthesis and characterization of compounds carried out by Kimia Corp. (Santa Clara, Calif.).

### Example 1

Preparation of a Hapten Comprising Met-Sensitive Moiety (A1)

1.1 Preparation of S(+)-3-hydroxytetrahydrofuran carbomyl-N-phenylalanine 2

A solution of Fmoc phenylalanine (3.3 g, 8.64 mmol) and DIEA (3.0 mL, 17.28 mmol) in dried dicholoromethane (DCM) (10 mL) was added to the chlorotrityl resin (1.08 mmol/g, 2 g). The suspension was shaken overnight at rt. The resin was then washed with DMF (3×10 mL), DCM (3×10 mL) and MeOH (3×10 mL) respectively and dried in vacuo to give 3.1 g of the resin. The resin gave a negative test for ninhydrin. To the resin was added a solution of 20% piperidine in DMF (15 mL) and the mixture was shaken for 30 min on a shaker. The resin was then filtered and washed with DMF (3×20 mL), DCM (3×20 mL) and MeOH (2×20 mL) respectively. The resin gave a positive test for ninhydrin. The choloroformate 5 (prepared by reaction of the alcohol with excess phosgene) was then added slowly to suspension of the resin in DCM (5 mL) and DIEA (1.9 mL, 11.9 mmol) at rt and the suspension was shaken for 2 h. After this time a sample of the resin was shown to be negative for ninhydrin test. The resin was filtered and washed with a solution of 10% DIEA in DCM (10 mL), DCM (3×15 mL) and MeOH (15 mL) respectively. The resin was then dried in vacuo to dryness. To the resin was then added a mixture of TFA, AcOH and DCM (10 mL, 1:1:8) and shaken for 30 min. The resin was filtered and washed with DCM (10 mL). The combined filtrates were evaporated to dryness in vacuo to give 617 mg of the crude product as a viscous oil. The crude product was then dissolved in EtOAc (10 mL) and treated with a saturated solution of bicarbonate (3 mL). The pH of the aqueous layer was 12. Water (10 mL) was then added and the aqueous layer was separated. The aqueous layer was extracted with EtOAc (2×20 mL). The aqueous layer was then acidified by slow addition of HCl (1N) to pH 4. The acidic solution was then extracted with EtOAc (2×20 mL). The organic layer was then washed with brine (5 mL) and dried (Na₂SO₄). The solvent was then removed in vacuo to give the pure product 2 (399 mg, 1.43 mmol, 16.5%) as a white solid.

1.2 Characterization of Product

¹H NMR (CDCl₃): 7.28 (m, 3H); 7.18 (m, 2H); 5.08 (s, 1H); 5.22 (m, 1H); 5.75 (d, 1H); 4.65 (m, 1H); 3.83 (m, 4H); 3.20 (dd, 1H); 3.09 (dd, 1H); 2.07 (m, 2H).

### Example 2

Preparation of a Hapten Comprising Met-Sensitive Moiety (A2)

2.1 Preparation of 4

A solution of t-Boc epoxide 3 (2.63 g, 10 mmol) in saturated solution of ammonia in MeOH (50 mL) at ice bath temperature was stirred for 4 h. The solvent was then removed under reduced pressure. The crude residue was dissolved in THF (50 mL), DIEA (1.89 mL, 11 mmol) and benzylcholoro formate (1.87 g, 11 mmol) and stirred overnight. The reaction was quenched with water (50 mL) and extracted with ethyl acetate (2×100 mL). The combined organic layers were washed with saturated Na₂CO₃ (100 mL), brine (100 mL), dried (Na₂SO₄) and evaporated to dryness. The crude residue was purified on a column (silica gel, ethyl acetate:hexane, 60:40) to give the cbz protected product (2.48 g, 60%) as a foam. The product was dissolved in THF/HCl (4N, 100 mL) and stirred for 2 h. The solvent was removed to give pure 6 as a white solid (1.88 g, 100%).

2.2 Preparation of 6

To a stirred solution of amine 4 (942 mg, 3 mmol) and DIEA (1 mL) in THF (10 mL) was added choloroformate 1 (as described before, 461 mg, 3.1 mmol) at ice bath tempreture over a period of 1 hour. The reaction was then allowed to warm to RT overnight. To the reaction mixture was then added water (50 mL) and the mixture was extracted with DCM (3×30 mL). The combined organic layers were washed with saturated Na₂CO₃ (10 mL), brine (30 mL) and dried (Na₂SO₄). The solvent was removed under reduced pressure and the residue was purified on a column (silica gel, DCM:MeOH, 95:5) to give (1.03 g, 80%) of the cbz protected product that was hydrogenated as described before to give 3-amino-1-benzyl-2-hydroxy-propyl)-carbamic acid tetrahydro-furan-3-yl ester, 6 (705 mg) as a white solid.

### Example 3

Preparation of a Hapten Comprising Met-Sensitive Moiety (A3)

3.1 Preparation of 7

To a stirred solution of the acid 2 (279 mg, 1 mmol) in DMF (2 mL) was added DCC (260 mg, 1.2 mmol) and NHS (120 mg, 1.4 mmol). The mixture was stirred for 6 h and then glycine (150 mg, 2 mmol) and DIEA (0.4 mL, 2 mmol) were added at rt and the reaction was stirred overnight. The solvent was then evaporated to dryness in vacuo. To the residue was added water (10 mL) and extracted with ethyl acetate (2×25 mL). The combined organic layer was then washed with HCl (1N, 4 mL), and saturated sodium bicarbonate (3 mL) and dried (Na₂SO₄). The ethyl acetate was removed under reduced pressure to give the crude product. The crude product was purified on a silica gel column (MeOH:DCM:AcOH, 10:90:0.1) to give pure product 7 (221 mg, 66%) as a white solid.

### Example 4

Preparation of a Hapten Comprising Met-Sensitive Moiety (A4)

4.1 Preparation of Bromoacetyl Derivative of 6

To a stirred solution of the amine 6 (148 mg, 0.5 mmol) in DMF (3 mL) was added bromo acetyl NHS ester (130 mg, 0.6 mmol). The mixture stirred overnight and then diluted with water (10 mL). The mixture was then extracted with DCM (3×30 mL). The combined DCM layers were washed with brine (30 mL), dried (Na₂SO₄) and evaporated to dryness in vacuo. The crude was then purified on a column (silica gel, DCM:MeOH, 95:5) to give the bromoacetyl of 8 (124 mg, 60%) as a white solid.

### Example 5

Preparation of a Hapten Comprising Met-Sensitive Moiety (B1)

5.1 Preparation of 10

To a stirred solution of N-methylcarboxy 2-t-butyl alanine 2 (667 mg, 3.52 mmol) in DCM (10 mL) was added DCC (800 mg, 3.88 mmol) and HOBT (524 mg, 3.88 mmol). The mixture was stirred for 30 min and then phenylalanine-O-t-butyl ester (1 g, 3.88 mmol) and DIEA (2.0 mL, 11.5 mmol) were added at rt and the reaction was stirred overnight (under Ar). The solvent was then evaporated to dryness in vacuo. To the residue was added water (30 mL) and extracted with ethyl acetate (2×50 mL). The combined organic layer was then washed with HCl (1N, 20 mL), and saturated sodium bicarbonate (20 mL) and dried (Na₂SO₄). The ethyl acetate was removed under reduced pressure to give the t-BOC protected crude product. The crude product was purified on a silica gel column (EtOAc:hexane 1:3) to give the pure t-BOC protected product (720 mg) as a white solid. To this compound was added a solution of HCl in dioxane (4N, 5 mL) at rt and the reaction mixture was stirred overnight. The solvent was removed in vacuo and the residue was purified on a silica gel column (DCM:MeOH 95:5) to give the pure product 10 as a white solid (330 mg, 0.98 mmol, 27.5%).

5.2 Characterization of Product

¹H NMR (DMSO): 12.60 (s, 1H); 8.20 (s, 1H); 7.22 (m, 5H); 6.89 (d, 1H); 4.44 (m, 1H); 3.95 (d, 1H); 3.55 (s, 3H); 3.15 (dd, 1H); 2.88 (dd, 1H) and 0.96 (s, 6H).

### Example 6

Preparation of a Hapten Comprising Met-Sensitive Moiety (B2)

6.1 Preparation of 11

To a stirred solution of N-methylcarboxy 2-t-butyl alanine 2 (567 mg, 3 mmol) in DCM (10 mL) was added DCC (800 mg, 3.88 mmol) and NHS (460 mg, 4 mmol). The mixture was stirred for 6 h and then 4 (942 mg, 3 mmol) and DIEA (1.0 mL, 5.5 mmol) were added at rt and the reaction was stirred overnight. The solvent was then evaporated to dryness in vacuo. To the residue was added water (60 mL) and extracted with ethyl acetate (2×50 mL). The combined organic layer was then washed with HCl (1N, 20 mL), and saturated sodium bicarbonate (20 mL) and dried (Na₂SO₄). The ethyl acetate was removed under reduced pressure to give the crude product. The crude product was purified on a silica gel column (EtOAc:hexane, 1:3) to give pure cbz protected product (745 mg, 50%) as a white solid. The cbz protected product (745 mg) was hydrogenated with 10% Pd/C (150 mg) under atmospheric pressure in MeOH (50 mL) overnight. The reaction mixture was then passed through a pad of Celite. The filtrate was concentrated to dryness under reduced pressure to give pure 11 (646 mg).

### Example 7

Preparation of a Hapten Comprising Met-Sensitive Moiety (B3)

7.1 Preparation of 12

To a stirred solution of 10 (336 mg, 1 mmol) in DMF (2 mL) was added DCC (260 mg, 1.2 mmol) and NHS (120 mg, 1.4 mmol). The mixture was stirred for 6 h and then glycine (150 mg, 2 mmol) and DIEA (0.4 mL, 2 mmol) were added at rt and the reaction was stirred overnight. The solvent was then evaporated to dryness in vacuo. To the residue was added water (10 mL) and extracted with ethyl acetate (2×25 mL). The combined organic layers were then washed with HCl (1N, 4 mL), and saturated sodium bicarbonate (3 mL) and dried (Na₂SO₄). The ethyl acetate was removed under reduced pressure to give the crude product. The crude product was purified on a silica gel column (MeOH:DCM:AcOH, 10:90:0.1) to give pure product 12 (221 mg, 66%) as a white solid.

### Example 8

Preparation of a Hapten Comprising Met-Sensitive Moiety (B4)

8.1 Preparation of 11

To a stirred solution of 11 (161 mg, 0.5 mmol) in DMF (5 mL) was added bromo acetyl NHS ester (130 mg, 0.6 mmol). The mixture stirred overnight and then diluted with water (20 mL). The mixture was then extracted with DCM (3×20 mL). The combined DCM layers were washed with brine (20 mL), dried (Na₂SO₄) and evaporated to dryness under vacuum. The crude was then purified on a column (silica gel, DCM:MeOH, 95:5) to give the bromoacetyl 13 (177 mg, 75%) as a white foam.

### Example 9

Preparation of a Hapten Comprising Met-Sensitive Moiety (B5)

9.1 Preparation of 14

To a stirred solution of 11 (175 mg, 0.5 mmol) in DMF (1 mL) was added succincyl anhydride (60 mg, 0.6 mmol) and DIEA (80 μL). The reaction mixture was stirred overnight and then water was added (2 mL). The pH of resulting mixture was adjusted to 2 by addition of HCl (1N) and extracted with DCM (2×10 mL). The combined DCM layer was evaporated to dryness and the residue was purified on a column (silica gel, DCM:MeOH:AcOH, 95:5:0.1) to give the desired product 14 (180 mg, 80%) as yellow solid.

### Example 10

Preparation of a Hapten Comprising Met-Sensitive Moiety (C1)

10.1 Preparation of 16

To a stirred solution of the t-BOC piperazine 15 (570 mg, 2 mmol) and DIEA (363 μL, 2.1 mmol) in THF (5 mL) was added bromohydrin (408 mg, 3 mmol) and the reaction was stirred overnight. To the reaction was then added concentrated solution of ammonia (10N, 0.5 mL) and stirring was continued for further 3 h. To the reaction mixture was then added water (10 mL) and extracted with DCM (3×25 mL). The combined DCM layers were washed with brine (25 mL), dried (Na₂SO₄) and evaporated to dryness. The crude residue was dissolved in THF (5 mL), DIEA (345 μL, 2 mmol) and benzylcholoro formate (374 mg, 2.2 mmol) and stirred overnight. The reaction was quenched with water (10 mL) and extracted with ethyl acetate (2×25 mL). The combined organic layers were washed with brine (20 mL), dried (Na₂SO₄) and evaporated to dryness. The crude residue was purified on a column (silica gel, ethyl acetate:hexane, 60:40) to give 16 (590 mg, 0.60%) as a foam.

10.2 Preparation of 17

To a stirred solution of HCl (4N) in THF (10 mL) was added 16 (490 mg, 1 mmol) at RT. The mixture was stirred for 2 h. The reaction mixture was then evaporated to dryness under vacuum. To a solution of the residue and triethylamine (400 mg) in THF (10 mL) was added 3-picolyl chloride HCl salt (245 mg, 1.5 mmol) and the reaction mixture was heated to reflux over night. Water (50 mL) was added to the reaction and the milky reaction mixture was extracted with ethyl acetate (3×50 mL). The combined ethyl acetate phase was washed with brine (40 mL) and dried (Na₂SO₄). The solvent was then removed in vacuo to give the crude product as a yellow foam that was purified on a silica gel column (DCM: MeOH, 90:10) to cbz protected indinavir 17 (289 mg, 60%) as a yellow solid. To a stirred solution of the cbz protected 17 in MeOH (5 mL) was added Pd/C (10%, 50 mg) and hydrogenated at atmospheric pressure overnight. The mixture was filtered over Celite and concentrated in vacuo to give the pure 17 as a white solid (241 mg, 99%).

### Example 11

Preparation of a Hapten Comprising Met-Sensitive Moiety (C2)

11.1 Preparation of 19

To a stirred solution of 17 (175 mg, 0.5 mmol) in DMF (5 mL) was added bromo acetyl NHS ester (130 mg, 0.6 mmol). The mixture stirred overnight and then diluted with water (20 mL). The mixture was then extracted with DCM (3×20 mL). The combined DCM layers were washed with brine (20 mL), dried (Na₂SO₄) and evaporated to dryness in vacuo. The crude material was then purified on a column (silica gel, DCM:MeOH, 95:5) to give compound 19 (195 mg, 82%) as a white foam.

### Example 12

Preparation of a Hapten Comprising Met-Sensitive Moiety (C3)

12.1 Preparation of 21

A solution of product 20 (580 mg) was treated with HCl to remove the BOC protecting group. The resulting compound was reacted with 3-picolyl chloride and then hydrogenated to remove the Cbz group as described in the previous experiment. The product of synthesis was 21.

### Example 13

Preparation of a Hapten Comprising Met-Sensitive Moiety (C4)

13.1 Preparation of 22

A stirred solution of piperazine 15 (1.1 g, 3.84 mmol), the Cbz-glycidyl (996 mg, 3.84 mmol) and DIEA (550 μL) in 25 mL of DMF was heated at 65° C. for 10 h. The reaction was quenched by the addition of NaHCO₃ (3 mL, 5%). The reaction was then extracted with isopropyl acetate (2×40 mL). The organic layer was washed with brine (10 mL), dried (Na₂SO₄) and evaporated to dryness. The oily residue was purified by flash chromatography (silica gel, EtOAc:hexane, 50:50) to give the pure product (950 mg, 70%) as a yellow oil. The product (950 mg) was dissolved in MeOH (20 mL) and hydrogenated at atmospheric pressure with Pd/C (10%, 30 mg) to give the desired amine that was used for the next step without further purification. A solution of the product in DMF (10 mL) and bromo t-butyl acetate (1.2 eq) and K₂CO₃ (130 mg) was heated overnight at 65° C. To the reaction mixture was the added water (100 mL) the milky mixture was extracted with DCM (3×100 mL). The combined organic layers were washed with brine (50 mL) and dried (Na₂SO₄). The organic layer was evaporated to dryness and the oily residue was purified on a silica gel column (ethyl acetate:hexane, 50:50) to give 650 mg of the desire product. A solution of the product (600 mg) in isopropanol (5 mL) at ice bath temperature was added to a solution of HCl (6N, 2 mL). The reaction was stirred for 15 min and then concentrated HCl (1 mL) was added and the reaction kept at 0° C. for 1 h. The reaction was then warmed to rt and stirred for 4 h. The mixture was then cooled with an ice bath and the pH was adjusted to 3 by slow addition of NaOH (20%). The mixture was then extracted with ethyl acetate (3×50 mL). The combined organic layers were washed with brine (2×50 mL) and dried (Na₂SO₄) to give the HCl salt of the deprotected product (450 mg). To a solution of this product (400 mg) and triethylamine (400 mg) in THF (10 mL) was added 3-picolyl chloride HCl salt (1.5 eq) and the reaction mixture was heated to reflux overnight. Water (50 mL) was added to the reaction and the milky reaction mixture was extracted with ethyl acetate (3×50 mL). The combined ethyl acetate phase was washed with brine (40 mL) and dried (Na₂SO₄). The solvent was then removed in vacuo to give the crude product as a yellow foam that was purified on a silica gel column (DCM: MeOH, 90:10) to give 345 mg of 22 as a pale yellow solid.

### Example 14

Preparation of a Hapten Comprising Met-Sensitive Moiety (C5)

14.1 Preparation of 23

A solution of product 20 (580 mg) was treated with HCl to remove the BOC protecting group, reacted with 3-picolyl chloride and hydrogenated to remove the Cbz group as described in the previous experiment. The resulting amine was reacted with 1.4 eq of bromo acetyl NHS ester in THF to give 210 mg of the desire product 23 after purification on a column (silica gel, DCM:MeOH, 95:5) as a pale yellow foam.

### Example 15

Preparation of a Hapten Comprising Met-Sensitive Moiety (1))

15.1 Preparation of 24

To a stirred suspension of valine (5 g, 42.7 mmol), potassium hydrogen carbonate (6.4 g, 64 mmol) and water (30 mL) was added phenyl carbonochloridate (5.6 mL, 44.8 mmol). The pH was adjusted to 8.15-8.6 with 50% NaOH and kept between 8.5-8.7 through the periodic addition of 50% NaOH. When the pH stabilized at 8.6-8.7 the mixture was stirred at rt for 90 min. The pH was adjusted to 8.9 and the solution was diluted with methyl tert-butyl ether (30 mL) and filtered to remove solids. The aqueous layer was added to 30% aq. H₂SO₄ (100 mL) and extracted with methyl tert-butyl ether (50 mL). The organic layer was dried over Na₂SO₄, filtered and concentrated under reduced pressure to afford a clear viscous oil; yield: 9.05 g (90%).

A solution of the above oil (9.05 g, 38.4 mmol) in THF (100 mL) and 3-chloropropylamine hydrochloride (4.8 g, 36.9 mmol) was cooled to 2° C. Solid NaOH (4.6 g, 115 mmol) was added to the stirring suspension. The reaction was stirred at less than 10° C. until the valine derivative was completely consumed, then stirred at rt for 16 h. Water (70 mL) was added and extracted with EtOAc (2×30 mL). The aqueous layer was acidified to pH=3.4 and extracted with EtOAc (2×50 mL), dried over Na₂SO₄, filtered and concentrated to give 8.8 g of mixture of acids. This acid was dissolved in THF (150 mL) and added dropwise to a suspension of 60% NaH in oil (6 g, 150 mmol) in dry THF (100 mL) at 0° C. The mixture was stirred for overnight and treated with ice water (100 mL). The organic layer was separated and the aqueous layer was acidified to pH=1 and extracted with chloroform (4×70 mL). The organic layer was dried over Na₂SO₄, filtered and concentrated to give 4.5 g of a white solid. This solid was dissolved in hot CHCl₃ (150 mL), EtOAc (30 mL) was then added and allowed to cool to rt. The solid was filtered and dried in vacuum to give 2.13 g of 24.

15.2 Characterization Data for 24

¹HNMR (DMSO-d₆): 6(12.5, s, 1H), 6.3 (s, 1H), 4.4 (1H, d, J=10.5 Hz), 3.1-3.2 (m, 4H) 2.0 (m, 1H), 1.7 (m, 2H), 0.92 (d, 3H, J=6.6 Hz), 0.81 (d, 3H, J=6.6 Hz); Mass: 201 (m+1).

### Example 16

Preparation of a Hapten Comprising Met-Sensitive Moiety (D2)

16.1 Preparation of 25

To a solution of 24 (1.0 g, 5 mmol) in DCM/DMF (25 mL/2 mL) was added DCC (1.13 g, 5.5 mmol), HOBT (0.74 g, 5.5 mmol) and phenylalanine t-butyl ester (1.21 g, 5.5 mmol) and DIEA (2.6 mL, 15 mmol). The mixture was stirred overnight, filtered and washed with 2.5% NaOH, 1N HCl, water, brine, dried over Na₂SO₄, filtered and concentrated to give 1.79 g of crude ester of 25. The crude was purified on a silica gel column using EtOAc:hexanes (10:1 to 1:1) to give a mixture of two compounds. The yield was 1.04 g. 400 mg of this mixture was treated with TFA (3 mL) and stirred overnight. The TFA was then removed under reduced pressure. The crude product was purified on a silica gel column using DCM:MeOH:AcOH (95:5:0.3) to give 200 mg of 2-[3-methyl-2-(2-oxo-tetrahydro-pyrimidin-1-yl)-butyrylamino]-3-phenyl-propionic acid, 25.

16.2 Characterization Data for 25

¹H NMR (CDCl₃): δ=7.13-7.25 (m, 5H), 6.96 (s, 1H), 6.47 (s, 1H), 5.96 (s, 1H), 4.85 (dd, 1H, J=4.8 Hz, 8.4 Hz), 4.82 (dd, 1H, J=4.8 Hz, 8.1 Hz), 4.24-3.80 (d, 1H, J=11.4 Hz), 2.87-3.40 (m, 6H), 1.6-2.6 (m, 3H), 1.4-0.90 (d, 3H, J=6.3 Hz), 0.97-0.83 (d, 3H, J=6.6 Hz).

### Example 17

Preparation of a Hapten Comprising Met-Sensitive Moiety (D3)

17.1 Preparation of 26

To a solution of N-BOC phenylalanal (5 mmol) in MeOH (10 mL) was added NH₄OAc (15 mmol) and NaBH₃CN (6 mmol). The mixture was then stirred overnight. MeOH was evaporated under reduced pressure and the residue was partitioned between water and EtOAc. The organic layer was washed with water and brine, dried over Na₂SO₄ and concentrated. The residue was dissolved in DCM and treated with FmocOSU (5 mmol). The solution was stirred for 4 h. The mixture was washed with water, dried, concentrated and purified by column chromatography. The resulting compound was treated with 20% TFA in DCM for overnight then concentrated in vacuo to give the TFA salt of 2-amino-3-phenyl-propyl)-carbamic acid 9H-fluoren-9-ylmethyl ester.

To a solution of 24 in DCM was added DCC (1.1 eq), HOBT (1.1 eq), (2-amino-3-phenyl-propyl)-carbamic acid 9H-fluoren-9-ylmethyl ester, TFA salt (1.1 eq), DIEA (2 eq). The mixture was stirred overnight and then filtered. The filtrate was washed with 1N NaOH, 1N HCl, water and brine, dried over Na₂SO₄ and concentrated. The residue was dissolved in DCM and treated with 20% piperidine in DCM for 1 h. The solvent was then removed under a reduced pressure and the residue was purified using a silica gel column to give N-(2-amino-1-benzyl-ethyl)-3-methyl-2-(2-oxo-tetrahydro-pyrimidin-1-yl)-butyramide, 26.

### Example 18

Preparation of a Hapten Comprising Met-Sensitive Moiety (D4)

18.1 Preparation of 27

To a stirred solution of the acid (600 mg, 3 mmol) in DCM (20 mL) was added DCC (800 mg, 3.88 mmol) and NHS (460 mg, 4 mmol). The mixture was stirred for 6 h and then 4 (942 mg, 3 mmol) and DIEA (1.0 mL, 5.5 mmol) were added and gradually warmed to rt and the reaction was stirred overnight. The solvent was then evaporated to dryness in vacuo. To the residue was added water (80 mL) and extracted with ethyl acetate (2×50 mL). The combined organic layer was then washed with HCl (1N, 20 mL), and saturated sodium bicarbonate (30 mL) and dried (Na₂SO₄). The ethyl acetate was removed under reduced pressure to give the crude product. The crude product was purified on a silica gel column (EtOAc:hexane, 1:3) to give pure cbz protected product (257 mg, 50%) as a yellow solid. The cbz protected product (515 mg) was hydrogenated with 10% Pd/C (150 mg) under atmospheric pressure in MeOH (50 mL) overnight. The reaction mixture was then passed through a pad of Celite. The filterate was concentrated to dryness under reduced pressure to give pure deprotected amine (190 mg). To a stirred solution of the amine (181 mg, 0.5 mmol) in DMF (7 mL) was added bromo acetyl NHS ester (130 mg, 0.6 mmol). The mixture stirred overnight and then diluted with water (20 mL). The mixture was then extracted with DCM (3×30 mL). The combined DCM layers were washed with brine (30 mL), dried (Na₂SO₄) and evaporated to dryness in vacuo. The crude was then purified on a column (silica gel, DCM:MeOH, 95:5) to give the bromoacetyl 27 (193 mg, 75%) as a white foam.

### Example 19

Preparation of a Hapten Comprising Met-Sensitive Moiety (E1)

19.1 Preparation of 29

To a stirred solution of 3-hydroxy-2-methyl-benzoic acid 28 (1.52 g, 10 mmol) in THF (5 mL) was added HBTU (3.8 g, 1 mmol) at −10° C. The mixture was stirred for 3 h and then a suspension of S-phenyl cysteine (3.94 g, 20 mmol) and DIEA (1 mL, 6 mmol) in DMF (5 mL) was added. The mixture was then stirred overnight. Water (20 mL) was added and the mixture was extracted with ethyl acetate (4×50 mL). The combined organic layers were reduced to 50 mL in vacuo and extracted with saturated solution of NaHCO₃ (20 mL). The aqueous layer was acidified with HCl (1N) to pH 3 and then was extracted with EtOAc (3×30 mL). The combined organic layer was dried (Na₂SO₄) and concentrated to dryness to give crude compound 29 (840 mg, 2.51 mmol, 25%) as a thick liquid. The crude was further purified on silica gel (CH₂Cl₂:MeOH:AcOH: 90:10:0.2) to give pure 29 (114 mg, 0.34 mmol, 3.4%) as a tan solid.

19.2 Characterization Data for 29

¹H NMR (DMSO): 9.47 (s, 1H); 8.44 (d, 1H); 7.37 (m, 4H); 7.20 (m, 1H); 7.00 (t, 1H); 6.82 (d, 1H); 6.71 (d, 1H); 4.42 (m, 1H); 3.46 (dd, 1H); 3.22 (dd, 1H); 2.12 (s, 3H).

### Example 20

Preparation of a Hapten Comprising Met-Sensitive Moiety (E2)

20.1 Preparation of 30

To a stirred solution of acid 29 (331 mg, 1 mmol) in THF (2 mL) was added DCC (260 mg, 1.2 mmol) and NHS (120 mg, 1.4 mmol) at −10° C. The mixture was stirred for 6 h and then glycine (150 mg, 2 mmol) and DIEA (0.4 mL, 2 mmol) were added and the reaction was allowed to warm to rt and the reaction was stirred overnight. The solvent was then evaporated to dryness in vacuo. To the residue was added water (10 mL) and extracted with ethyl acetate (2×25 mL). The combined organic layer was then washed with HCl (1N, 4 mL), and saturated sodium bicarbonate (3 mL) and dried (Na₂SO₄). The ethyl acetate was removed under reduced pressure to give the crude product. The crude product was purified on a silica gel column (MeOH:DCM:AcOH, 10:90:0.1) to give pure product 30 (221 mg, 66%) as a white solid.

### Example 21

Preparation of a Hapten Comprising Met-Sensitive Moiety (E3)

21.1 Preparation of 34

Diallyl amine (1.04 g, 11 mol) was added to a solution of 31 (prepared from cbz-phenylcysteine to the oxrine²) (3.45 g, 10 mmol) in MeOH (50 mL) at rt and stirred for 24 h. The solvent was then removed in vacuo. The crude residue was dissolved in ethyl acetate (100 mL). The organic layer was washed with HCl (1N, 50 mL), water (50 mL), and brine (100 mL), dried over Na₂SO₄ and evaporated to dryness. The crude residue was purified on a column (silica gel, ethyl acetate:hexane, 60:40) to give cbz protected product 32 (3.4 g, 0.80%) as a foam.

Product 32 (2.13 g, 5 mmol), was dissolved in MeOH (50 mL) at ice bath temperature and anhydrous ammonia gas was bubbled through the reaction until saturated. The reaction allowed to warm to RT overnight. The solvent was then removed to give the crude product that was purified on a column (silica gel, MeOH:DCM:NH3, 90:10:0.1) to give pure 33 (1.03 g, 70%) as a pale foam.

To a solution of (3-amino-2-hydroxy-4-phenylsulfanyl-butyl)-diallyl amine (1 mmol) in THF at −10° C. was added 3-hydroxy-2-methylbenzoic acid (1.1 mmol), DCC (1.1 mmol), and HOBT (1.1 mmol). The mixture was allowed to warm to rt and stirred overnight. Solvent was removed in vacuo, EtOAc was added and the solid was filtered. The filtrate was washed with saturated sodium bicarbonate and brine, dried and concentrated. The residue was purified by flash chromatography to give pure diallyl amine-protected 34. The protecting groups were removed by the procedure described in reference 1 to give pure 34 at an overall yield of 60%.

### Example 22

Preparation of a Hapten Comprising Met-Sensitive Moiety (E4)

22.1 Preparation of 31

To a stirred solution of the 34 in DMF (2 mL) was added bromo acetyl NHS ester (130 mg, 0.6 mmol). The mixture stirred overnight and then diluted with water (10 mL). The mixture was then extracted with DCM (3×10 mL). The combined DCM layers were washed with brine (10 mL), dried (Na₂SO₄) and evaporated to dryness in vacuo. The crude was then purified on a column (silica gel, DCM:MeOH, 95:5) to give the bromoacetyl 35 (146 mg, 32%) as a white foam.

### Example 23

Preparation of a Hapten Comprising Met-Sensitive Moiety (E5)

23.1 Preparation of 36

To a stirred solution of the acid 29 (331 mg, 1 mmol) in DMF (2 mL) was added DCC (260 mg, 1.2 mmol) and NHS (120 mg, 1.4 mmol). The mixture was stirred for 6 h then amino disulfide (245 mg, 2 mmol) and DIEA (0.3 mL, 1.5 mmol) were added at rt and the reaction was stirred overnight. The solvent was then evaporated to dryness in vacuo. To the residue was added water (10 mL) and extracted with ethyl acetate (2×25 mL). The combined organic layer was then washed with HCl (1N, 4 mL), and saturated sodium bicarbonate (3 mL) and dried (Na₂SO₄). The ethyl acetate was removed under reduced pressure to give the crude product 36. The crude product was purified on a silica gel column (MeOH:hexane, 10:90) to give pure product 36 (218 mg, 50%) as a pale yellow solid.

### Example 24

Preparation of a Hapten Comprising Met-Sensitive Moiety (F2)

24.1 Preparation of 37

To a solution of N-[[N-Methyl-N-[(2-isopropyl-4-thiazolyl)methyl]amino]carbonyl]-L-valine (Xiamen MCHEM Pharma (Group) LTD., China) (1.0 g, 5 mmol) in DCM/DMF (25 mL/2 mL) was added DCC (1.13 g, 5.5 mmol), HOBT (0.74 g, 5.5 mmol) and phenylalanine t-butyl ester (1.21 g, 5.5 mmol) and DIEA (2.6 mL, 15 mmol). The mixture was stirred overnight, filtered and washed with 2.5% NaOH, 1N HCl, water, brine, dried over Na₂SO₄, filtered and concentrated to give 1.79 g of crude. The crude was purified on a silica gel column using EtOAc:hexanes (1:10 to 1:1) to give a mixture of 2 compounds. The yield was 1.04 g. 400 mg of this mixture was treated with TFA (3 mL) and stirred overnight. The TFA was then removed under reduced pressure. The crude product was purified on a silica gel column using DCM:MeOH:AcOH (95:5:0.3) to give 200 mg of N-[[N-Methyl-N-[(2-isopropyl-4-thiazolyl)methyl]amino]carbonyl]-L-valinyl-phenylalaine, 37.

### Example 25

Preparation of a Hapten Comprising Met-Sensitive Moiety (G1)

25.1 Preparation of 38

To a solution of 40 (0.3 g, 1.26 mmol) in DMF (5 mL) was added K₂CO₃ (0.35 g, 2.53 mmol) and tert-butyl bromoacetate (0.25 g, 1.28 mmol). The mixture was stirred at rt for 18 h. Water (20 mL) was added and extracted with EtOAc (2×25 mL), the organic layer was washed with water, brine, dried over Na₂SO₄ and concentrated to give 0.45 g of crude product which was purified on a silica gel column using EtOAc:hexanes (1:4) to yield 0.42 g. This product was treated with TFA (2 mL) for 1 h. The mixture was concentrated under vacuum to give 0.46 g of 3-tert-butylcarbamoyl-octahydro-isoquinolin-2-yl)-acetic acid, 38.

### Example 26

Preparation of a Hapten Comprising Met-Sensitive Moiety (G2)

26.1 Preparation of 39

To a solution of 40 (1 eq) in DCM was added K₂CO₃ (1.5 eq) and bromo acetyl bromide (1 eq). The reaction was stirred for 2 h, water was added and the organic layer was separated, dried over Na₂SO₄ and concentrated. The residue was purified by chromatography on silica gel to give pure 2-(2-bromo-acetyl)-decahydro-isoquinoline-3-carboxylic acid tert-butylamide, 39.

### Example 27

Preparation of a Hapten Comprising Met-Sensitive Moiety (G3)

27.1 Preparation of 41

To a stirred solution of 40 (474 mg, 2 mmol) in DMSO (3 mL) was added NaH (105 mg, 50% in oil, 2.1 mmol) at RT. The reaction was stirred for 1 hr and then a solution containing 4 mmol of TMS 4-bromobutyric acid TMS ester was added. TMS 4-bromobutyric acid TMS ester is prepared from 4-bromobutyric acid in DCM and TMSCl and imidazole and stirred for 6 h. Water (15 mL) and DCM (15 mL) were added to the reaction and the pH was adjusted to 3 by addition of HCl (1N). The aqueous layer was seperated and extracted with DCM (2×15 mL). The combined organic layers were washed with water (2×15 mL), brine (15 mL) and dried (Na₂SO₄). The solvent was removed under reduced pressure and the residue was purified on a column (silica gel, DCM:MeOH: AcOH; 95:5:0.1) to give 41 as a white foam (563 mg, 87%).

### Example 28

Preparation of a Hapten Comprising Met-Sensitive Moiety (G4)

28.1 Preparation of 42

A solution of 3 (1.0 g, 3.37 mmol) and decahydro-isoquinoline-3-carboxylic acid tert-butylamide 40 (0.8 g, 3.36 mmol) in dry 2-propanol (10 mL) was stirred under nitrogen and heated at 80° C. for 6 h. After cooling, the solvent was evaporated and the residue was purified by flash chromatography using EtOAc:hexanes (1:1) for the solution to give pure desired product. This was treated with TFA:DCM (1:1) overnight and then evaporated under vacuum to yield 2-(3-amino-2-hydroxy-4-phenyl-butyl)-decahydro-isoquinoline-3-carboxylic acid tert-butylamide, TFA salt, 40a.

To a solution of 40a (1 eq) in DMF was added K₂CO₃ and tert-butyl bromoacetate. After stirring at rt for 18 h, water was added, and the solution was extracted with EtOAc. The organic layer was then washed with water, brine, dried over Na₂SO₄ and concentrated to give the desired product which was purified by flash chromatography. The pure product was treated with TFA overnight. Evaporation of TFA gave [1-benzyl-3-(3-tert-butylcarbamoyl-octahydro-isoquinolin-2-yl)-2-hydroxy-propylamino]-acetic acid, 42.

### Example 29

Preparation of a Hapten Comprising Met-Sensitive Moiety (G5)

29.1 Preparation of 43

The above intermediate amine 40a (250 mg, 0.62 mmol) in THF (2 mL) and DIEA (260 μL, 1.5 mmol) was added choloro acetylbromide (195 μL, 0.62 mmol) over 2 h at ice bath temperature. The reaction was then warmed to rt and stirred for 1 hour. Water (10 mL) was added and the reaction mixture was extracted with DCM (3×15 mL). The combined organic layers were washed with brine (20 mL) and dried (Na₂SO₄) and evaporated to dryness to give the crude product that was purified (silica gel, DCM:MeOH, 95:5) to give the pure product N-[1-benzyl-2-hydroxy-3-(octahydro-isoquinolin-2-yl)-propyl]-2-bromo-acetamide, 43 (286 mg, 0.55%) as a pale solid.

### Example 30

Preparation of a Hapten Comprising Met-Sensitive Moiety (H11)

30.1 Preparation of 45

To a stirred solution of compound 44 (394 mg, 1 mmol) in DMF (2 mL) was added succinic anhydride (110 mg, 1.1 mmol) and DIEA (100 μL). 44 was prepared according to the procedure in Steve Turner et al. J. Med. Chem. 41:3467 (1998). The reaction mixture was stirred overnight at rt. Water (5 mL) was added to the reaction and the pH was adjusted to 3 by addition of HCl (1N). The mixture was then extracted with DCM (3×20 mL). The combined organic layer was washed with brine (20 mL), dried (Na₂SO₄) and evaporated to dryness in vacuo. The residue was further purified on silica gel (DCM:MeOH:AcOH; 95:5:0.1) to give 5,6-dihydro-4-hydroxy-3-(1-(3-aminophenyl)-6-phenyl-6-propyl-2H-pyran-2-one N-succinic acid, 45 (250 mg, 0.51 mmol, 51%) as a pale yellow solid.

30.2 Characterization Data for 45

¹H NMR (DMSO): 6.7-7.2 (m, 6H); 6.3 (m, 2H); 6.0 (m, 1H); 3.4 (m, 1H); 2.8 (t, 2H): 2.5 (m, 4H); 2.4 (t, 2H); 2.0 (m, 1H); 1.4-1.9 (m, 5H); 1.2 (m, 2H) and 0.70-0.9 (m, 6H).

### Example 31

Preparation of a Hapten Comprising Met-Sensitive Moiety (H2)

31.1 Preparation of 46

To a stirred solution of compound 44 (394 mg, 1 mmol) in DMF (2 mL) was added bromo acetyl NHS ester (260 mg, 1.1 mmol) and DIEA (100 μL). 44 was prepared according to the procedure in Steve Turner et al. J. Med. Chem. 41:3467 (1998). The reaction was stirred overnight. DCM (5 mL) and water (10 mL) was then added to the reaction mixture. The organic layer was separated and the aqueous layer was extracted once more with DCM (10 mL). The combined organic layer was washed with brine (2×10 mL) dried (Na₂SO₄) and evaporated under reduced pressure. The residue was purified on silica gel (DCM:MeOH:96:4) to give pure 5,6-dihydro-4-hydroxy-3-(1-(3-aminophenyl)-6-phenyl-6-propyl-2H-pyran-2-one N-bromoacetyl, 46 as a pale yellow solid (205 mg, 0.39 mmol, 39%).

31.2 Characterization Data for 46

¹H NMR (DMSO): 6.8-7.2 (m, 6H); 6.5 (m, 2H); 6.2 (m, 1H); 3.6 (m, 1H); 2.5 (m, 4H); 2.3 (s, 2H); 2.0 (m, 1H); 1.4-1.9 (m, 5H); 1.2 (m, 2H); 0.70-0.9 (m, 6H).

### Example 32

Preparation of a Hapten Comprising Met-Sensitive Moiety (I1)

32.1 Preparation of 48

To a stirred solution of efavirenz 47 (8 g, 25.34 mmol) in MeOH (100 mL) was added Lindlar's catalyst (7 g, 3.50 mmol). The suspension was stirred for one week under atmospheric pressure of hydrogen. The catalyst was then carefully filtered over celite and the filtrate was concentrated in vacuo to give the cis olefin 48 (8 g, 99%) as a white solid. The material was used for the next step without further purification.

32.2 Characterization Data for 48

¹H NMR (DMSO): 10.95 (s, 1H); 7.50 (dd, 1H); 7.38 (s, 1H); 6.98 (d, 1H); 5.90 (d, 1H); 5.40 (t, 1H); 1.30 (m, 1H); 0.80 (m, 1H); 0.45 (m, 2H) and 0.38 (m, 1H).

32.3 Preparation of 49

To a stirred solution of crude compound 48 (300 mg, 0.94 mmol) in DCM (5.9 mL) was added pyridine (590 μL) and OSO₄ (240 mg, 0.94 mmol) at rt for 1 h. To the stirred solution was then added an aqueous solution of NaHSO₃ (15%, 15 mL) and the reaction was stirred overnight. Water (10 mL) and DCM (50 mL) were added to the reaction mixture and the organic layer was separated. The aqueous layer was extracted with DCM (30 mL) once more. The combined organic layer was washed with water (15 mL), brine (15 mL), dried (Na₂SO₄) and concentrated under reduced pressure to give the crude product (290 mg) which was further purified (silica gel, EtOAc:hexane, 1:1) to give pure compound 49 (150 mg, 0.42 mmol, 45%)

32.4 Characterization Data for 49

¹H NMR (DMSO): 10.58 (s, 1H); 7.68 (s, 1H); 7.41 (dd, 1H); 6.76 (d, 1H); 6.39 (d, 1H); 4.77 (d, 1H); 3.95 (t, 1H); 2.89 (m, 1H); 0.96 (m, 1H); 0.06-0.26 (m, 4H).

32.5 Preparation of 50

To a stirred solution of diol 49 (150 mg, 0.41 mmol) in MeOH (4 mL) at ice bath temperature was added a saturated solution of NaIO₄ (5 mL) dropwise and the mixture was then stirred overnight at rt. The mixture was concentrated under the reduced pressure and the residue was partitioned between H₂O:EtOAc (70 mL, 2:5). The aqueous layer was separated and extracted with ethyl acetate (20 mL). The combined organic layer was dried (Na₂SO₄) and concentrated under reduced pressure to give crude 50 (150 mg). The crude compound was purified on silica gel (EtOAc:Hexane, 1:1) to give pure product 50 as a tan solid (72 mg, 0.23 mmol, 56%).

32.6 Characterization Data for 50

¹H NMR (DMSO): 10.75 (s, 1H); 7.54 (s, 1H); 7.50 (d, 1H); 7.44 (dd, 1H); 6.90 (d, 1H); 4.93 (d, 1H); and 3.35 (s, 3H).

32.7 Preparation of 52

To a stirred solution of hemiacetal 50 (70 mg, 0.22 mmol) in acetone (1 mL) at ice bath temperature was added dropwise a solution of Jones's reagent (100 μL). The reaction was then stirred for 1 hour at 4° C. and the partitioned between H₂O:EtOAc (30 mL, 1:2). The aqueous layer was separated and extracted with ethyl acetate (20 mL). The combined organic layer was washed with saturated solution of NaHCO₃ (10 mL), dried (Na₂SO₄) and concentrated under reduced pressure to give the methyl ester (45 mg). The methyl ester was hydrolyzed with a solution of KOH at pH 12 in MeOH (5 mL) overnight. The reaction mixture was evaporated to dryness under reduced pressure and then was partitioned between a water:ether (30 mL, 1:2). The organic layer was separated and acidified to pH 4 with HCl (1N). The acidified solution was then extracted with EtOAc (2×20 mL). The combined organic layer was washed with brine (10 mL), dried (Na₂SO₄) and evaporated to dryness to give 6-chloro-2-oxo-4-trifluoromethyl-octahydro-benzo[d][1,3]oxazine-4-carboxylic acid, 52 (23 mg, 0.08 mmol, 36%) that was used for the next step without further purification.

32.8 Characterization Data for 52

¹H NMR (DMSO): 11.23 (s, 1H); 10.92 (s, 1H); 7.60 (dd, 1H); 7.41 (s, 1H) and 7.02 (d, 1H).

### Example 33

Preparation of a Hapten Comprising Met-Sensitive Moiety (12)

33.1 Preparation of Intermediate

To a stirred solution of hemiacetal 50 (700 mg, 2.24 mmol) in dried acetonitrile (10 mL) was added methyl(triphenylphosphoranylidene)acetate (1.35 g, 4 mmol). The mixture was then refluxed for 2 h and was then stirred at rt overnight. The solvent was then removed in vacuo and the residue was purified on silica gel (EtOAc:Hexane, 1:3) to give the pure methyl ester (352 mg) that was hydrolyzed to unsaturated acid 53 without further purification.

33.2 Characterization Data for Intermediate

¹H NMR (CDCl₃): 8.82 (s, 1H); 7.28 (dd, 1H); 7.34 (s, 1H); 7.17 (d, 1H); 6.86 (d, 1H); 6.42 (d, 1H); and 3.82 (s, 3H).

33.3 Preparation of 53

To a stirred solution of the above unsaturated acid in MeOH (3 mL) was added Pd/C (10%, 100 mg) and hydrogenated at atmospheric pressure overnight. The mixture was filtered over Celite and concentrated in vacuo. The residue was hydrolyzed according to the procedure mentioned above and purified on silica gel (EtOAc:Hexane, 1:1) to give compound 53 (230 mg, 1.40 mmol, 62%) as a white solid.

33.4 Characterization Data for 53

¹H NMR (CDCl₃): 8.94 (s, 1H); 7.37 (dd, 1H); 7.22 (s, 1H); 6.84 (d, 1H); 2.60 (m, 3H); 2.36 (m, 1H).

### Example 34

Preparation of a Hapten Comprising Met-Sensitive Moiety (I3)

34.1 Preparation of 54

To a stirred solution of acid 52 (294 mg, 1 mmol) in DMF (1 mL) was added NHS (172 mg, 1.5 mmol) and DCC (193 mg, 1.1 mmol) at RT. The reaction mixture was stirred for 5 h and then ammonium hyroxide (2N, 0.2 mL) was added. The mixture was stirred overnight. Water (10 mL) was added and the mixture was extracted with DCM (2×20 mL). The combined organic layers were washed with brine (10 mL), dried (Na₂SO₄) and evaporated to dryness to give the corresponding amide (225 mg, 75%) as a pale foam that was used for the next step without further purification.

To a stirred solution of the amide (223 mg, 0.75 mmol) in THF (2 mL) and DIEA (260 μL, 1.5 mmol) at ice bath temperature was add dropwise bromoacetyl chloride (314 mg, 2 mmol). The reaction was stirred at ice bath temp for 1 hr and was then warmed to rt. Water (10 mL) was added and the milky solution was extracted with DCM (3×15 mL). The combined organic layers were washed with brine (20 mL) and dried (Na₂SO₄) and evaporated to dryness to give the crude product that was purified (silica gel, DCM:MeOH, 95:5) to give the pure product 54 (280 mg, 0.67%) as a pale yellow solid.

### Example 35

Preparation of a Hapten Comprising NNRTI Derivative (I4)

35.1 Preparation of 55

To a stirred solution of efavirenz 47 (500 mg, 1.5 mmol) in DMF (10 mL) was added methylacrylate (400 μL, 5.1 mmol) and potassium carbonate (600 mg, 4.2 mmol). The mixture was stirred for 72 h at rt. EtOAc (50 mL) was then added to the reaction and washed with water (10 mL), brine (10 mL) and dried (Na₂SO₄). The organic layer was then concentrated in vacuo and the residue was purified on silica gel (EtOAc:Hexane, 1:9) to give the methyl ester as pale yellow solid (320 mg). The ester was hydrolyzed to acide 55 by dissolving the methyl ester in a mixture of MeOH:H₂O (10 mL, 80:20) and K₂CO₃ (100 mg). The mixture was stirred overnight and then was acidified to pH 3 by addition of HCl (1N). The mixture was then extracted with ethyl acetate, dried (Na₂SO₄) and evaporated to dryness under reduced pressure to give acid 55 (275 mg, 85%) as a pale yellow solid.

### Example 36

Preparation of a Hapten Comprising NNRTI Derivative (I5)

36.1 Preparation of 56

To a srirred solution of efavirenz 47 (315 mg, 1 mmol) in DMSO (1 mL) was added NaH (53 mg, 50% in oil, 1.1 mmol) at RT. The mixture was stirred for 10 min and a solution of TMS 5-bromopentanoic ester (prepared from 5-bromopentanoic acid, TMSCl and imidazole in DCM, 2 mmol) was then added and the reaction was stirred for 6 h. Water (15 mL) and DCM (15 mL) was added to reaction and the pH was adjusted to 3 by addition of HCl (1N). The aqueous layer was seperated and extracted with DCM (2×15 mL). The combined organic layers were washed with water (2×1 5 mL), brine (15 mL) and dried (Na₂SO₄). The solvent was removed under reduced pressure and the residue was purified on a column (silica gel, DCM:MeOH: AcOH; 95:5:0.1) to give 56 as a yellow solid (249 mg, 60%).

### Example 37

Preparation of NNRTI Derivative (J1)

37.1 Preparation of 58

To a stirred solution of nevirapine 57 (500 mg, 1.87 mmol) in DMF (2 mL) was added K₂CO₃ (518 mg, 3.75 mmol) and methyl acrylate (338 μL, 3.75 mmol). The reaction mixture was stirred at rt overnight. Ethyl acetate (50 mL) and water (20 mL) was added to the reaction mixture and the organic layer was separated. The water layer was further extracted with ethyl acetate (2×50 mL) and combined. The combined organic layer was washed with water (2×20 mL), brine (20 mL), dried (Na₂SO₄) and concentrated under reduced pressure to give the crude methyl ester (710 mg) that was used for the next step without further purification.

To a stirred solution of the crude methyl ester in MeOH (5 mL) was added a solution of KOH (5N, 5 mL). MeOH was added dropwise to keep the mixture homogenous. The mixture was then stirred overnight. The mixture was evaporated to dryness in vacuo. To the residue was added water (20 mL) and extracted with ethyl acetate (2×30 mL). The aqueous layer was then acidified with HCl (1N) to pH 3 and extracted with ethyl acetate (5×50 mL) and CH₂Cl₂ (2×50 mL). The combined organic layer was dried (Na₂SO₄) and concentrated in vacuo to give the crude product (550 mg) which was further purified (silica gel, ethyl acetate:hexane, acetic acid, 4:1:0.1) to give the pure product 58 (300 mg, 0.88 mmol, 47%) as a white solid.

37.2 Characterization Data for 58

¹H NMR (DMSO): 12.13 (s, 1H); 8.41 (dd, 1H); 8.11 (dd, 1H); 7.98 (dd, 1H); 7.15 (dd, 1H); 7.11 (d, 1H); 4.68 (dt, 1H); 3.55 (m, 1H); 3.31 (dt, 1H); 2.38 (t, 2H); 2.31 (s, 3H); 0.83 (m, 2H); 0.66 (m, 1H); 0.34 (m, 1H).

### Example 38

Preparation of NNRTI Derivative (J2)

38.1 Preparation of 59

To a stirred suspension of nevirapine 57 (700 mg, 2.63 mmol) and K₂CO₃ (546 mg, 3.92 mmol) was added methyl 5-bromovalerate (420 mg, 2.63 mmol) in DMF (3.5 mL). The reaction was stirred at 120° C. overnight. The solvent was removed under high vacuum and the residue was partitioned between water and ether (70 mL, 2:5). The water layer was then evaporated and acidified with HCl (1N) to pH 4 and extracted with ethyl acetate (3×30 mL). The ethyl acetate layer was washed with brine (10 mL) and dried (Na₂SO₄). The solvent was then removed in vacuo to give the pure N-pentanoic acid nevirapine 59 (165 mg, 0.45 mmol, 17%) as a white solid.

38.2 Characterization Data for 59

¹H NMR (DMSO): 12.00 (s, 1H); 8.43 (dd, 1H); 8.14 (d, 1H); 8.00 (dd, 1H); 7.18 (dd, 1H); 7.14 (d, 1H); 4.41 (m, 1H); 3.58 (m, 1H); 3.06 (m, 1H); 2.30 (s, 3H); 2.11 (m, 2H); 1.35 (m, 4H); 0.90 (m, 2H) and 0.42 (m, 2H).

### Example 39

Preparation of NNRTI Derivative (J3)

39.1 Preparation of 60

To a stirred solution of Nevirapine 57 (532 mg, 2 mmol) in THF (10 mL) was added sodium hydride (101 mg in 50% oil, 2.1 mmol) at RT and under an atmosphere of argon. The mixture stirred for 10 min and then was cooled to ice bath temperature. 1,3-dibromoacetone (2.15 g, 10 mmol) was added. The ice bath was removed and the mixture was stirred for 6 h at RT. Water (10 mL) was added and the pH was adjusted to 5 by addition of HCl (1N). The mixture was extracted with ethyl acetate (3×30 mL). The combined organic layer was washed with brine (30 mL), dried (Na₂SO₄) and evaporated to dryness. The residue was purified on a column (silica gel, DCM: MeOH, 95:5) to give the desired product 60 (360 mg, 45%) as a pale yellow solid.

### Example 40

Immunogen Formation Involving Carboxylic Acids

(D2) is used in this Example. However, this conjugation technique is generally applicable to all met-sensitive moieties and NNRTI derivatives which are conjugated through a carboxylic acid moiety. The hapten is activated upon conversion of the carboxylic acid moiety to N-hydroxysuccinimide (NHS) ester. This Example specifically applies to compounds (D1), (D2), (G1), (G4), and (11).

A. Activation of (D2)

To a stirred solution of (D2) (10.7 mg, 30.8 mmol) in dried DMF (0.5 mL) was added 1-ethyl-3-(3-dimethylamino propyl)carbodiimide (EDAC) (5.7 mg, 29.7 mmol) and N-hydroxysuccinimide (NHS) (4.9 mg, 42.6 mmol) at ice bath temperatures. The mixture was stirred overnight. Ester formation was monitored by TLC analysis.

B. Conjugation of (D2) to KLH

Two vials of lyophilized KLH (Pierce, 27 mg per vial) were reconstituted with 2 mL of deionized water each and pooled. The mixture was allowed to stand overnight at 4° C. A buffer exchange was done by dialyzing overnight the KLH solution against 2 L of sodium bicarbonate buffer (0.1 M, pH 8.9). The final volume of the KLH preparation was 3.75 mL at a concentration of 14.4 mg/mL. A 1.2 mL aliquot of the KLH preparation (17.28 mg) was transferred into a reaction vial. The solution of Example 40A (320 μL) was then added slowly (10-20 μL per addition) to the solution of KLH over a period of 2 h at ice bath temperatures. After the addition was completed, the mixture was stirred in a 4° C. cold room overnight. This solution was then dialyzed against three changes (2.0 L each) of HEPES buffer (10 mM, pH 7.0, 1 mM). The final concentration of the KLH preparation was 4.5 mg/mL.

C. Conjugation of (D2) to Glucose-6-Phosphate Dehydrogenase

Lyophilized G6PDH (Worthington Biochem. Corp., 42.2 mg) was reconstituted with 3.5 mL deionized water to give a solution of 12.1 mg/mL. The mixture was allowed to stand overnight at 4° C. The mixture was then dialyzed overnight at 4° C. against 2 L of sodium bicarbonate buffer (0.1 M, pH 8.9). After dialysis, 0.6 mL (7.2 mg) of enzyme solution was transferred to a reaction vial.

The activated product of Example 40A was added in 5 to 10 μL quantities to a solution of glucose-6-phosphate dehydrogenase (G6PDH, 0.1 M in sodium carbonate buffer) glucose-6-phosphate (G6P, 4.5 mg/mg G6PDH), and NADH (9 mg/mg G6PDH) in a pH 8.9 sodium carbonate buffer at ice bath temperature. After the addition of each portion of solution of Example 40A a 2 μL aliquot was taken and diluted 1:500 with enzyme buffer. A 3 μL aliquot of this diluted conjugation mixture was assayed for enzymatic activity similar to that described in Example 47A below. The reaction was monitored and stopped at 59.3% deactivation of enzyme activity. The mixture was desalted with a PD-10 pre-packed Sephadex G-25 (Pharmacia, Inc.) and pre-equilibrated with HEPES buffer (10 mM, pH 7.0, 1 mM EDTA). The reaction mixture was applied to the column and the protein fractions pooled. The pooled fractions were dialyzed against three (1.0 L each) changes of HEPES (10 mM, pH 7.0, 1 mM EDTA) to yield a solution of the conjugate.

D. Determination of the Number of Met-Sensitive Moieties on an Immunogenic Carrier

KLH conjugated product from Example 40B buffer were dialyzed against bicarbonate buffer (0.1 M, pH 8.5). A series of known concentrations of glycine standards (Pierce) ranging from 2 to 20 μg/mL were prepared in bicarbonate buffer (0.1 M, pH 8.5). 0.25 mL of the 0.01% (w/v) solution of 2,4,6-trinitrobenzene sulfonic acid (Pierce, TNBS) was added to 0.5 mL of each sample solution and mixed well. Reaction mixture was incubated at 37° C. for 2 h. After the mixture was cooled to rt, 0.25 mL of 10% sodium dodecyl sulfate (SDS) and 0.125 mL of 1 N HCl was added to each sample. The absorbance of the sample and standard solutions at 340 nm were measured, and the quantitative determination of the number of amines contained within a KLH sample was accomplished through comparison to a glycine standard curve, according to the method of given in Bioconjugate Techniques, p. 112-113, 1966, Academic Press, San Diego, Calif., incorporated herein by reference. The number of haptens conjugated to KLH was determined to be 1,500.

### Example 41

Immunogen Formation Involving Halogens

(G5) is used in this Example. However, this conjugation technique is generally applicable to all met-sensitive moieties and NNRTI derivatives which are conjugated through a bromine moiety. This Example specifically applies to compounds (G2) and (G5).

A. Activation of KLH

One vial of lyophilized KLH (Pierce, 27 mg) was reconstituted with 1 mL of deionized water. This KLH solution was dialyzed against phosphate buffer (0.1 M, 0.15 M NaCl, 1 mM EDTA, pH 8.0). The dialyzed KLH was transferred to a reaction vial. 2-Iminothiolane (2-IT) (Pierce, 4.0 mg, 29.1 μmol) was dissolved in water to give a 2 mg/mL solution. The 2-IT solution was added to KLH with stirring. After 75 min, the mixture was desalted with a PD-10 pre-packed Sephadex G-25 (Pharmacia, Inc.) and then pre-equilibrated with phosphate buffer (100 mM, pH 8, 1 mM EDTA) to remove excess 2-IT.

B. Procedure for Quantitating Sulfhydryl Groups Using a Cysteine Standard

Cysteine standards ranging from 0 to 1.5 mM were prepared by dissolving cysteine hydrochloride monohydrate in Reaction Buffer (0.1 M sodium phosphate, pH 8.0, containing 1 mM EDTA). A set of test tubes were prepared, each containing 50 μL of Ellman's Reagent Solution (Pierce, dissolve 4 mg Ellman's Reagent in 1 mL of Reaction Buffer) and 2.5 mL of Reaction Buffer. 250 μL of each standard or KLH was added to the separate test tubes. KLH samples were appropriately diluted so that the 250 μL sample applied to the assay reaction has a sulfhydryl concentration in the working range of the standard curve. The reaction mixture was incubated at room temperature for 15 min. The absorbance was measured at 412 nm. The values obtained for the standards were plotted to generate a standard curve. KLH sample concentrations were determined from the curve.

C. Conjugation of Thiolated KLH to (G5): Formation of an Immunogen

Dithiothreitol (DTT, 5 mM, 2.3 mg) was added to thiolated KLH. The solution was allowed to mix overnight at 4° C. (G5) (9.3 mg, 21.7 pmol) was dissolved in 0.5 mL DMF. After stirring for 1 h, the dissolved product was added in 5 to 10 μL quantities to a solution of thiolated KLH from Example 41A. The solution comprising (G5) was added until a slight precipitation was observed. The reaction was continued overnight at 4° C. This solution was dialyzed against three changes (2.0 liter each) of HEPES buffer (10 mM, pH 7.0, 1 mM EDTA). The final volume of the KLH preparation was 3.5 mL at a concentration of 7.7 mg/mL.

### Example 42

Immunogen Formation Involving Amines

This conjugation technique is generally applicable to all met-sensitive moieties and NNRTI derivatives which are conjugated through an amine moiety. This Example specifically applies to compounds (D3), (A2), and (E3).

A. Activation of KLH: Succinylation

Lyophilized succinylated KLH (Sigma, 11 mg) was reconstituted with 2 mL deionized water. The KLH solution was dialyzed overnight two changes (2.0 L each) MES buffer (0.1 M MES, 0.9 M NaCl, 0.02% NaN₃, pH 4.7). After dialysis 6 mg of succinylated KLH was transferred to a reaction vial. (D3) (3.7 mg, 11.1 μM) was dissolved in dry DMF and added to the reaction vial slowly. EDC (Pierce, 10 mg) was dissolved in 1 mL deionized water and immediately add 50 μL of this solution to the KLH-(D3) solution. Additional EDC aliquots (10 μL per addition) were added until slight precipitation occurred during the conjugation reaction. The reaction was allowed to proceed for approximately 2 h under constant mixing at room temperature. The reaction mixture was dialyzed was then dialyzed against three changes (2.0 L each) of HEPES buffer (0.05 M, pH 7.2, 1 mM EDTA).

### Example 43

Immunogen Formation Involving Sulfhydryls

Met-sensitive moiety (E5) is used in this Example. However, this conjugation technique is generally applicable to all PIs and NNRTIs which are conjugated through a sulfhydryl moiety.

A. Conjugation of (E5) to Bromoacetylated G6PDH

50 μL DMF of was added to bromoacetic acid NHS (Sigma 3.06 mg, 12.97 μM) and stirred. A 2.0 mL (10 mg/mL) G6PDH solution was prepared in 0.05 M Tris HCl buffer, pH 8.2. 45 mg disodium G6P and 90 mg NADH, was dissolved in the G6PDH solution. Bromoacetic acid NHS was added to G6PDH solution at 5 μL increments. Enzyme activity was measured on the Cobas Mira analyzer after each addition. Bromoacetic acid NHS was added until 63.6% enzyme deactivation was obtained. G6PDH conjugation solution was dialyzed with 3×4 liter portions of 0.01 M phosphate, pH 7.2.

(E5) (3.0 mg, 6.87 μM) was dissolved in 125 μL carbitol, plus 6.5 μL 20 mM acetate buffer, pH 4.5. Carbitol and buffer were degassed before use. TCEP HCl was added (2.0 mg, 6.98 μM) and mixed for 2 h. TLC showed complete reduction of (E5) when it was sprayed with Ellman's reagent.

To the G6PDH solution 5 μL increments of (E5) solution was added. The total addition took less than 1 hour. Conjugation was reacted overnight at 4° C. The solution was transferred to a dialysis bag and dialyzed with 3×4 liter portions of 0.01 M phosphate, pH 7.2, at 4° C.

### Example 44

Preparation of Monoclonal Antibodies reactive to Met-Sensitive Moiety (D2)

A. Hybridoma Production

Standard hybridoma procedures used have been described in detail (Kohler, G. et al., Nature 256: 495-497 (1976); Hurrell, Monoclonal Hybridoma Antibodies: Techniques and Applications, CRC Press, Boca Raton, Fla. (1982)). This hybridoma technique is generally applicable to produce monoclonal antibodies to the met-sensitive moieties and NNRTI derivatives of the invention.

5 mice (Balb/c) were immunized with an immunogen comprising Met-Sensitive Moiety (D2) and KLH (“Immunogen (D2)/KLH”) according to the schedule shown in Table 2.

TABLE 2
Immunization Schedule

| Immunization | Immunogen | Amount | Adjuvant | Delivery |
|---|---|---|---|---|
| Initial | Immunogen | 100 μg | FCA | ip |
| | (D2)/KLH | | | |
| 2 week | Immunogen | 100 μg | FIA | ip |
| | (D2)/KLH | | | |
| 4 week | Immunogen | 100 μg | FIA | ip |
| | (D2)/KLH | | | |
| 8 week | | | | |
| Day - 3 | Immunogen | 100 μg | HBSS | sc |
| | (D2)/KLH | | | |
| Day - 2 | Immunogen | 100 μg | HBSS | sc |
| | (D2)/KLH | | | |
| Day - 1 | Immunogen | 100 μg | HBSS | sc |
| | (D2)/KLH | | | |

At the end of this immunization schedule, mice were sacrificed and the spleens removed and were ready for fusion to myeloma cells. The parental myeloma line used for all fusions was P3X63 Ag 8.653. Approximately 3-3.5×10⁷ myeloma cells per spleen were spun down at 800 rpm for 8 min, then resuspended in 20 mL of DMEM. The excised spleens were cut into small pieces, gently crushed in a tissue homogenizer containing 7 mL DMEM, then added to the myeloma cells. The cell suspension was spun down at 800 rpm for 8 min and the supernatant poured off. The cells were resuspended in 2 mL/spleen 50% aqueous polyethylene glycol solution added over a 3-min period with gentle swirling, then 1 mL/spleen DMEM was added over a 1.5 min period, and 5 mL/spleen Super DMEM was added over an additional 1.5 min period. The cells were spun down at 800 rpm for 8 min, the supernatant poured off, and the cells resuspended in HAT media, approximately 100 mL/spleen. The fusing cells were then plated out into four to six 96-well plates per spleen and placed in a CO₂ incubator. The plates were fed with HAT media on Day 7, with HT media on Day 10 and were screened on Day 12.

All cells were cloned and grown in macrophage-conditioned media. This media was made by injecting 10 mL of Super DMEM into the peritoneal cavity of an euthanized mouse. Macrophage cells were loosened by tapping the outside of the cavity, and the media was withdrawn and added to 200 mL of Super DMEM. The cells were allowed to grow in a CO₂ incubator for 3-4 days, then the media was filtered through a 0.22 μm filter to remove all cells. The supernatant was mixed with 350 mL of additional Super DMEM. This resultant “macrophage-conditioned” media was stored at 4° C. It was used within one month. Cloned lines were frozen down and stored at −100° C. in 10% DMSO (in Super DMEM).

Monoclonal antibody subclasses were determined using a variety of mouse monoclonal antibody isotyping kits, most frequently those by Southern Biotechnology and Zymed. All are ELISA based, and culture supernatant and manufacturer's instructions were followed.

B. Primary Screening

The primary fusion screen was a reverse ELISA procedure which was set up such that the monoclonal antibody is bound on the Enzyme Immunoassay (EIA) plate by rabbit anti-mouse Ig serum, and positive wells are selected by their ability to bind enzyme conjugates of the specific drug in question. The fusion was initially screened with the Met-Sensitive (D2)/G6PDH Enzyme conjugate described in Example 41 C. Positives from these primary screens were transferred to 24-well plates, allowed to grow for several days, then were screened by a competition reverse ELISA, wherein the enzyme conjugate must compete with free drug i.e., lopinavir, for antibody binding sites. If the enzyme activity measured when free drug was present was less than that seen when only enzyme conjugate is present, then the antibody preferentially binds the free drug over the enzyme conjugated form. Screening duplicate plates involving several different free drug solutions gave an indication of relative preference for each of the drugs. Selected wells from the competition screen were cloned by serial dilution at least four times, with cloning plates screened by reverse ELISA; occasional competition reverse ELISAs were used to eliminate more monoclonal antibodies during the cloning process.

C. Secondary Screening

Positives from the primary screen were also tested on a Cobas Mira analyzer for inhibition of enzyme conjugate and cross-reactivity with various free drug solutions in the homogeneous enzyme immunoassay configuration. Selected monoclonal antibodies were again tested for modulation and cross-reactivity and eliminated from consideration.

D. Selected Antibody Scale-Up

Clones that were selected as acceptable according to primary and second antibody screening were used in scaling up antibody production. This scale up was performed in ascites. The mice were primed by an ip injection of FIA to induce tumor growth, 0.3 to 0.5 mL/mouse, 2 to 7 days prior to passage of cells. Cells were grown up in log phase in a T-75 flask, about 18×10⁶ cells, centrifuged, and then resuspended in 2 mL of S-DMEM. Each mouse received a 0.5 mL ip injection of approximately 4-5×10⁶ cells. An ascites tumor usually developed within a week or two. The ascites fluid containing a high concentration of antibody was then drained using an 18-gauge needle. The fluid was allowed to clot at room temperature and then centrifuged at 1500 rpm for 30 min. The antibody containing fluid was poured off and stored frozen at −20° C.

### Example 45

Preparation of Polyclonal Antibodies Reactive to Met-Sensitive Moiety (D2)

This technique is generally applicable to produce polyclonal antibodies to the met-sensitive moieties and NNRTI derivatives of the invention.

Polyclonal sera from a live rabbit was prepared by injecting the animal with an immunogenic formulation. This immunogenic formulation comprised 200 μg of the immunogen for the first immunization and 100 μg for all subsequent immunizations. Regardless of immunogen amount, the formulation was then diluted to 1 mL with sterile saline solution. This solution was then mixed thoroughly with 1 mL of the appropriate adjuvant: Freund's Complete Adjuvant for first immunization or Freund's Incomplete Adjuvant for subsequent immunizations. The stable emulsion was subsequently injected subcutaneously with a 19×1½ needle into New Zealand white rabbits. Injections were made at 3-4 week intervals. No anesthesia was used. Bleeds of the immunized rabbits were taken from the central ear artery using a 19×1 needle. Blood was left to clot at 37° C. overnight, at which point the serum was poured off and centrifuged. Finally, preservatives were added in order to form the polyclonal antibody material. Rabbit polyclonal antibodies to lopinavir Met-Sensitive Moiety (D1) produced by the above procedure are designated Anti-(D1)1 and Anti-(D1)2, and polyclonal antibodies to lopinavir Met-Sensitive Moiety (D2) are designated Anti-(D2)1 and Anti-(D2)₂.

### Example 46

Selection of Enzyme Conjugates and Antibodies

This technique is generally applicable to select for enzyme conjugates comprising the met-sensitive moieties and NNRTI derivatives of the invention. This technique is also generally applicable to select for antibodies raised against the met-sensitive moieties and NNRTI derivatives of the invention.

Enzyme Conjugates comprising Met-Sensitive Moiety (D2) and G6PDH (“Enzyme Conjugate (D2)/G6PDH”), as well as Met-Sensitive Moiety (D1) and G6PDH (“Enzyme Conjugate (D1)/G6PDH”) were prepared according to Example 40. The binding of (D2) to G6DPH reduced the activity of the enzyme, and thus its Max Inhibition level, by 64.2% over the pre-conjugate activity level. The binding for (D1) to G6PDH reduced the enzyme activity of the enzyme, and thus its Max Inhibition level, by 52.3% over the pre-conjugate activity level. The Enzyme Conjugates were each included in a reagent mixture (“Enzyme Conjugate (D1))/G6PDH Reagent” and “Enzyme Conjugate (D2)/G6PDH Reagent”). These mixtures contained the enzyme conjugate, HEPES buffer, bulking agents, stabilizers, and preservatives.

G6PDH activity in the Enzyme Conjugates was optimized to give an enzymatic reaction rate (ODₘₐₓ) of 550 mA/min. The optimized activity is referred to as ODₘₐₓ. ODₘₐₓ represents the maximum optical density (signal) which the signal producing system can generate under the assay conditions. ODₘₐₓ is determined by measuring the optical density produced by combining the specified amount of each conjugate with the specified amounts of the other components of the signal producing system in the absence of antibody.

Antibodies evaluated for percent inhibition against this enzyme conjugate included Anti-(D1)1, Anti-(D1)₂, Anti-(D2)1, and Anti-(D2)₂ of Example 45. Key selection factors included maximum inhibition of enzyme conjugate and reduction in inhibition by addition of lopinavir.

TABLE 3
Max Inhibition of G6PDH in Immunogen
When Combined With Antibody

| | Percent Anti-Fragment Antibody Max Inhibition | | | |
|---|---|---|---|---|
| | Anti-(D2)1 | Anti-(D2)2 | Anti-(D1)1 | Anti-(D1)2 |
| Enzyme | 38.2 | 41.5 | 52.9 | 54.9 |
| Conjugate (D1) | | | | |
| Enzyme | 65.6 | 62.7 | 67.2 | 59.0 |
| Conjugate (D2) | | | | |

### Example 47

Immunoassay for Lopinavir in Serum Samples

A. Materials and Methods

This technique is generally applicable to select for immunoassays involving the met-sensitive moieties and NNRTI derivatives of the invention.

Enzyme Conjugate (D2)/G6PDH and Antibody Anti-(D2)₂ were selected as exemplary materials for the development of a homogeneous enzyme immunoassay for the anti-HIV therapeutic lopinavir. Enzyme Conjugate (D2)/G6PDH Reagent, as described in Example 45, was used. Also antibody Anti-(D2)₂ was used in this Example as part of an antibody reagent (“Anti-(D2)₂ Antibody Reagent”) further comprising nicotinamide adenine dinucleotide, glucose-6-phosphate, sodium chloride, bulking agent, surfactant, and preservatives.

An immunoassay for lopinavir was conducted on the Cobas Mira Chemistry Analyzer (Roche). On the analyzer, 4 μL of sample plus 61 μL water were incubated for 300 sec with 150 μL of Anti-(D2)₂ Antibody Reagent. Subsequently, 75 μL of the Enzyme Conjugate (D2)/G6PDH Reagent was added. After 25 sec incubation, enzyme activity was monitored by following the production of NADH spectrophotometrically at 340 nm for 50 sec.

B. Assay Performance

B. i) Standard Curve

A series of known concentrations of lopinavir standards (ranging from 0 to 10 μg/mL) were prepared gravimetrically in MES (2-(N-Morpholino)ethanesulfonic acid, 0.01 M, pH 5.5) formulated with EDTA, protein additive, detergent, antiform agent, and preservative. Similarly, quality control samples were prepared (1.0 and 5.0 μg/mL).

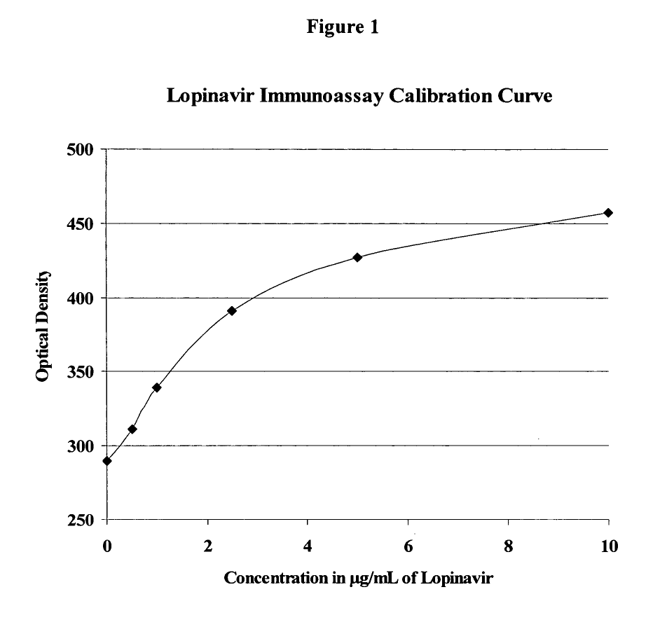

Lopinavir was dissolved in methanol to give a stock solution of 1000 μg/mL. Synthetic buffered calibrator matrix 10 mL aliquots were spiked to give lopinavir standards with concentrations shown in Table 4. A series of Anti-(D2)₂ Antibody Reagents were prepared by adding antibody to antibody/substrate diluent. Each antibody/substrate reagent was assayed with Enzyme Conjugate (D2)/G6PDH Reagent. Calibration curves were generated on the Cobas Mira by assaying each level in duplicate. An example of these calibration curves is provided in FIG. 1.

TABLE 4

| Lopinavir Concentration | Reaction Rate |
|---|---|
| (μg/mL) | (mA/min) |
| 0.0 | 289.9 |
| 0.5 | 311.1 |
| 1.0 | 338.9 |
| 2.5 | 390.7 |
| 5.0 | 427.0 |
| 10.0 | 457.1 |

B. ii) Within-Run precision

Human serum samples spiked with known concentrations of lopinavir were used to assess within-run precision. A stock solution of lopinavir was prepared by dissolving lopinavir in methanol to give a stock solution of 1000 μg/mL. Negative HIV therapeutic pooled human serum was spiked to give a final nominal concentration of 1.0 and 5.0 μg/mL. Determinations were performed by assaying 20 replicates at each of two levels. Quantification was performed on the Cobas Mira analyzer.

TABLE 5
Within-run Precision

| | Spiked | | | |
|---|---|---|---|---|
| | Level | Mean | | |
| N | (μg/mL) | (μg/mL) | SD | CV (%) |
| 20 | 1.0 | 0.92 | 0.04 | 4.35 |
| 20 | 5.0 | 4.96 | 0.27 | 5.44 |

B. iii) Analytical Recovery

The human serum samples spiked with known concentrations of lopinavir, as described in part B. ii) above, were also used to assess analytical recovery. A stock solution of lopinavir was prepared by dissolving lopinavir in methanol to give a stock solution of 1000 μg/mL. Ten individual HIV drug negative human serum samples were split into two 1 mL sample sets. One set of ten samples was spiked to give a nominal concentration of 1 and the other set of 10 samples spiked to give a nominal concentration of 5 μg/mL. Each sample was assayed in duplicate on the Cobas Mira analyzer. Averaged data is provided in Table 6.

TABLE 6
Analytical Recovery Data Summary

| Spiked Level | Mean | Recovery |
|---|---|---|
| (μg/mL) | (μg/mL) | (%) |
| 1.0 | 1.01 | 101.3 |
| 5.0 | 4.78 | 95.6 |

B. iv) Specificity of the Immunoassay

The specificity of the immunoassay was evaluated by adding potentially crossreactant drugs to human serum and determining the increase in the apparent concentration as a result of the presence of crossreactant. Separate stock solutions of lopinavir, ritonavir, amprenavir, saquinavir, indinavir, nelfinavir and efavirenz were prepared by dissolving the drug in methanol to give a stock solution of 1000 μg/mL. 10 μg/mL of crossreactant plus 5 μg/mL of lopinavir was added to individual human serum samples to give a final volume of 1 mL. Each sample was assayed in duplicate. Testing was performed on the Cobas Mira analyzer. The percentage concentration above 5 μg/mL of lopinavir was calculated for each crossreactant.

TABLE 7
Cross-Reactivity of Antibody with other
PIs and NNRTIs used in anti-HIV therapy

| | Percent Increase in |
|---|---|
| | Apparent Lopinavir |
| Sample | Conc. above 5 μg/mL |
| Ritonavir 10 μg/mL + 5 μg/mL Lopinavir | 0% |
| Amprenavir 10 μg/mL + 5 μg/mL Lopinavir | 1% |
| Saquinavir 10 μg/mL + 5 μg/mL Lopinavir | 1% |
| Indinavir 10 μg/mL + 5 μg/mL Lopinavir | 0% |
| Nelfinavir 10 μg/mL + 5 μg/mL Lopinavir | 1% |
| Efavirenz 10 μg/mL + 5 μg/mL Lopinavir | 0% |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method for determining, in a sample from a host, the concentration of an anti-HIV therapeutic which inhibits HIV propagation, wherein said anti-HIV therapeutic is selected from the group consisting of a HIV protease inhibitor (PI) and a non-nucleoside HIV reverse transcriptase inhibitor (NNRTI) and said anti-HIV therapeutic comprises a metabolically-sensitive (“met-sensitive”) moiety that is transformed by the host to yield an inactivated metabolic product,
said method comprising:
(a) combining, in a solution, said sample with an antibody specific for said met-sensitive moiety where the antibody does not bind to said inactivated metabolic product, thus yielding an antibody-anti-HIV therapeutic complex; and
(b) detecting said complex.

2. The method of claim 1, wherein said antibody further comprises a non-isotopic signal-generating moiety.

3. The method of claim 1, wherein said PI is selected from the group consisting of amprenavir, atazanavir, indinavir, lopinavir, nelfinavir, ritonavir, saquinavir, and tipranavir.

4. The method of claim 1, wherein said NNRTI is selected from the group consisting of efavirenz, nevirapine, delavirdine, and loviride.

5. The method of claim 1, wherein said method is a homogeneous immunoassay.

6. The method of claim 5, wherein said detecting further comprises:
mixing said solution containing said antibody-anti-HIV therapeutic complex with a conjugate comprising said met-sensitive moiety and a non-isotopic signal generating moiety;
measuring the amount of said antibody bound to said conjugate by monitoring a signal generated by said non-isotopic signal generating moiety; and
correlating said signal with the presence or amount of said anti-HIV therapeutic in said sample.

7. The method of claim 6, wherein said signal generating moiety is selected from the group consisting of an enzyme, a fluorogenic compound, a chemiluminescent compound, and combinations thereof.

8. The method of claim 7, wherein said enzyme is glucose-6-phosphate dehydrogenase.

9. The method of claim 1, wherein said met-sensitive moiety is a member selected from:

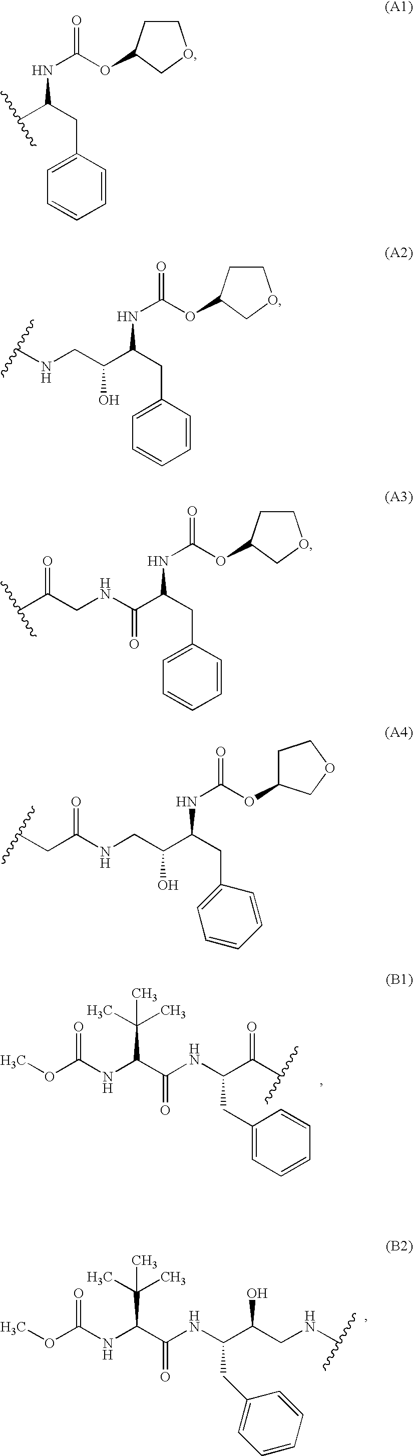

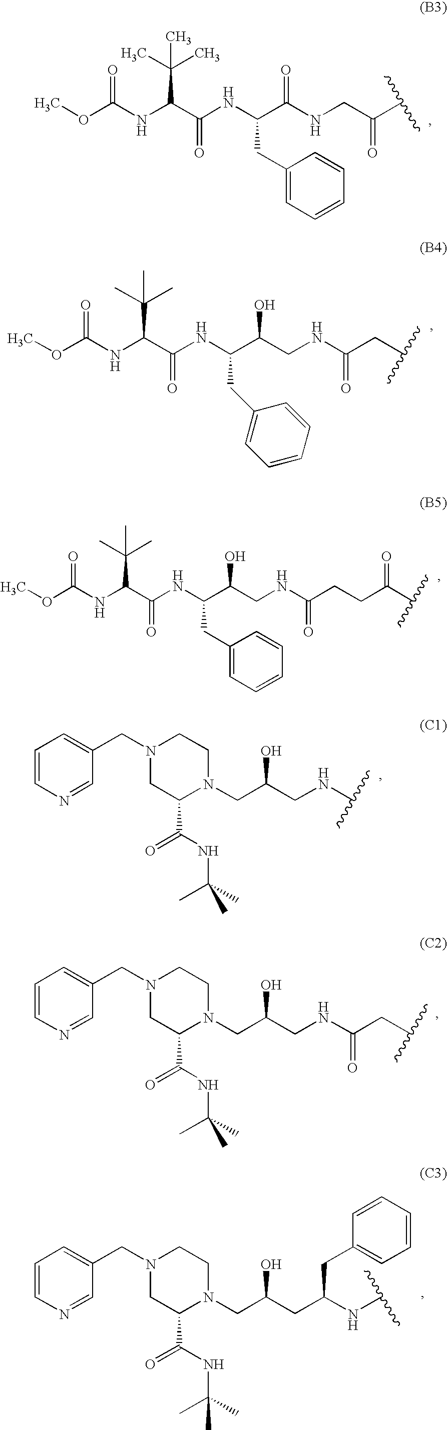

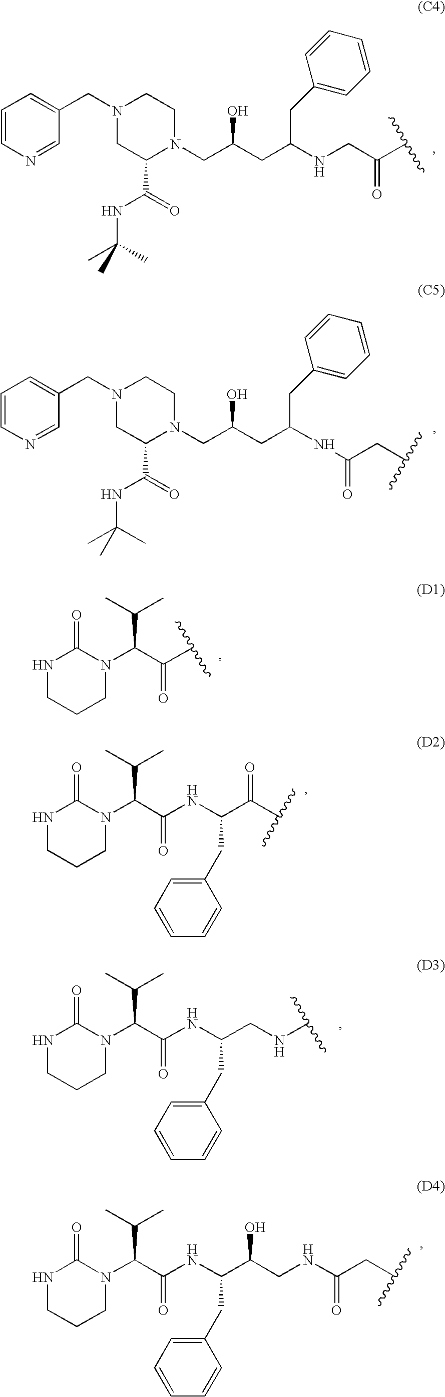

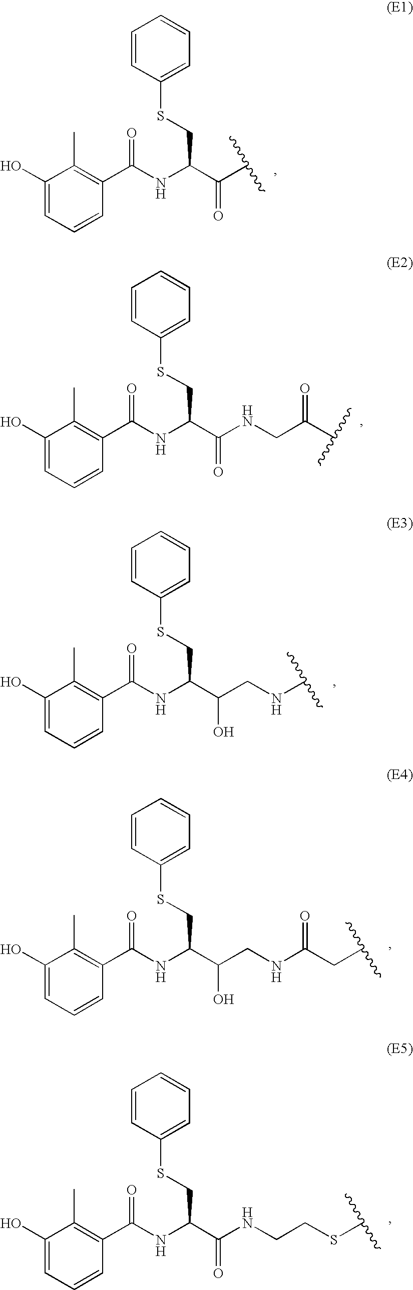

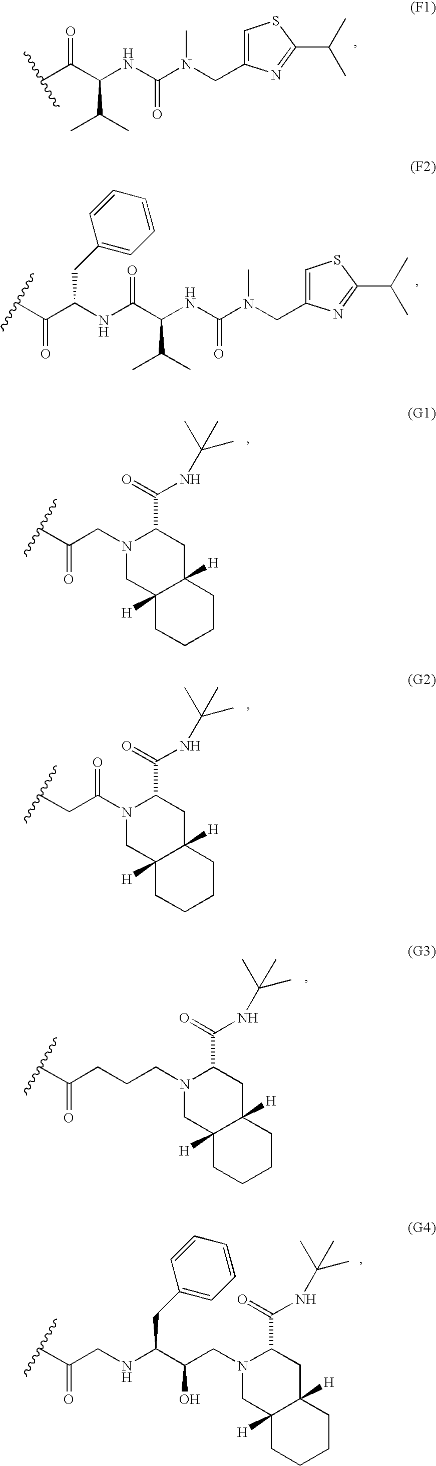

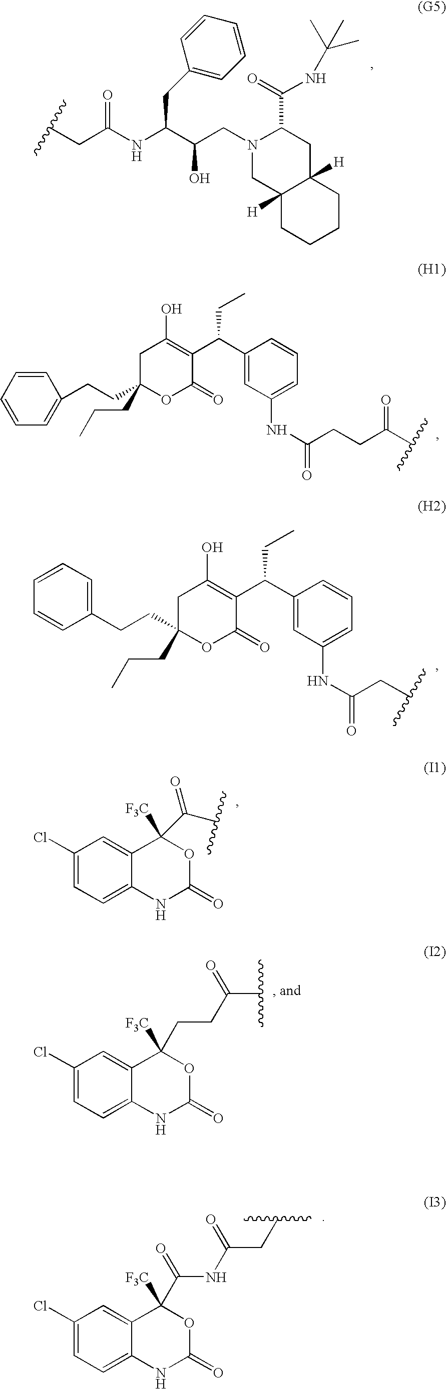

10. A compound having the structure:
I—(X)ₖ—(C═O)ₘ—(Y)ₙ-(L)ₚ-Q
wherein I is a met-sensitive moiety of an anti-HIV therapeutic, wherein said anti-HIV therapeutic is selected from the group consisting of a HIV protease inhibitor (PI) and a non-nucleoside HIV reverse transcriptase inhibitor (NNRTI);
X is selected from the group consisting of O, NH, and CH₂;
Y is selected from the group consisting of O, NH, CH₂, and CH₂—S;
k, m, n, and p are independently selected from 0 and 1;
L is a linker consisting of from 1 to 40 carbon atoms arranged in a straight chain or a branched chain, saturated or unsaturated, and containing up to two ring structures and 0-20 heteroatoms, with the provision that not more than two heteroatoms may be linked in sequence; and
Q is a reactive functional moiety chosen from the group consisting of active esters, halogens, isocyanates, isothiocyanates, thiols, imidoesters, anhydrides, maleimides, thiolactones, diazonium groups and aldehydes.

11. The compound of claim 10, wherein said PI is selected from the group consisting of amprenavir, atazanavir, indinavir, lopinavir, nelfinavir, ritonavir, saquinavir, and tipranavir.

12. The compound of claim 10, wherein said NNRTI is selected from the group consisting of efavirenz, nevirapine, delavirdine, and loviride.

13. The compound of claim 11, wherein said I is a member selected from:

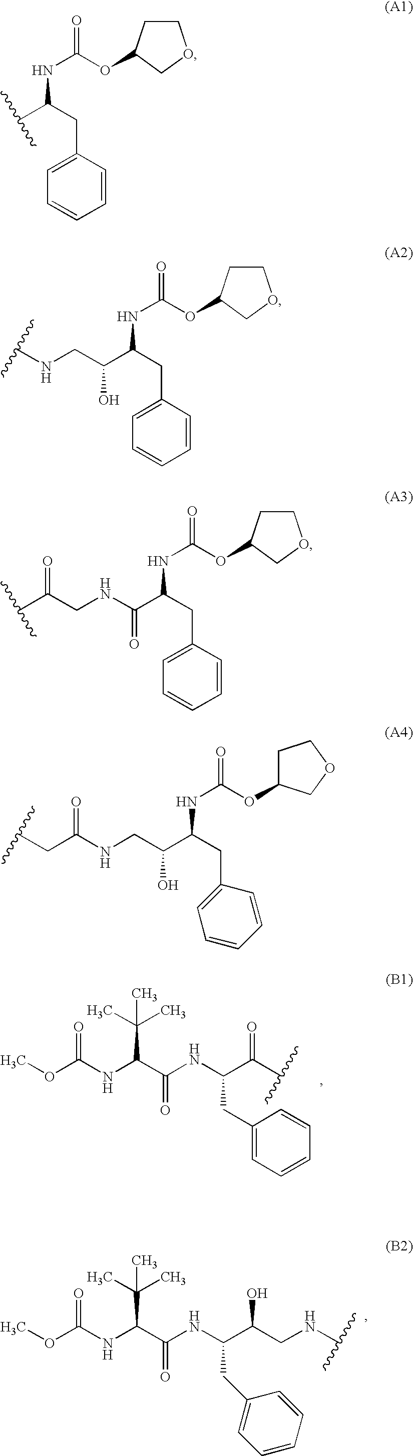

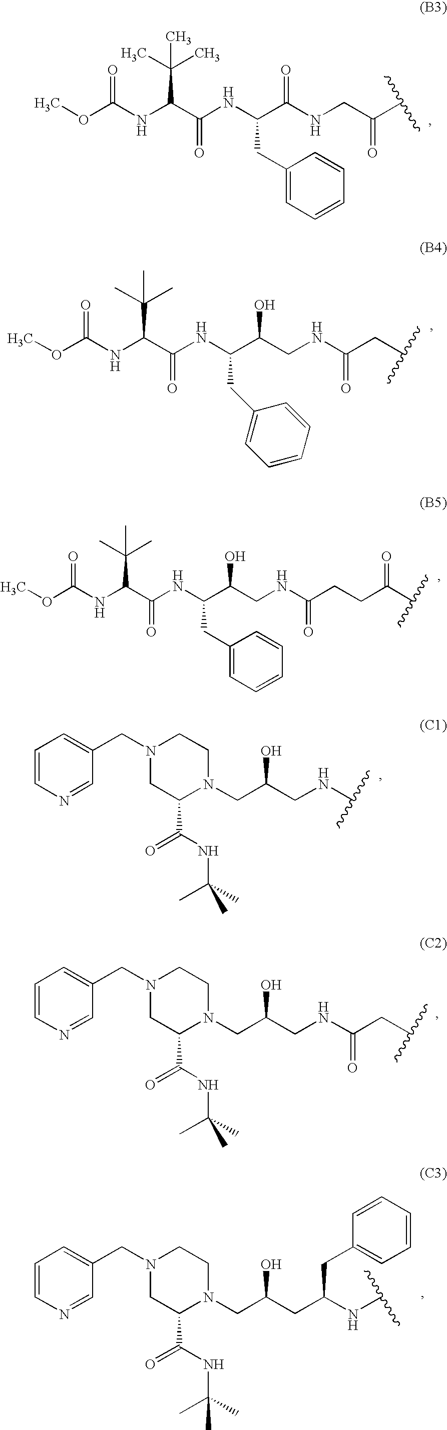

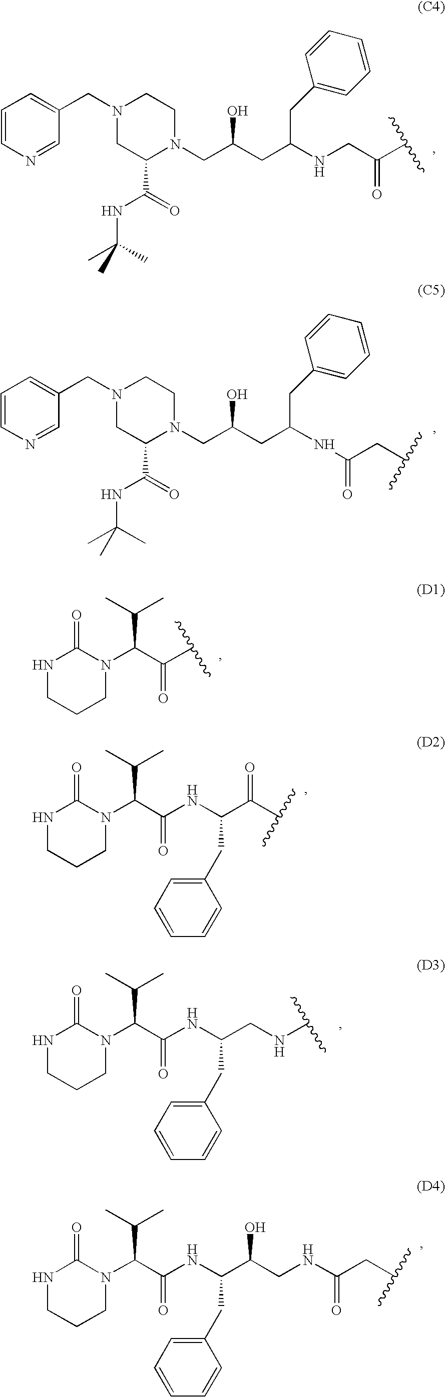

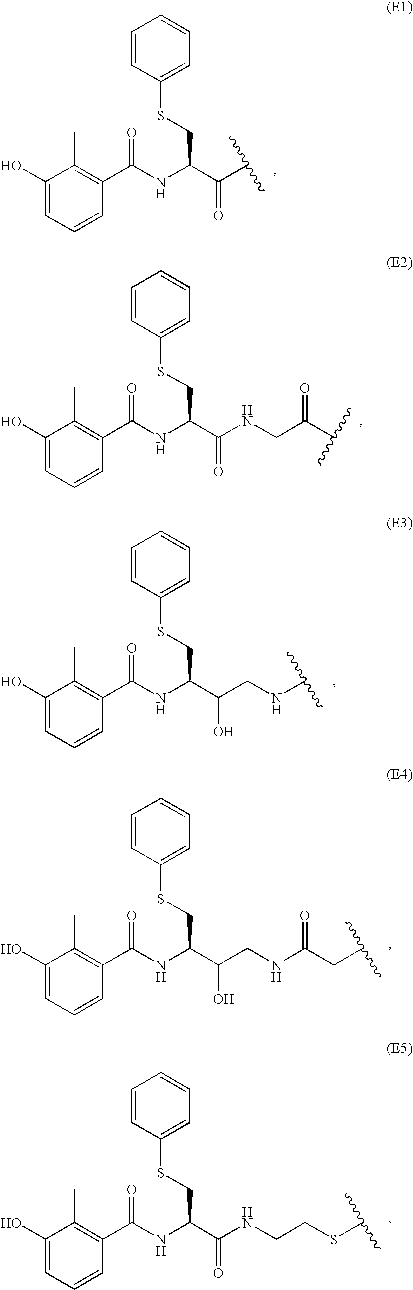

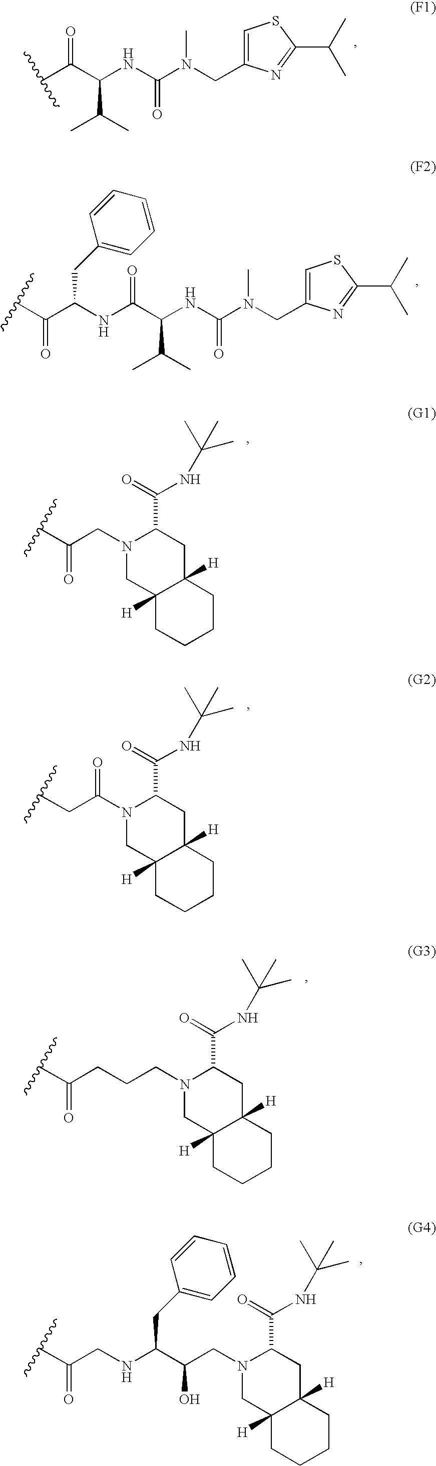

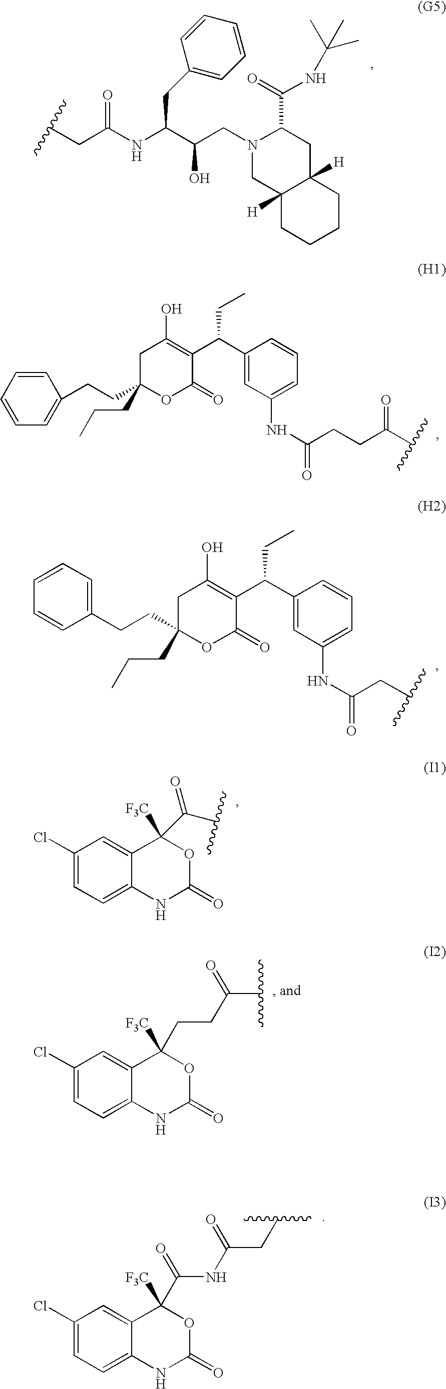

14. The compound of claim 10, wherein k is 1, X is O, m is 0, n is 0, p is 0, Q is succinimide, and I is a member selected from:

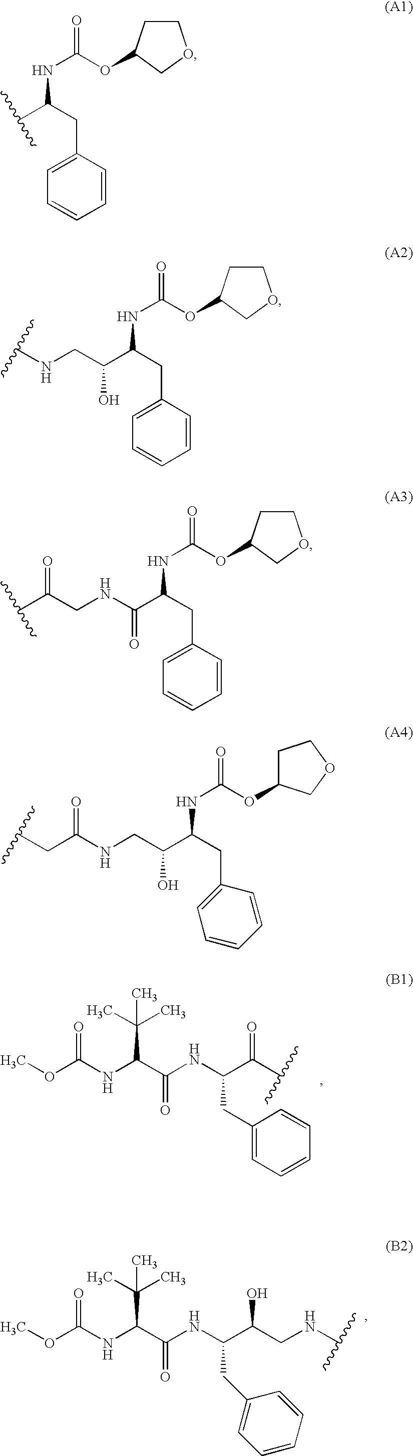

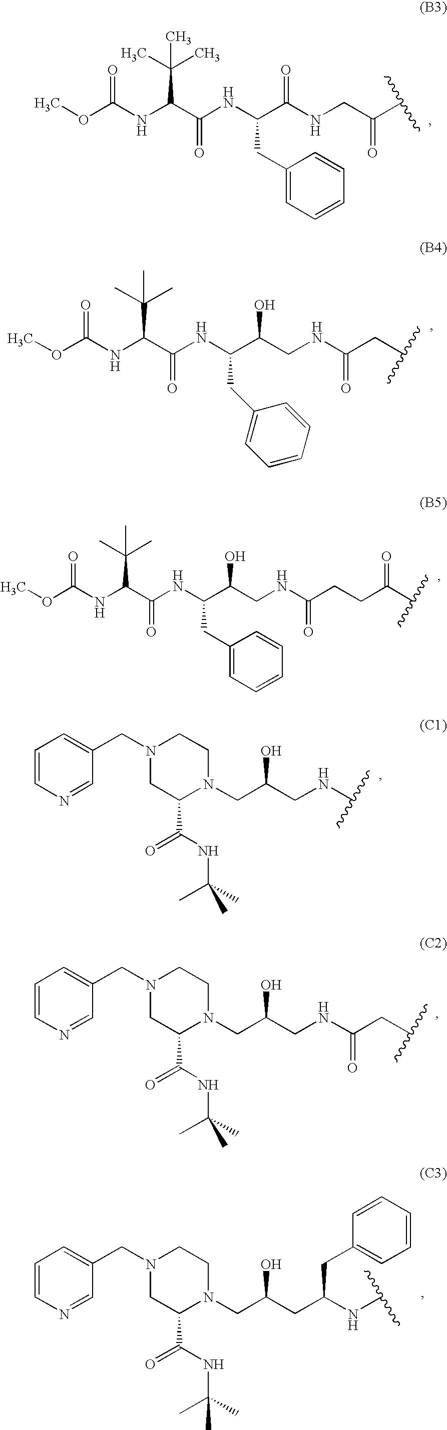

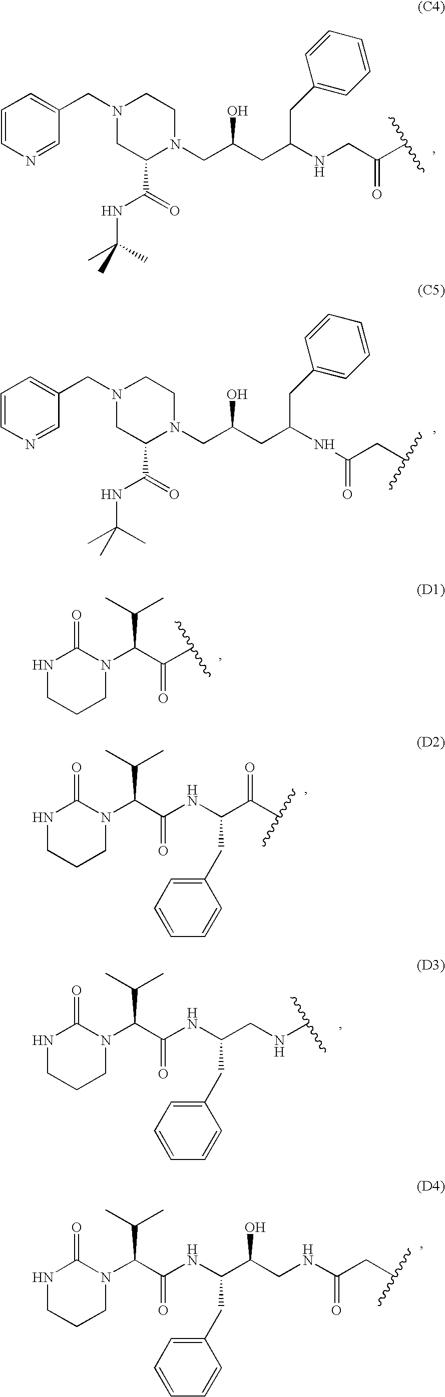

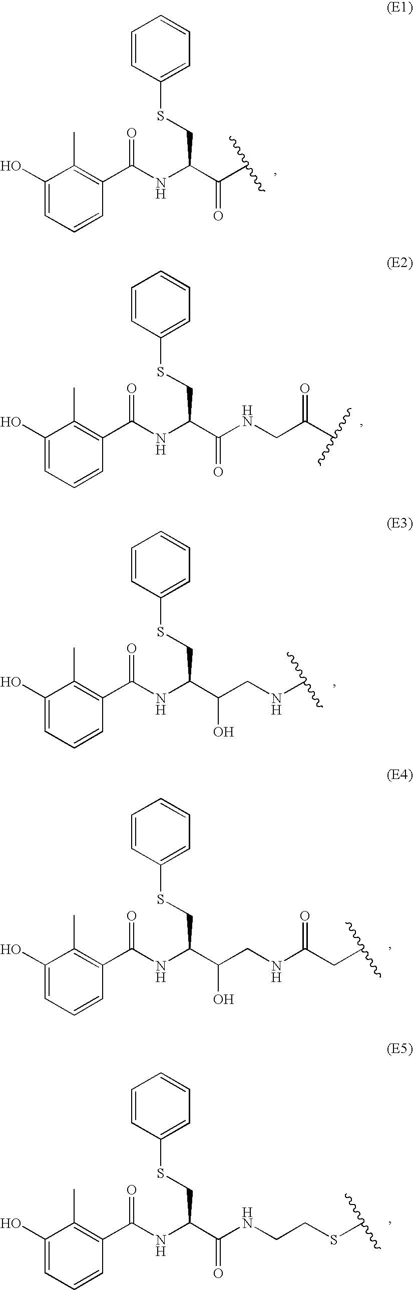

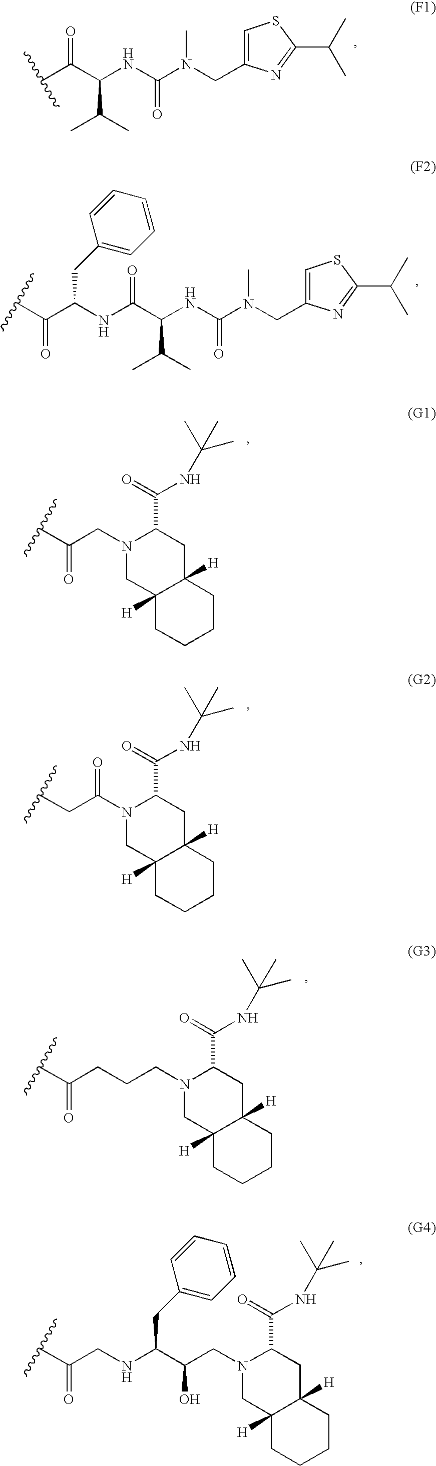

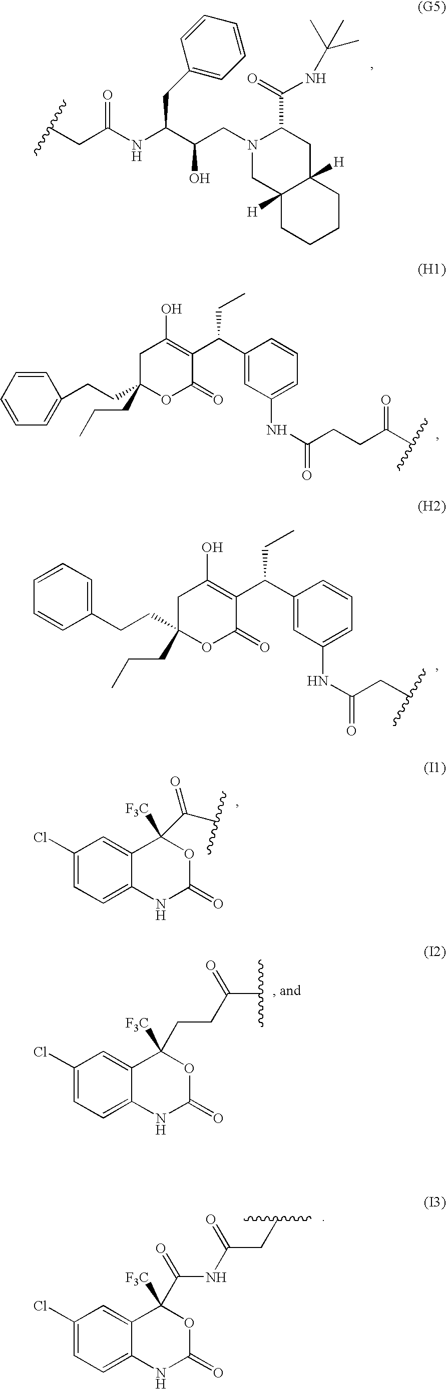

15. A compound having the structure:
[I—(X)ₖ—(C═O)ₘ—(Y)ₙ-(L)ₚ-Z]ᵣ—P
wherein I is a met-sensitive moiety of an anti-HIV therapeutic, wherein said anti-HIV therapeutic is selected from the group consisting of a protease inhibitor (PI) and a non-nucleoside HIV reverse transcriptase inhibitor (NNRTI);
X is selected from the group consisting of O, NH, and CH₂;
Y is selected from the group consisting of O, NH, CH₂, and CH₂—S;
k, m, n, and p are independently selected from 0 and 1;
L is a linker consisting of from 1 to 40 carbon atoms arranged in a straight chain or a branched chain, saturated or unsaturated, and containing up to two ring structures and 0-20 heteroatoms, with the provision that not more than two heteroatoms may be linked in sequence;
Z is a moiety selected from the group consisting of —CONH—, —NHCO—, —NHCONH—, —NHCSNH—, —OCONH—, —NHOCO—, —S—, —NH(C═NH)—, —N═N—, and —NH—;
P is a member selected from a polypeptide, a polysaccharide, a synthetic polymer, a carrier protein, an enzyme, a fluorogenic compound, and a chemiluminescent compound; and
r is a number from 1 to the number of hapten binding sites on P.

16. The compound of claim 15, wherein said I is a member selected from:

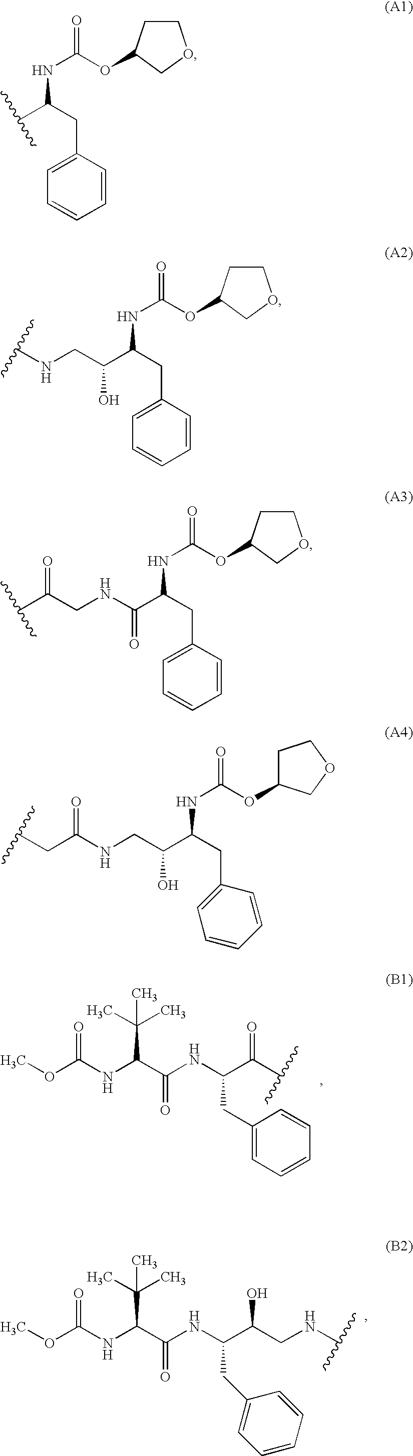

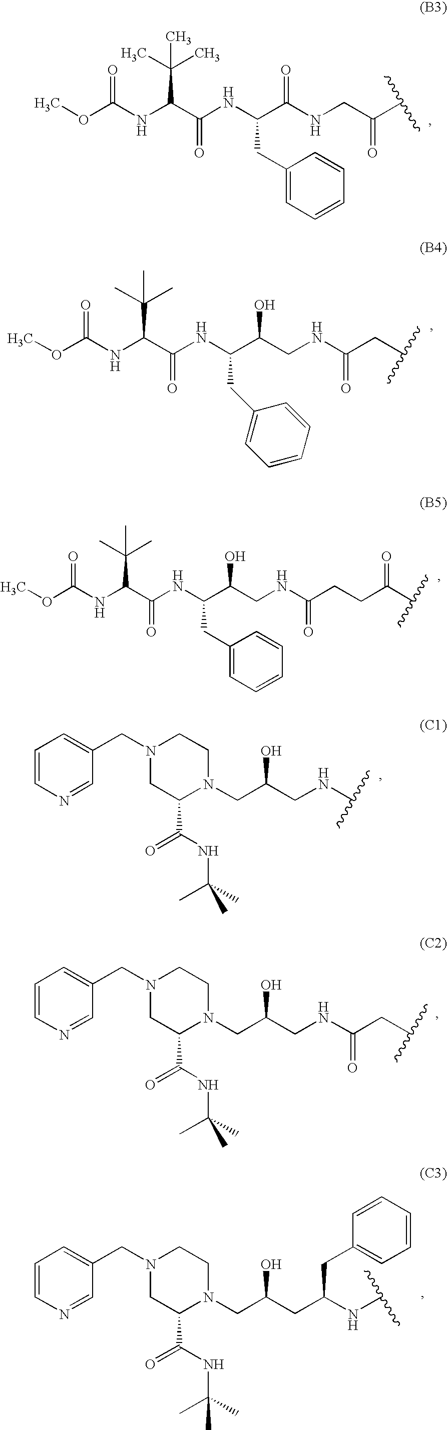

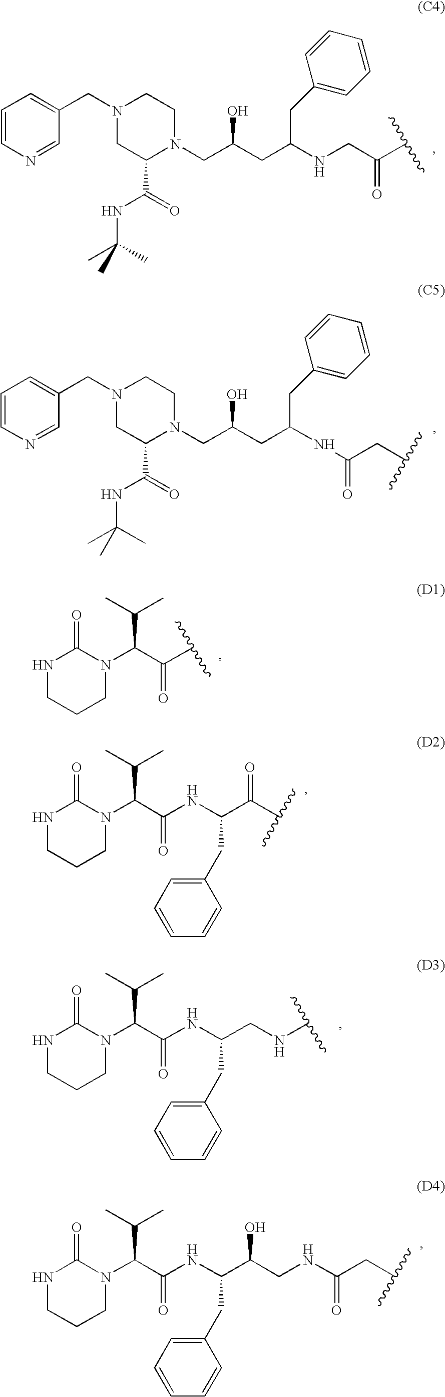

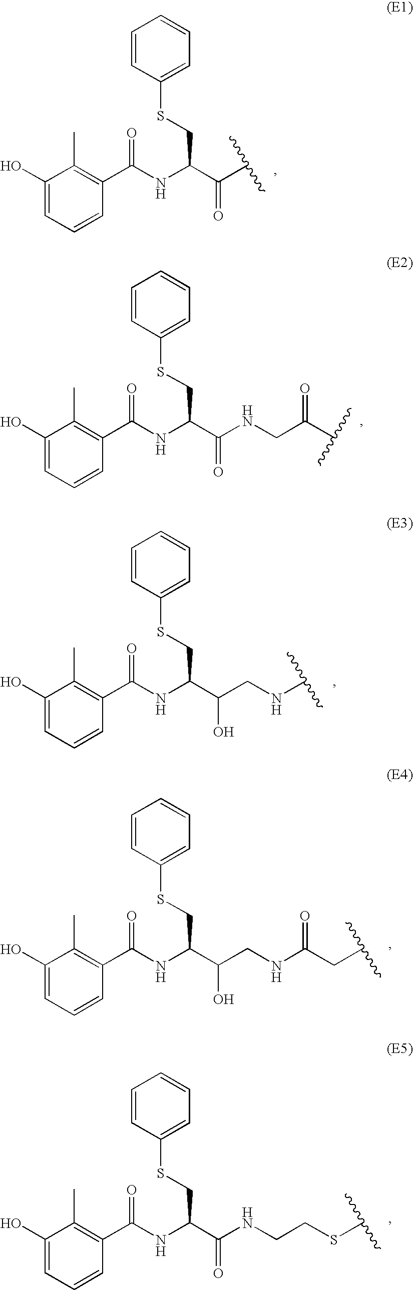

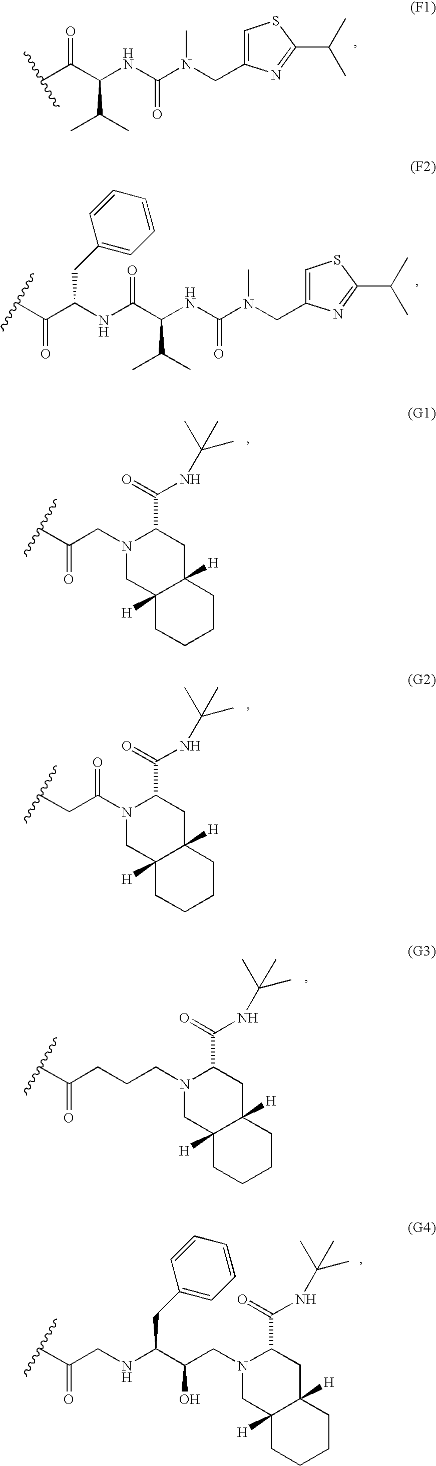

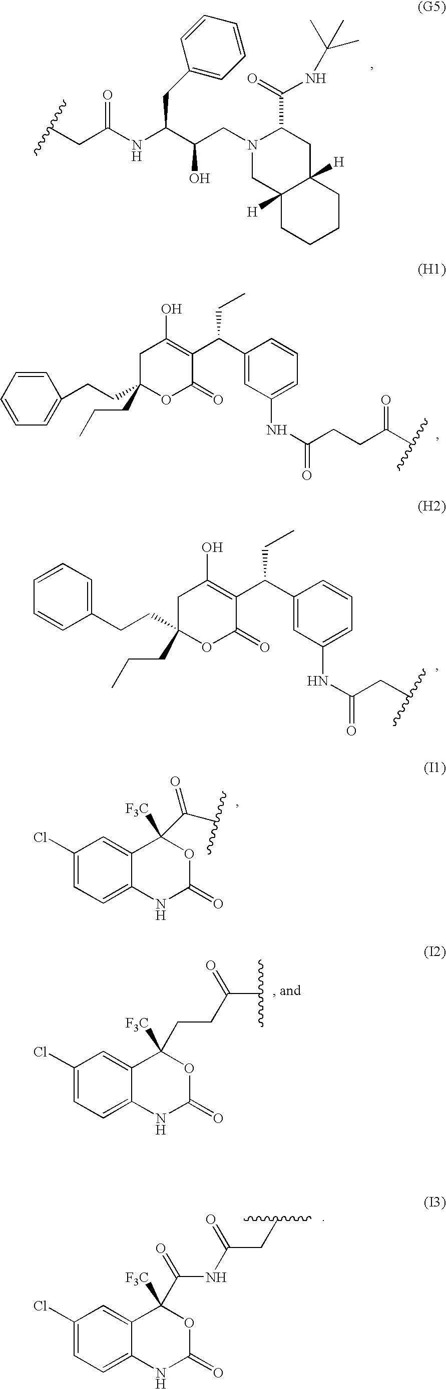

17. An antigen for generating an antibody specific for a met-sensitive moiety of an anti-HIV therapeutic.

18. A receptor that specifically binds to the compound of claim 10.

19. The receptor of claim 18, wherein said receptor is selected from a Fab, Fab′, F(ab′)′2, Fv fragment, and a single-chain antibody.

20. The receptor of claim 18, wherein said receptor is specific for a met-sensitive moiety of amprenavir and has less than 10% cross-reactivity with atazanavir, indinavir, lopinavir, nelfinavir, ritonavir, saquinavir, and tipranavir.

21. A receptor of claim 10, wherein I is a member selected from (A1), (A2), (A3), and (A4), and the receptor is a monoclonal antibody.

22. A receptor that substantially competes with the binding of the monoclonal antibody of claim 20 and the compound of claim 10, wherein I is a member selected from (A1), (A2), (A3), and (A4).

23. A receptor that substantially competes with the binding of the receptor of claim 21 and the compound of claim 10, wherein I is a member selected from (A1), (A2), (A3), and (A4).

24. The receptor of claim 23, wherein said receptor further comprises an antigen-binding domain.

25. A receptor that specifically binds to the compound of claim 15.

26. The receptor of claim 25, wherein said receptor is selected from a Fab, Fab′, F(ab′)2, Fv fragment, and a single-chain antibody.

27. The receptor of claim 25, wherein said receptor is specific for a met-sensitive moiety of amprenavir and has less than 10% cross-reactivity with atazanavir, indinavir, lopinavir, nelfinavir, ritonavir, saquinavir, and tipranavir.

28. A receptor of claim 15, wherein I is a member selected from (A1), (A2), (A3), and (A4), and the receptor is a monoclonal antibody.

29. A receptor that substantially competes with the binding of the monoclonal antibody of claim 27 and the compound of claim 15, wherein I is a member selected from (A1), (A2), (A3), and (A4).

30. A receptor that substantially competes with the binding of the receptor of claim 28 and the compound of claim 15, wherein I is a member selected from (A1), (A2), (A3), and (A4).

31. The receptor of claim 30, wherein said receptor further comprises an antigen-binding domain.

32. A method of generating antibodies, comprising administering a compound to a mammal, said compound having the structure:
[I—(X)ₖ—(C═O)ₘ—(Y)ₙ-(L)ₚ-Z]ᵣ—P
wherein I is a met-sensitive moiety of an anti-HIV therapeutic, wherein said anti-HIV therapeutic is selected from the group consisting of a protease inhibitor (PI) and a non-nucleoside HIV reverse transcriptase inhibitor (NNRTI);
X is selected from the group consisting of O, NH, and CH₂;
Y is selected from the group consisting of O, NH, CH₂, and CH₂—S;
k, m, n, and p are independently selected from 0 and 1;
L is a linker consisting of from 1 to 40 carbon atoms arranged in a straight chain or a branched chain, saturated or unsaturated, and containing up to two ring structures and 0-20 heteroatoms, with the provision that not more than two heteroatoms may be linked in sequence;
Z is a moiety selected from the group consisting of —CONH—, —NHCO—, —NHCONH—, —NHCSNH—, —OCONH—, —NHOCO—, —S—, —NH(C═NH)—, —N═N—, and —NH—;
P is a member selected from a polypeptide, a polysaccharide, a synthetic polymer, a carrier protein, an enzyme, a fluorogenic compound, and a chemiluminescent compound; and
r is a number from 1 to the number of hapten binding sites on P.

33. The method of claim 32, wherein said I is a member selected from:

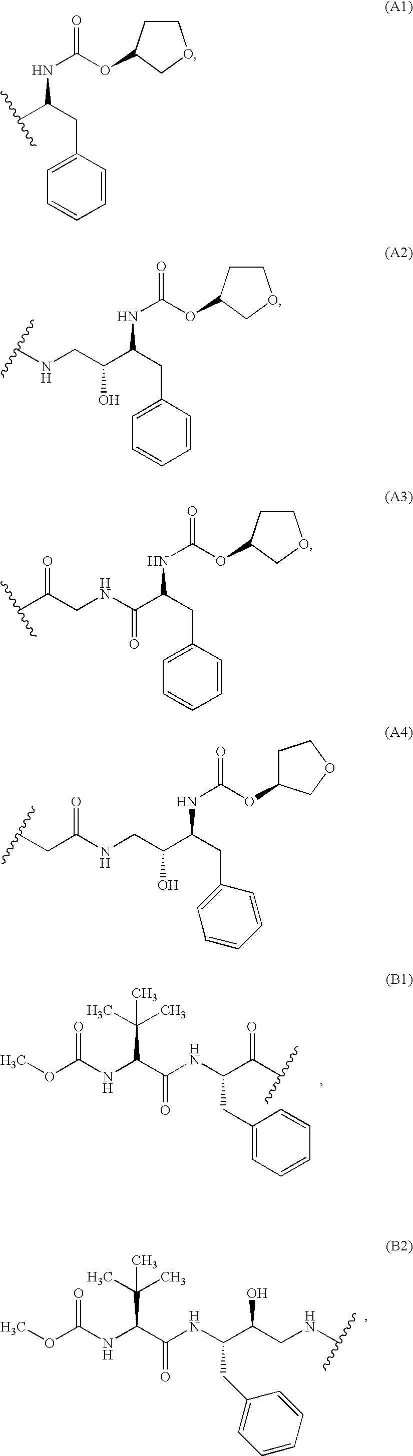

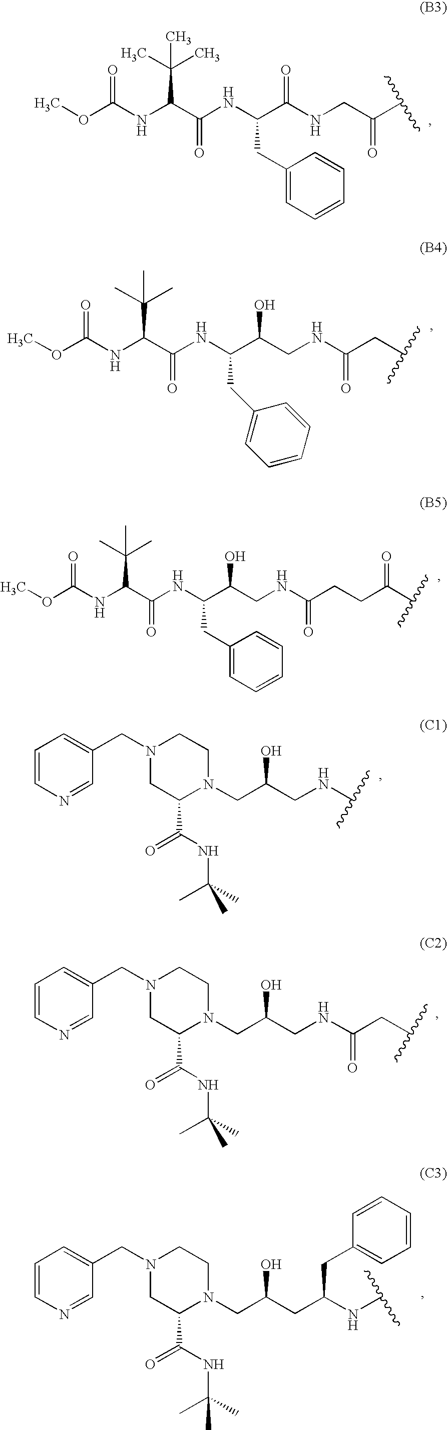

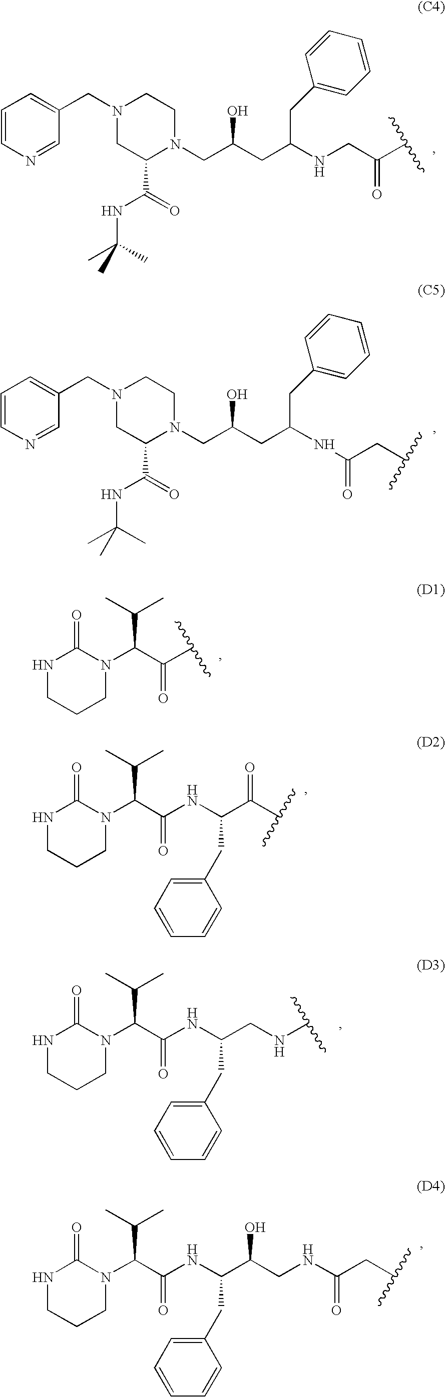

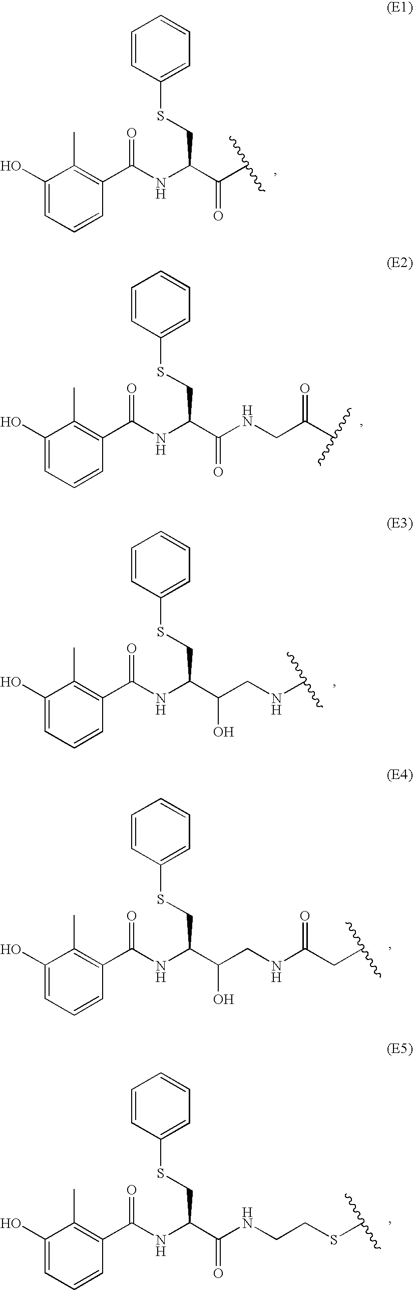

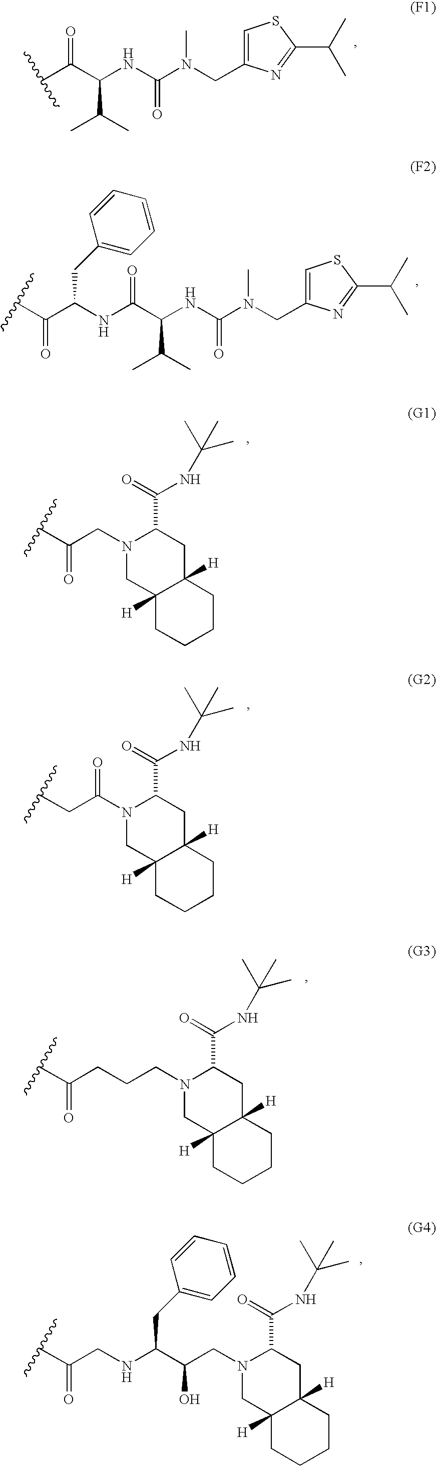

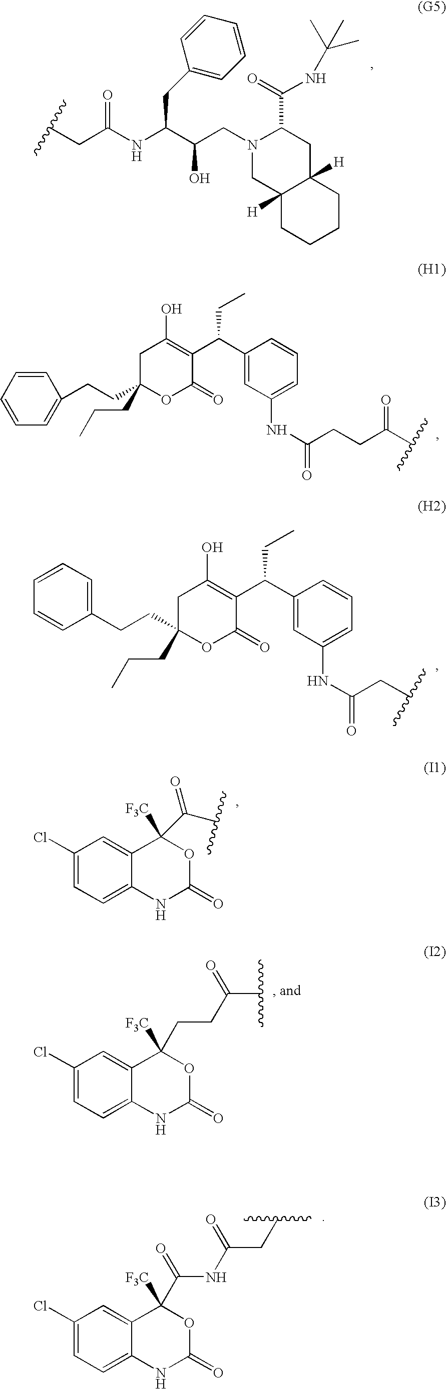

34. A kit for determining, in a sample from a host, the concentration of an anti-HIV therapeutic which inhibits HIV propagation, wherein said anti-HIV therapeutic is selected from the group consisting of a HIV protease inhibitor (PI) and a non-nucleoside HIV reverse transcriptase inhibitor (NNRTI) and said anti-HIV therapeutic comprises a met-sensitive moiety that is transformed by the host to yield an inactivated metabolic product,
said kit comprising:
(a) an antibody specific for said met-sensitive moiety where the antibody does not bind to said inactivated metabolic product, thus yielding an antibody-anti-HIV therapeutic complex; and
(b) a calibration standard.

35. The kit of claim 34, further comprising:
(c) instructions on the use of said kit; and
(d) a conjugate comprising said met-sensitive moiety and a non-isotopic signal generating moiety.

36. The kit of claim 34, wherein said signal generating moiety is selected from the group consisting of an enzyme, a fluorogenic compound, a chemiluminescent compound, and combinations thereof.

37. The kit of claim 34, wherein said PI is selected from the group consisting of amprenavir, atazanavir, indinavir, lopinavir, nelfinavir, ritonavir, saquinavir, and tipranavir.

38. The kit of claim 34, wherein said NNRTI is selected from the group consisting of efavirenz, nevirapine, delavirdine, and loviride.

39. The kit of claim 34, wherein said met-sensitive moiety is a member selected from:

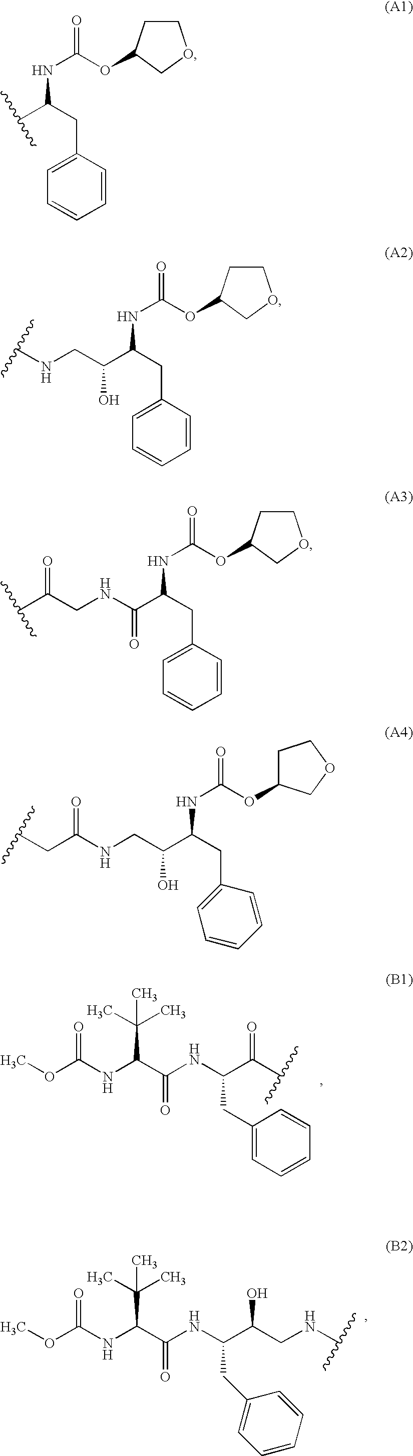

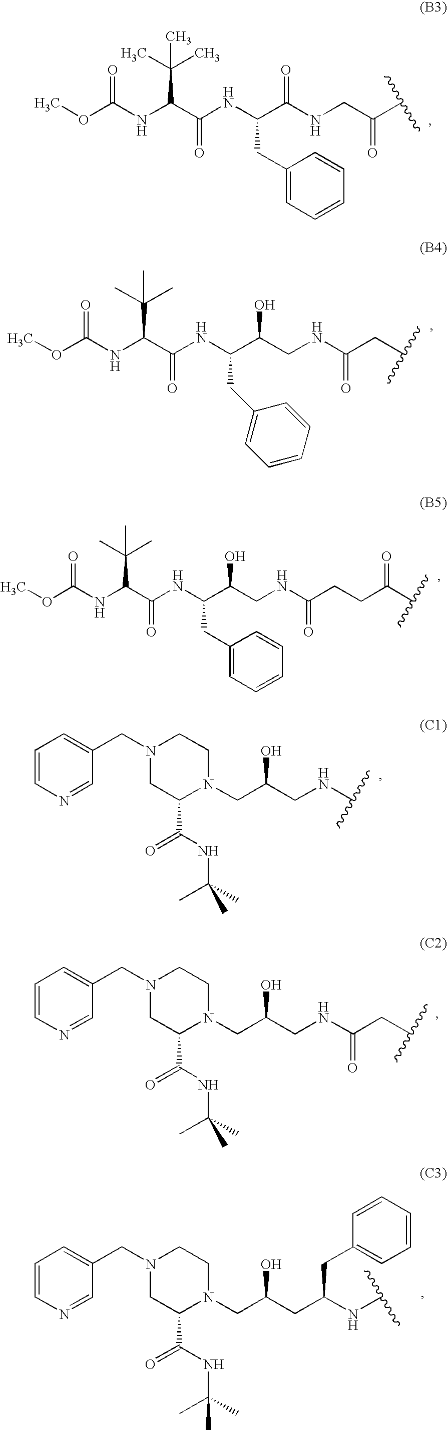

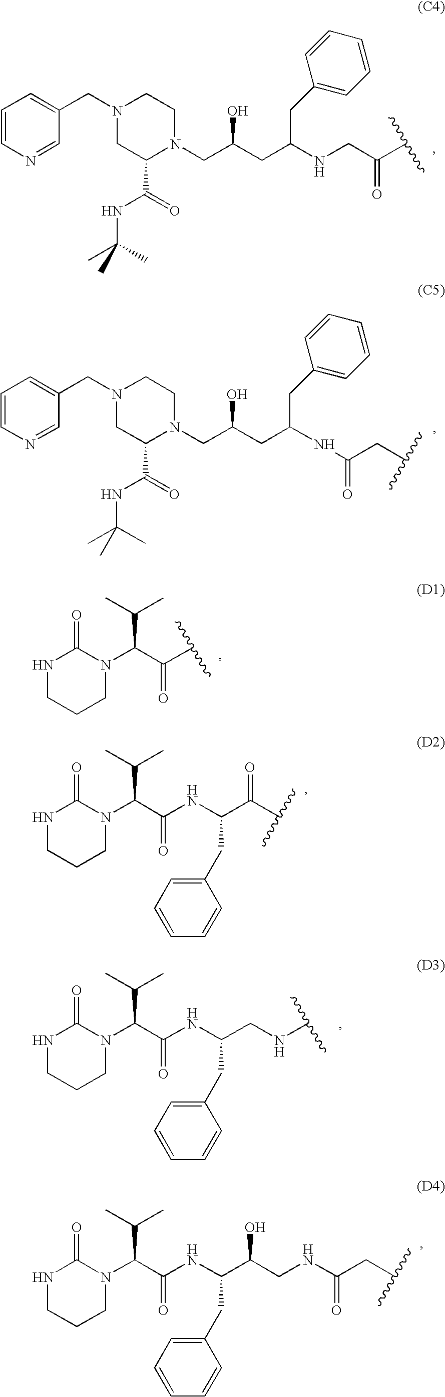

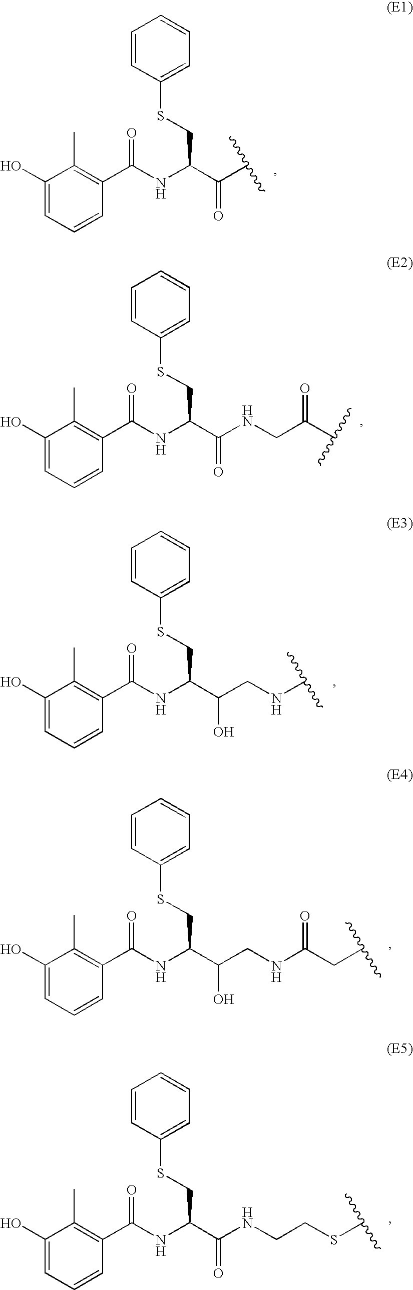

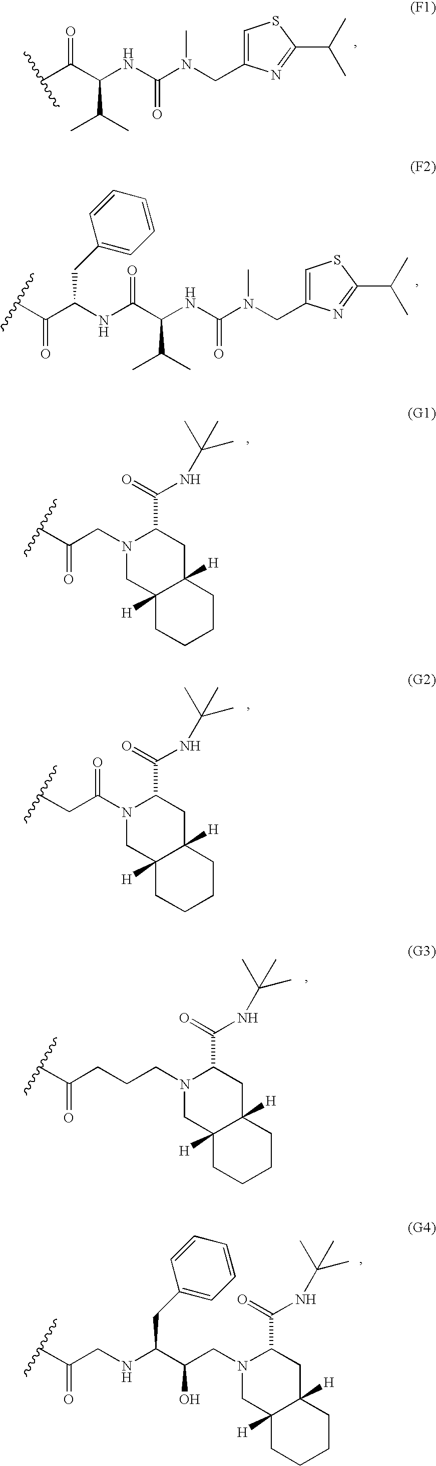

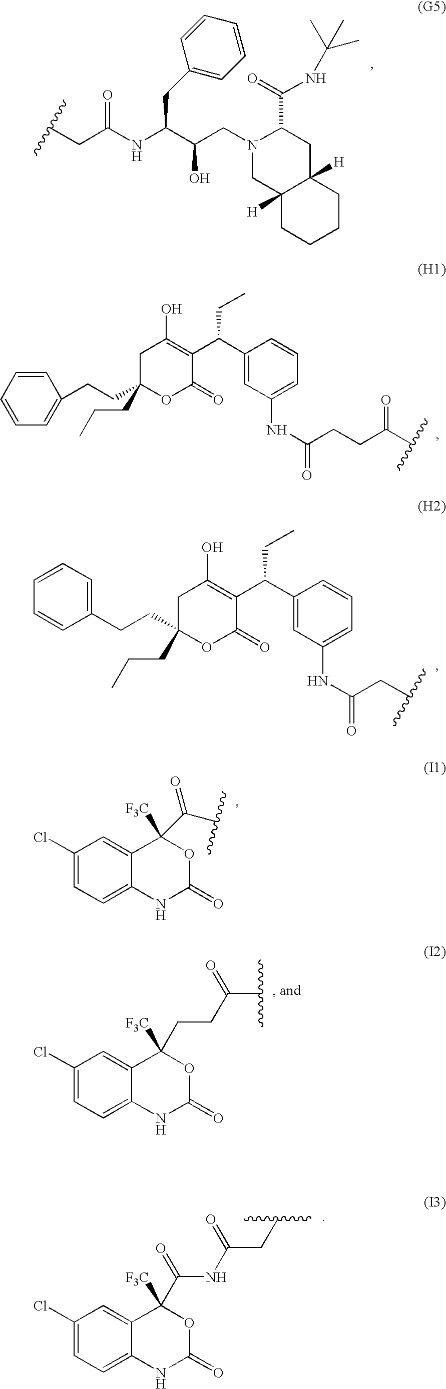

40. The kit of claim 34, wherein said calibration standard comprises a matrix selected from the group consisting of human serum and buffered synthetic matrix.

41. A method for determining, in a sample from a host, the presence or the concentration of a NNRTI, said method comprising:
(a) combining, in a solution, said sample with an antibody specific for said NNRTI, thus yielding an antibody-NNRTI complex; and
(b) detecting said complex.

42. The method of claim 41, wherein said antibody further comprises a non-isotopic signal-generating moiety.

43. The method of claim 41, wherein said NNRTI is selected from the group consisting of efavirenz, nevirapine, delavirdine, and loviride.

44. The method of claim 41, wherein said method is a homogeneous immunoassay.

45. The method of claim 41, wherein said detecting further comprises:
mixing said solution containing said antibody-NNRTI complex with a conjugate comprising said NNRTI and a non-isotopic signal generating moiety;
measuring the amount of said antibody bound to said conjugate by monitoring a signal generated by said non-isotopic signal generating moiety; and
correlating said signal with the presence or amount of said NNRTI in said sample.

46. The method of claim 42, wherein said signal generating moiety is selected from the group consisting of an enzyme, a fluorogenic compound, a chemiluminescent compound, and combinations thereof.

47. The method of claim 41, wherein said antibody is raised against an NNRTI derivative which is a member selected from:

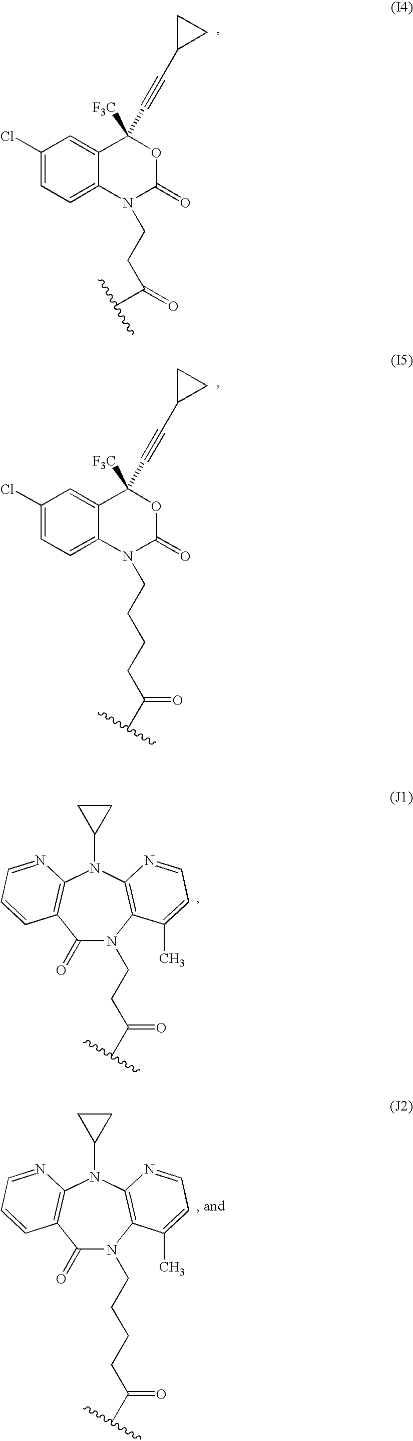

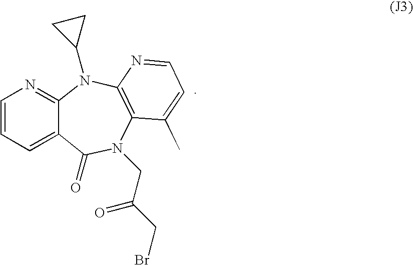

48. A compound having the structure:
I—(X)ₖ—(C═O)ₘ—(Y)ₙ-(L)ₚ-Q
wherein I is a NNRTI derivative;
X is selected from the group consisting of O, NH, and CH₂;
Y is selected from the group consisting of O, NH, CH₂, and CH₂—S;
k, m, n, and p are independently selected from 0 and 1;
L is a linker consisting of from 1 to 40 carbon atoms arranged in a straight chain or a branched chain, saturated or unsaturated, and containing up to two ring structures and 0-20 heteroatoms, with the provision that not more than two heteroatoms may be linked in sequence; and
Q is a reactive functional moiety chosen from the group consisting of active esters, halogens, isocyanates, isothiocyanates, thiols, imidoesters, anhydrides, maleimides, thiolactones, diazonium groups and aldehydes.

49. The compound of claim 48, wherein said I is a member selected from:

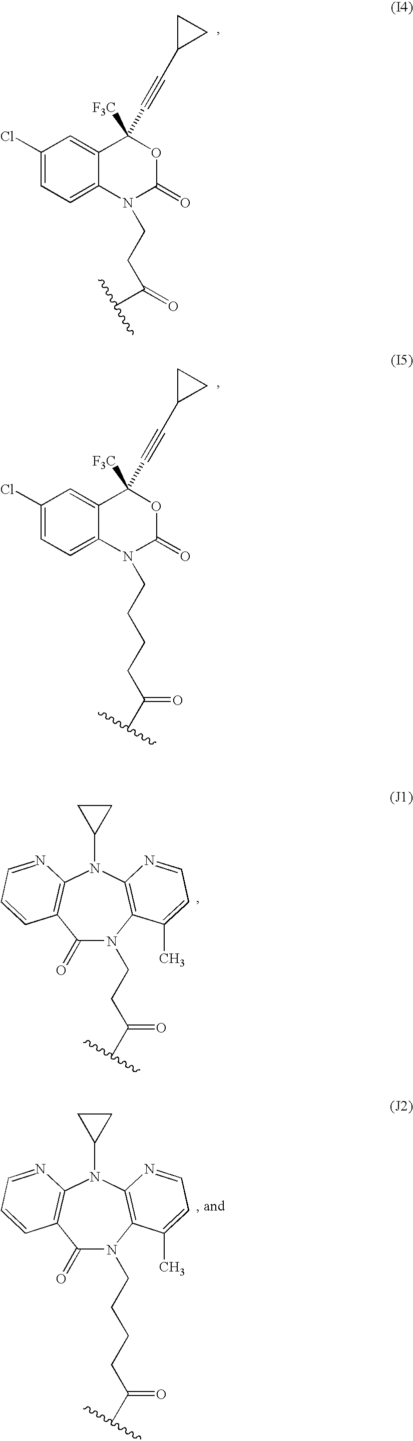

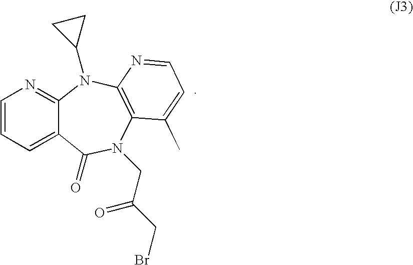

50. The compound of claim 48, wherein k is 1, X is O, m is 0, n is 0, p is 0, Q is succinimide, and I is a member selected from:

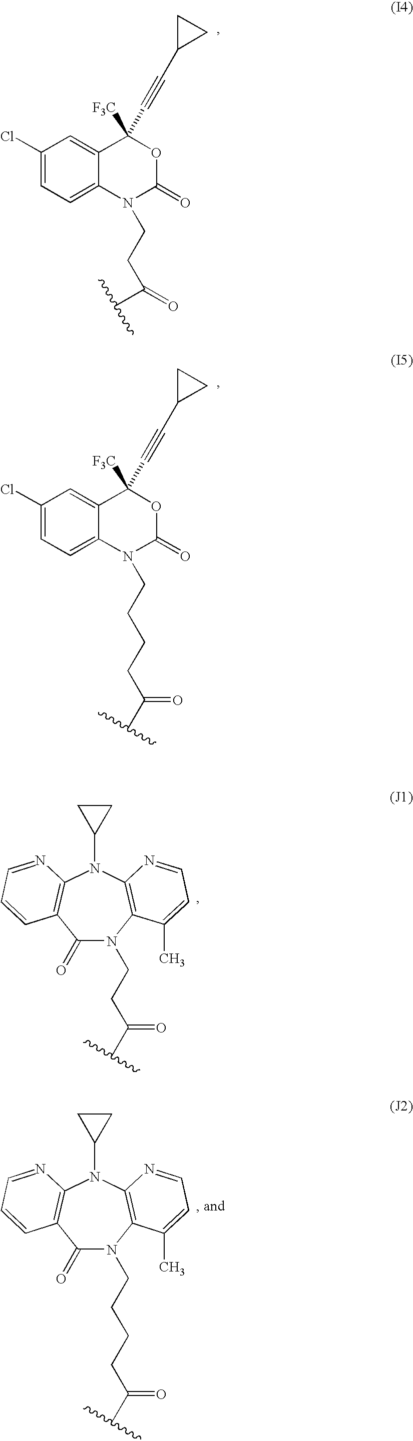

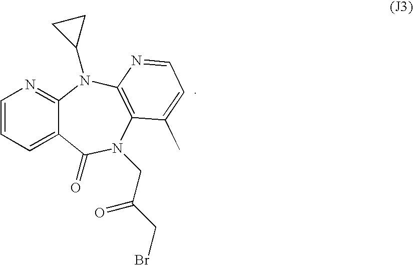

51. A compound having the structure:
[I—(X)ₖ—(C═O)ₘ—(Y)ₙ-(L)ₚ-Z]ᵣ-P
wherein I is a NNRTI derivative;
X is selected from the group consisting of O, NH, and CH₂;
Y is selected from the group consisting of O, NH, CH₂, and CH₂—S;
k, m, n, and p are independently selected from 0 and 1;
L is a linker consisting of from 1 to 40 carbon atoms arranged in a straight chain or a branched chain, saturated or unsaturated, and containing up to two ring structures and 0-20 heteroatoms, with the provision that not more than two heteroatoms may be linked in sequence;
Z is a moiety selected from the group consisting of —CONH—, —NHCO—, —NHCONH—, —NHCSNH—, —OCONH—, —NHOCO—, —S—, —NH(C═NH)—, —N═N—, and —NH—;
P is a member selected from a polypeptide, a polysaccharide, a synthetic polymer, a carrier protein, an enzyme, a fluorogenic compound, and a chemiluminescent compound; and
r is a number from 1 to the number of hapten binding sites on P.

52. The compound of claim 64, wherein said I is a member selected from:

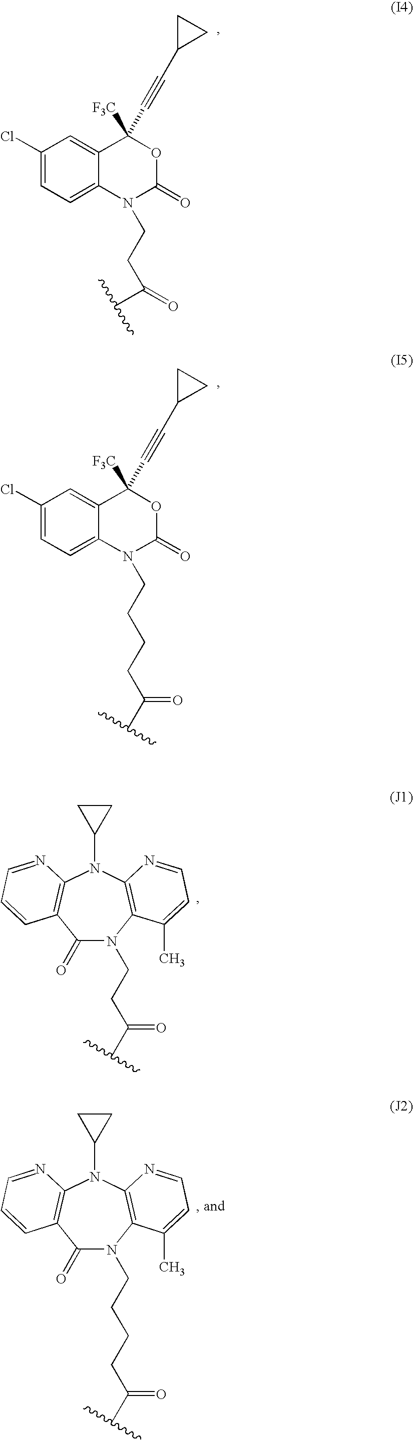

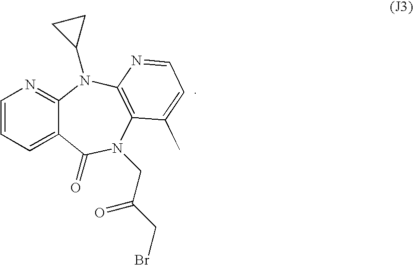

53. An antigen for generating an antibody specific for NNRTI.

54. A receptor that specifically binds to the compound of claim 48.

55. The receptor of claim 54, wherein said antibody or antigen-binding portion thereof is selected from a Fab, Fab′, F(ab′)2, Fv fragment, and a single-chain antibody.

56. The receptor of claim 54, wherein said receptor is specific for a met-sensitive moiety of amprenavir and has 10% or less cross-reactivity with atazanavir, indinavir, lopinavir, nelfinavir, ritonavir, saquinavir, and tipranavir.

57. A receptor of claim 54, wherein I is a member selected from (I4), (I5), (J1), (J2), and (J3), and the receptor is a monoclonal antibody.

58. A receptor that substantially competes with the binding of the receptor of claim 56 and the compound of claim 54, wherein I is a member selected from (I4), (I5), (J1), (J2), and (J3).

59. A receptor that substantially competes with the binding of the monoclonal antibody of claim 57 and the compound of claim 54, wherein I is a member selected from (I4), (I5), (J1), (J2), and (J3).

60. The receptor of claim 59, wherein said receptor further comprises an antigen-binding domain.

61. A receptor that specifically binds to the compound of claim 51.

62. The receptor of claim 61, wherein said receptor is selected from a Fab, Fab′, F(ab′)2, Fv fragment, and a single-chain antibody.

63. The receptor of claim 61, wherein said receptor is specific for a met-sensitive moiety of amprenavir and has less than 10% cross-reactivity with atazanavir, indinavir, lopinavir, nelfinavir, ritonavir, saquinavir, and tipranavir.

64. A receptor that specifically binds to the compound of claim 61, wherein I is a member selected from (I4), (I5), (J1), (J2), and (J3), and the receptor in a monoclonal antibody.

65. A receptor substantially competes with the binding of the receptor of claim 63 and the compound of claim 61, wherein I is a member selected from (I4), (I5), (J1), (J2), and (J3).

66. A receptor that substantially competes with the binding of the monoclonal antibody of claim 64 and the compound of claim 61, wherein I is a member selected from (I4), (I5), (J1), (J2), and (J3).

67. The receptor of claim 66, wherein said receptor further comprises an antigen-binding portion.

68. A method of generating antibodies, comprising administering a compound to a mammal, said compound having the structure:
[I—(X)ₖ—(C═O)ₘ—(Y)ₙ-(L)ₚ-Z]ᵣ-P
wherein I is a NNRTI derivative;
X is selected from the group consisting of O, NH, and CH₂;
Y is selected from the group consisting of O, NH, CH₂, and CH₂—S;
k, m, n, and p are independently selected from 0 and 1;
L is a linker consisting of from 1 to 40 carbon atoms arranged in a straight chain or a branched chain, saturated or unsaturated, and containing up to two ring structures and 0-20 heteroatoms, with the provision that not more than two heteroatoms may be linked in sequence;
Z is a moiety selected from the group consisting of —CONH—, —NHCO—, —NHCONH—, —NHCSNH—, —OCONH—, —NHOCO—, —S—, —NH(C═NH)—, —N═N—, and —NH—;
P is a member selected from a polypeptide, a polysaccharide, a synthetic polymer, a carrier protein, an enzyme, a fluorogenic compound, and a chemiluminescent compound; and
r is a number from 1 to the number of hapten binding sites on P.

69. The method of claim 68, wherein said I is a member selected from:

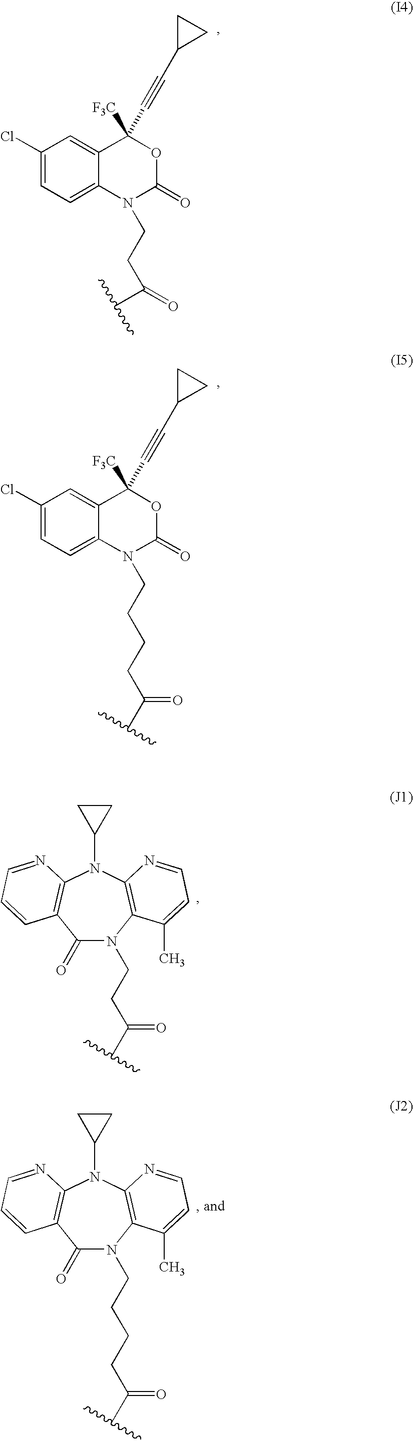

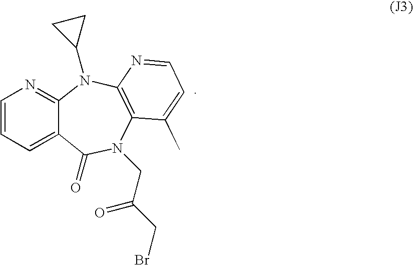

70. A kit for determining, in a sample from a host, the concentration of a NNRTI, said kit comprising:
(a) an antibody specific for said NNRTI, thus yielding an antibody-NNRTI complex; and
(b) a calibration standard.

71. The kit of claim 70, further comprising:
(c) instructions on the use of said kit;
(d). a conjugate comprising said NNRTI and a non-isotopic signal generating moiety.

72. The kit of claim 70, wherein said NNRTI Derivative is a member selected from:

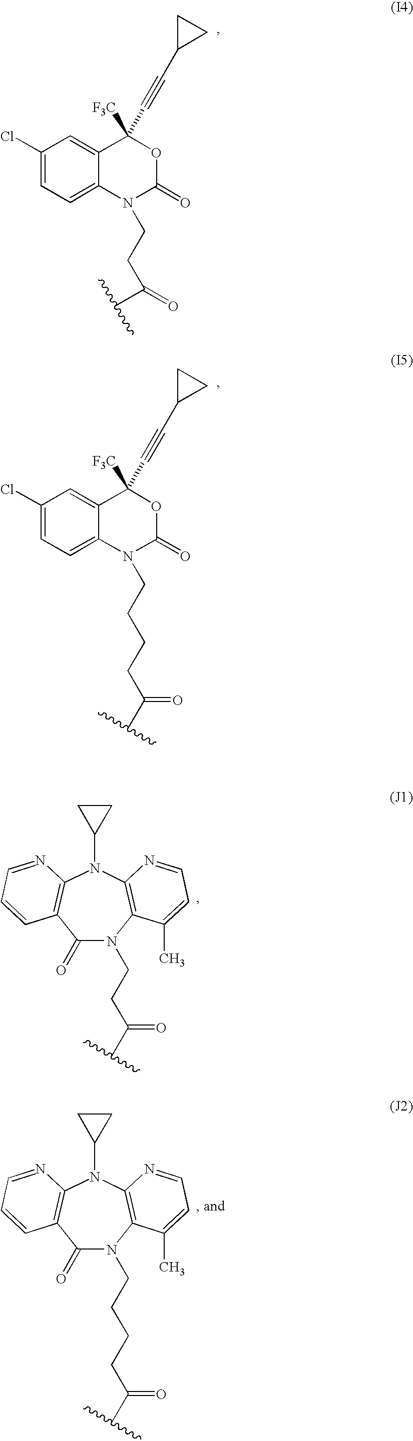

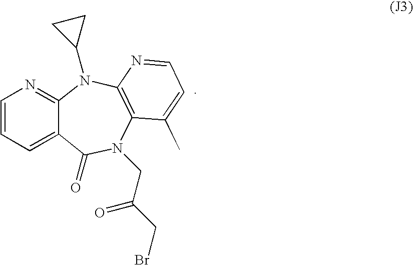

73. The kit of claim 70, wherein said calibration standard comprises a matrix selected from the group consisting of human serum and buffered synthetic matrix.
